(19) United States
(12) Patent Application Publication
(10) Pub. No.: US 2013/0209493 A1
(43) Pub. Date: Aug. 15, 2013

(54) BIOMAKERS FOR CIRCULATING TUMOR CELLS

(71) Applicants: Mariano Garcia-Blanco, Hillsborough, NC (US); Andrew Armstrong, Chapel Hill, NC (US); Daniel George, Chapel Hill, NC (US); Sebastian Oltean, Exeter (GB)

(72) Inventors: Mariano Garcia-Blanco, Hillsborough, NC (US); Andrew Armstrong, Chapel Hill, NC (US); Daniel George, Chapel Hill, NC (US); Sebastian Oltean, Exeter (GB)

(21) Appl. No.: 13/575,638

(22) PCT Filed: Sep. 24, 2010
(86) PCT No.: PCT/US10/50223

§ 371 Date: May 1, 2013

Related U.S. Application Data
(60) Provisional application No. 61/298,845, filed on Jan. 27, 2010.
(60) Provisional application No. 61/308,780, filed on Feb. 26, 2010.
(60) Provisional application No. 61/309,131, filed on Mar. 1, 2010.

(51) Int. Cl. G01N 33/68

(52) U.S. Cl. CPC G01N 33/6893
USPC 424/178.1; 435/7.1; 435/7.23

(57) ABSTRACT

Provided are methods for detecting circulating tumor cells (CTCs) in a subject. The methods may include detecting the expression of at least one epithelial mesenchymal transition (EMT) biomarker. Further provided are kits for detecting CTCs. The kits may include antibodies to at least one EMT biomarker. Further provided are methods of predicting the responsiveness of a subject to a cancer drug, methods of targeting delivery of a cancer drug in a subject, methods of providing a cancer prognosis to a subject, and methods for following the progress of cancer in a subject.

### CROSS-REFERENCE TO RELATED APPLICATIONS

This application claims the benefit of priority to U.S. Provisional Patent Application No. 61/298,845 filed Jan. 27, 2010; U.S. Provisional Patent Application No. 61/308,780 filed Feb. 26, 2010; and U.S. Provisional Patent Application No. 61/309,131 filed Mar. 1, 2010, which are all incorporated herein by reference in their entireties.

### STATEMENT REGARDING FEDERALLY SPONSORED RESEARCH

This invention was made with government support under federal grant number 5R33CA097502 from the NIH (NCI), and federal grant number 5K12CA10063904 from the NIH (NCI). The U.S. Government has certain rights to this invention.

### SEQUENCE LISTING

The sequence listing is filed with the application in electronic format only and is incorporated by reference herein. The sequence listing text file “B2442027.txt” was created on Sep. 24, 2010 and is 131,287 bytes in size.

### FIELD

The disclosure relates to methods for the detection and prognosis of cancer. Moreover, the disclosure provides methods for detecting circulating tumor cells (CTCs) that include the identification, detection, and optional enumeration of one or more biomarkers associated with CTCs that can be used in methods relating to a prognosis, diagnosis, or the treatment of cancer in a subject.

### BACKGROUND

Most metazoan cells can be classified as either epithelial or mesenchymal based on morphology, behavior and molecular signatures. Epithelial cells are generally polar in the apico-basal direction, adherent to adjacent cells in the plane perpendicular to the polarity, and non-motile in the polar direction. Mesenchymal cells, in contrast, lack polarity, do not form tight interactions with neighboring cells, and are motile. In adult animals epithelial and mesenchymal cells remain stably in one state or the other; that is, an epithelial cell does not change its properties and become mesenchymal. During development, however, epithelial cells of the early embryo give rise to all three embryonal layers (endoderm, mesoderm and ectoderm), which include mesenchymal cells (Hay, E. D., et al. Am. J. Kidney Dis. 1995, 26, 678-690). Therefore, these early embryonal cells have the ability to transition between epithelial and mesenchymal states, a property sometimes referred to as epithelial plasticity. Embryos have been shown to undergo epithelial-mesenchymal transitions (EMTs) as well as mesenchymal-epithelial transitions (METs) (Acloque, H., et al. J. Clin. Invest. 2009, 119, 1438-1449).

Circulating tumor cells (CTCs) are cells that have detached from a primary tumor and circulate in the bloodstream. CTCs may constitute seeds for subsequent growth of additional tumors (metastasis) in different tissues. Thus, detection of CTCs can provide for diagnosis and/or prognosis for overall survival and therapeutic implications in subjects with cancers such as metastatic prostate and breast cancer. The number of CTCs in any patient sample (e.g., a blood sample) can be very small, which can make detection difficult. Current methods for detecting CTCs are based on the detection of epithelial cell adhesion molecule (EpCAM) expression, which is a biomarker associated with epithelial cells. Such methods can under-detect CTCs under circumstances where cells undergo a decrease or loss of EpCAM expression, such as biologic processes including EMT. Because of the important role CTCs can play in the diagnosis, monitoring, and prognosis of disease in patients having cancer, any shortcoming in the detection technology needs to be addressed by the art.

Accordingly, there is a need for methods and systems for detecting CTCs that do not rely on existing capture technologies, and methods for correlating CTC detection to diagnosis, monitoring, and prognosis of disease in cancer patients.

### SUMMARY

In an aspect, the disclosure provides a method for detecting a circulating tumor cell (CTC) in a biological sample, the method comprising detecting at least one epithelial mesenchymal transition (EMT) biomarker in the biological sample.

In an aspect, the disclosure provides a kit for detecting a circulating tumor cell (CTC) in a biological sample, the kit comprising an antibody to at least one EMT biomarker and instructions for use.

In an aspect, the disclosure provides a method of predicting responsiveness of a subject having cancer to a course of cancer treatment, the method comprising: determining the level or presence of expression of at least one EMT biomarker to obtain an EMT biomarker profile and/or optionally a gene expression pattern for a CTC; and predicting the responsiveness of the subject to the cancer drug based on the EMT biomarker profile and/or optional gene expression pattern. In some embodiments the method includes: determining the level or presence of expression of at least one EMT biomarker in a sample from the subject to obtain a biomarker profile and optionally a gene expression pattern in a CTC for the subject; identifying the type of cancer from the biomarker profile and/or optional gene expression pattern, and optionally characterizing the stage of the cancer; and predicting responsiveness of the subject to the cancer drug based on any one of the biomarker pattern, the optional gene expression pattern, the type of cancer, or the stage of the cancer. Embodiments of this aspect can include detecting a number of cells captured and enumerated from a blood sample using at least one EMT biomarker applied to a sample from the subject. These cells that express the EMT biomarker are thereby captured using the EMT biomarker and could then be used to obtain a gene expression pattern in CTCs for the subject; to predict responsiveness of the subject to the cancer drug based on the obtained gene expression pattern, and for the detection of other biomarkers in these CTCs to assist in guiding therapy of that subject. These cells could also be used to measure the level of the specified EMT biomarker or other EMT biomarkers.

In an aspect, the disclosure provides a method of assessing the number of CTCs using both the traditional EpCAM based capture methodology and an EMT-marker based capture methodology. This EMT-based capture may replace or complement existing CTC capture technologies. The further capture, enumeration, and characterization of these CTCs using EMT antigen capture may further targeting delivery of a cancer drug in a subject having cancer comprising administering to the subject a cancer drug linked to an antibody specific for at least one EMT biomarker or specific drugs based on a gene expression profile or presence of this EMT biomarker.

In an aspect, the disclosure provides a method of estimating the prognosis of a subject with cancer as well as permitting a further characterization of CTCs that may predict for therapeutic responsiveness, the method comprising: determining the level of or presence of expression of at least one EMT biomarker in a sample from the subject to determine the number of CTCs in the subject and to obtain a gene expression pattern for the subject; and providing a prognosis to the subject based on the gene expression or biomarker profile pattern obtained.

In an aspect, the disclosure provides a method for monitoring progression of cancer in a subject undergoing therapeutic treatment, the method comprising detecting the level of expression or presence of expression of at least one EMT biomarker and the quantification of CTCs captured using this method in blood samples taken from the subject at a first and a second time; and comparing the first and second levels of expression; wherein a detected difference in the level of expression of the at least one EMT biomarker in the first and second samples over time indicates a change in the progression status of the cancer.

In an aspect, the disclosure provides a method for detecting cancer in a subject, the method comprising determining the presence of CTCs that express at least one EMT biomarker in a sample from the subject as compared to a normal or control sample, wherein an increased level of at least one EMT biomarker indicates presence of cancer progression or metastatic spread in the subject.

In an aspect, the disclosure provides a method of treating cancer in a subject comprising administering to the subject a cancer drug linked to an antibody that specifically binds at least one EMT biomarker.

Other aspects and embodiments of the disclosure will become apparent by consideration of the detailed description and accompanying drawings.

### BRIEF DESCRIPTION OF THE DRAWINGS

FIG. 1. (A) depicts a schematic representation of the IIIb and IIIc alternatively spliced isoforms of FGFR2. (B) is a schematic of the pRIIIc1² minigene and the fluorescence read-out. (C) is an RT-PCR analysis of the reporter (upper panel) and endogenous FGFR2 (lower panel). (D) are epifluorescence and phase-contrast pictures of clones AT3-M and AT3-T.

FIG. 2. (A) depicts examples of clusters of DsRED positive cells formed by AT3-M cells upon treatment with conditioned media from clone AT3-T. (B) depicts flow cytometry analysis of the same experiment.

FIG. 3. (A) depicts growth curves for clones AT3-T and AT3-M. (B) is graph of growth of AT3-M, AT3-T, and DT cells in soft agar. (C) depicts a sacrifice curve for rats injected with AT3-M or AT3-T cells. (D) depicts a comparison of tumor volumes resulting from AT3-T and AT3-M injection.

FIG. 4. (A) a representative example of cells that express both RFP and GFP at the periphery of an AT3-M tumor stably transfected with Gint and pRIIIc1² reporters. (B) a representative example of a section from an AT3-T tumor stably transfected with GFP and pRIIIc1² reporters.

FIG. 5 a representative example of cells that express both RFP and GFP at the periphery of an AT3-M tumor stably transfected with Gint and pRIIIc1² reporters.

FIG. 6. (A) representative pictures of cells for the scratch-wound assay. (B) a quantification of migration. (C) an invasion assay using Matrigel coated membranes. (D) a quantification of invasion assay results.

FIG. 7 are metastatic foci in lungs from animals with tumors from either AT3-T or AT3-M clones (stably transfected with GFP and pRIIIc1² reporters). (A) (upper panel) is an example of a section exhibiting the pattern for clone AT3,-T (i.e. GFP+, DsRED+) in a metastatic focus and (lower panel) an example of a section exhibiting a plastic pattern for clone AT3-T (i.e. GFP+, DsRED−) in a metastatic focus. (B) (upper panel) is an example of a section exhibiting the pattern for clone AT3-M (i.e. GFP+, DsRED−) in a metastatic focus and (lower panel) an example of a section exhibiting a plastic pattern for clone AT3-M (i.e. GFP+, DsRED+) in a metastatic focus.

FIG. 8A a membrane with serial two-fold dilutions of whole cell lysates cut in half and immunoblotted for CD133 (upper panel) or β-actin (lower panel). (B) a membrane with serial twofold dilutions of whole cell lysates cut in half and immunoblotted for CD44 (upper panel) or β-actin (lower panel).

FIG. 9 depicts a model comparing stem cell-like character and epithelial mesenchymal phenotype.

FIG. 10 depicts CTCs from patients with prostate adenocarcinoma. (A) illustrates an example of a leukocyte from a human peripheral blood mononuclear cell (PMBC) sample: CD45 (+), CK (−), and vimentin (+). (B) illustrates an example of a CD45 (−), CK (+), and vimentin (−) cell from a patient with metastatic breast cancer. (C) illustrates an example of a CD45 (−), CK (+), vimentin (+) from a patient with metastatic breast cancer (mBC). (D) illustrates an example of a CD45 (−), CK (+), vimentin (+) from a patient with metastatic progressive castrate-resistant prostate cancer (mCRPC).

FIG. 11 depicts immunofluorescent images of CTCs from patients with mCRPC and mBC.

FIG. 12 depicts immunofluorescent images of CTCs from patients with mCRPC and mBC.

FIG. 13 depicts immunofluorescent images of CTCs from patients with mCRPC and mBC.

FIG. 14 depicts immunofluorescent images of CTCs from patients with mCRPC and mBC.

FIG. 15 depicts immunofluorescent images of CTCs from patients with mCRPC and mBC.

FIG. 16 depicts immunofluorescent images of CTCs from patients with mCRPC and mBC.

### DETAILED DESCRIPTION

Before any embodiments are described in detail, it is to be understood that the claims are not limited to the details of construction and the arrangement of components set forth in the following description or illustrated in the included drawings.

In a general sense, the disclosure provides biomarkers that have been identified to be associated with circulating tumor cells (CTCs). As described herein, one or more biomarkers of epithelial mesenchymal transition (EMT) are detectable on CTCs of patients afflicted with common epithelial malignancies. These transitional cells often display stem cell-like characteristics (sternness) and/or plasticity. Further, the disclosure provides description that metastatic propensity and epithelial phenotypic changes correlate with alternative splicing of the FGFR2 gene. The disclosure also provides that, as illustrated in the non-limiting Examples, transitional cells are found in cancer patients where many CTCs co-expressed biomarkers associated with epithelial and mesenchymal cells.

Thus, as described below EMT biomarker expression can be used to detect and quantify CTCs in a biological sample. Accordingly, methods comprising detection of EMT biomarker expression, or detection of CTCs, or a combination thereof, can be used to assess cancer prognosis, tumor invasiveness, risk of metastasis, or to stage tumors. As one of skill in the art will appreciate, any suitable method for evaluating EMT biomarker expression can be used to evaluate EMT biomarker expression according to the methods described herein including, but not limited to, detection with antibodies, real time RT-PCR, Northern analysis, Western analysis, and flow cytometry.

As described herein the ability for a cell to transition easily between epithelial-like and mesenchymal-like states (phenotypic plasticity) is a relevant determinant of malignant fitness more so than the properties of the end states. While these epithelial transitions are phenotypic, the propensity to transition (plasticity) among carcinoma cells may be determined by genotype. The majority of plastic cells may inhabit transitional intermediate states with properties of both epithelium and mesenchyme, and that these transitional cells may be particularly malignant. Such cells may be detected in: (1) tumors where the cancer cells have mixed histology, which indeed have been observed and have been classified as highly aggressive (e.g., clonal sarcomatous carcinomas of epithelial origin, which exhibit an extremely aggressive behavior, such as sarcomatoid renal cell carcinoma and carcinosarcoma of the prostate); and (2) cancer cells co-expressing epithelial and mesenchymal markers, as described herein.

The disclosure, as illustrated by the non-limiting embodiments in the Examples, provides for identification of cells that possess an intermediate phenotype—expressing epithelial and mesenchymal isoforms of FGFR2, having epithelial-like morphology and gene expression patterns, while also displaying mesenchymal cell-like migration, tumor formation, and metastases. In embodiments, these cells are identified in patients with advanced cancer, metastatic adenocarcinoma, and metastatic breast and prostate carcinomas. In some embodiments, the cells comprise CTCs. In some embodiments the CTCs co-expresses biomarkers including, for example, EpCAM, cytokeratin, and vimentin, which identify cells as both epithelial- and mesenchymal-like. In some embodiments, these CTCs in intermediate phenotypic states are identified by detecting EMT biomarkers and provide a diagnosis and/or prognosis of the state and/or degree of malignancy of a cancer.

In an aspect the disclosure provides a method for detecting CTCs in a biological sample, the method comprising detecting at least one epithelial mesenchymal transition (EMT) biomarker in the biological sample. In some embodiments such as illustrated in the Examples, biomarkers of EMT are present on the CTCs of patients with common epithelial malignancies. In some embodiments methods that include detection and identification of alternative splice variants of the FGFR2 gene are used to correlate to metastatic propensity and epithelial phenotypic in a CTC.

Thus, EMT biomarker expression may be used to detect CTCs. EMT biomarker expression, or detection of CTCs, or a combination thereof, may be used to assess cancer prognosis, tumor invasiveness, risk of metastasis, or to stage tumors. As mentioned above, the methods described herein can include any suitable method for evaluating EMT biomarker expression including, but not limited to, detection with antibodies, real time RT-PCR, Northern analysis, magnetic particles (e.g., microparticles or nanoparticles), Western analysis, and any method or system involving flow cytometry. In some embodiments, the methods and EMT biomarkers can be used in a commercially available system such as a system that has been approved by a regulatory agency (e.g., FDA) including, for example, CellSearch® technology (Veridex LLC). Thus, the methods can incorporate standard protocols that are known in the art. For example, embodiments comprising CellSearch® technology can include detecting the presence of an EMT biomarker, and correlated to quantifying the number of circulating tumor cells (CTCs) a biological sample, (e.g., blood collected from women in need of a new treatment regimen for metastatic breast cancer, or men in need of treatment for mCRPC). Typical protocols can include drawing blood sample sizes of about 15 mL that can be collected at any particular time (suitably when the patient starts the new therapy, and then again at three to four week intervals). The number of CTCs can be correlated with disease response or progression as determined by standard radiology studies (e.g., CT scans) performed every nine to 12 weeks.

In an aspect, the disclosure relates to a method for detecting a circulating tumor cell (CTC) in a biological sample, wherein the method comprises detecting at least one epithelial mesenchymal transition (EMT) biomarker in the biological sample. As noted above, a biological sample can be from any tissue or fluid from an organism. In some embodiments the biological sample is from a bodily fluid or tissue that is part of, or associated with, the lymphatic system or the circulatory system of the organism. In some embodiments the biological sample is a blood sample.

The epithelial mesenchymal transition (EMT) and cellular plasticity biomarkers used in the methods described herein are associated with circulating tumor cells (CTCs). Accordingly, in various embodiments the methods include detecting the presence of one or more EMT biomarker and correlating that detection with the presence of a CTC, optionally quantifying the number of CTCs in the sample. As discussed herein, EMT biomarkers can include any detectable biomolecule that is associated with a transitional cell that exhibits characteristics (e.g., phenotype, or surface antigen or gene expression profiles, etc.) of plasticity, stem-like properties, invasiveness, and/or chemo-resistance of a cell. In some non-limiting embodiments, the EMT biomarker includes any of vimentin, N-cadherin, O-cadherin, E-cadherin, FGFR2 splice variant isoforms (such as, for example FGFR2 that includes or excludes either exon IIIc or exon IIIb), or CD133, or any combination of two or more thereof. In some embodiments, the EMT biomarker can include one or more of vimentin (polypeptide SEQ ID NO: 14 encoded by polynucleotide SEQ ID NO: 13), N-cadherin (polypeptide SEQ ID NO: 2 encoded by polynucleotide SEQ ID NO: 1; polypeptide SEQ ID NO: 16 encoded by polynucleotide SEQ ID NO: 15), O-cadherin (polypeptide SEQ ID NO: 4 encoded by polynucleotide SEQ ID NO: 3; polypeptide SEQ ID NO: 18 encoded by polynucleotide SEQ ID NO: 17), E-cadherin (polypeptide SEQ ID NO: 12 encoded by polynucleotide SEQ ID NO: 11; polypeptide SEQ ID NO: 24 encoded by polynucleotide SEQ ID NO: 23), FGFR2 (polypeptide SEQ ID NO: 8 encoded by polynucleotide SEQ ID NO: 7; polypeptide SEQ ID NO: 10 encoded by polynucleotide SEQ ID NO: 9; polypeptide SEQ ID NO: 22 encoded by polynucleotide SEQ ID NO: 21), and CD133 (polypeptide SEQ ID NO: 6 encoded by polynucleotide SEQ ID NO: 5; polypeptide SEQ ID NO: 20 encoded by polynucleotide SEQ ID NO: 19). In some embodiments, the EMT biomarker can include one or more of N-cadherin, for example human N-cadherin (for example SEQ ID NO: 16, CCDS ID No: CCDS11891.1); O-cadherin, for example human O-cadherin (for example SEQ ID NO: 18, CCDS ID No: CCDS10803.0); E-cadherin, for example human E-cadherin (for example SEQ ID NO: 24, CCDS ID No: CCDS10869.1); CD133, for example human CD133 (for example SEQ ID NO: 20, CCDS ID No: CCDS47029.1); FGFR2, for example human FGFR2 (for example SEQ ID NO: 22, CCDS ID No: CCDS31298.1); and vimentin, for example human vimentin (for example SEQ ID NO: 14, Accession No. BC000163). It will be understood by one of skill in the art that when reference is made to polynucleotides that encode polypeptides in the above embodiments as well as embodiments throughout, the polynucleotide can be disclosed as either an RNA (e.g., mRNA) or a DNA (e.g., cDNA).

The EMT biomarkers can be associated with any organism (ortholog) and in certain embodiments are EMT biomarkers associated with a human. Any portion or the entirety of an EMT biomarker can be used for detecting in the methods described herein such as, for example, an epitope of an EMT biomarker protein that binds to an antibody, or a nucleic acid sequence of an EMT biomarker an expressed or transcribed mRNA molecule that is complementary to a reporter nucleic acid probe or primer. In some embodiments, the methods provide for detecting expression of at least two EMT biomarkers. In certain embodiments, expression of vimentin and E-cadherin are detected. In certain embodiments, expression of N-cadherin and O-cadherin are detected. This measure may be used alone or in combination with another method to detect CTCs. In certain embodiments, the methods described herein may be used as a supplemental method in conjunction with CellSearch® Circulating Tumor Cell Test (noted above). Thus, embodiments provide for a method as part of a dual or complementary detection system that can be used to detect and optionally quantify CTCs in a sample (e.g., comprising the detection of EpCAM and at least one EMT biomarker). The expression of at least one EMT biomarker may be used to isolate CTCs. The expression of at least one EMT biomarker may be used to count or provide a relative number or amount of CTCs, using any known method for correlating detection of a biomarker to a cell, such as a CTC. CTCs may be detected at the time of, prior to, or after metastasis.

Cancers may include, but are not limited to, breast cancer, colon cancer, lung cancer, prostate cancer, testicular cancer, brain cancer, skin cancer, rectal cancer, gastric cancer, esophageal cancer, sarcomas, tracheal cancer, head and neck cancer, pancreatic cancer, liver cancer, ovarian cancer, lymphoid cancer, cervical cancer, vulvar cancer, melanoma, mesothelioma, renal cancer, bladder cancer, thyroid cancer, bone cancers, carcinomas, sarcomas, and soft tissue cancers. Thus, the disclosure is generally applicable to any type of cancer in which expression of an EMT biomarker occurs. In certain embodiments, the cancer is a solid tumor malignancy. In certain embodiments, the cancer is breast, colon, or prostate cancer.

Expression of at least one EMT biomarker may be detected using any suitable method known in the art, including but not limited to, binding with antibodies or fragment thereof, antibodies tethered to or associated with an imaging agent, expression reporter plasmids, flow cytometry, and any suitable array scanner technology. The antibody or fragment thereof may suitably recognize a particular intracellular protein, protein isoform, or protein configuration.

As used herein, an “imaging agent” or “reporter molecule” is any entity which enhances visualization or detection of the cell to which it is delivered. Any type of detectable reporter molecule/imaging agent can be used in the methods disclosed herein for the detection of one or more EMT biomarker. Such detectable molecules are known in the art and include, for example, magnetic beads, fluorophores, radionuclides, nuclear stains (e.g., DAPI). For example, an imaging agent can include a compound that comprises an unstable isotope (i.e., a radionuclide) or a fluorescent moiety, such as Cy-5, Alexa 647, Alexa 555, Alexa 488, fluorescein, rhodamine, and the like. Suitable radionuclides include both alpha- and beta-emitters. In some embodiments, the targeting vehicle is labeled. In other embodiments, suitable radioactive moieties include labeled polynucleotides and polypeptides which can be coupled to the targeting vehicle. In some embodiments, the imaging agent comprises a radionuclide such as, for example, a radionuclide that emits low-energy electrons (e.g., those that emit photons with energies as low as 20 keV). Such nuclides can irradiate the cell to which they are delivered without irradiating surrounding cells or tissues. Non-limiting examples of radionuclides that are can be delivered to cells include ¹³⁷Cs, ¹⁰³Pd, ¹¹¹In, ¹²⁵I, ²¹¹At, ²¹²Bi and ²¹³Bi, among others known in the art. Further imaging agents suitable for delivery to a cell in accordance with some embodiments include paramagnetic species for use in MRI imaging, echogenic entities for use in ultrasound imaging, fluorescent entities for use in fluorescence imaging (including quantum dots), and light-active entities for use in optical imaging. A suitable species for MRI imaging is a gadolinium complex of diethylenetriamine pentacetic acid (DTPA). For positron emission tomography (PET), ¹⁸F or ¹¹C may be delivered. Other non-limiting examples of reporter molecules are discussed throughout the disclosure.

In an aspect, the disclosure provides a kit for detecting CTCs in a sample. In embodiments, the kit comprises an antibody to at least one EMT biomarker. The antibody in the kit can be connected to or associated with an imaging agent. In embodiments, the kit can comprise an antibody to at least one EMT biomarker, wherein the antibody is associated a magnetic bead. The magnetic bead may be used for ferromagnetic separation and enrichment of CTCs.

Aspects also relate to methods of predicting responsiveness of a subject to a cancer drug. The methods may comprise determining the level of expression of at least one EMT biomarker in a sample from the subject. The level of expression of at least one EMT biomarker may be used to obtain a gene expression pattern in CTCs for the subject. The methods may further comprise predicting responsiveness of the subject to the cancer drug based on the gene expression pattern obtained. Genome variation in CTCs from the subject may also be determined.

Also provided are methods of providing a cancer prognosis to a subject. The methods may comprise determining the level of expression of at least one EMT biomarker in a sample from the subject. The level of expression of at least one EMT biomarker may be used to determine the number of CTCs in the sample. The CTCs may be captured using at least one EMT biomarker. The level of expression of at least one EMT biomarker may be used to determine a gene expression pattern in the CTCs for the subject. A prognosis may be provided to the subject based on the gene expression pattern obtained.

Also provided are methods for following the progress of cancer in a subject. The methods may comprise determining the level of expression of at least one EMT biomarker in samples from the subject at a first and a second time, and comparing the first and second levels of expression. The level of expression of at least one EMT biomarker in the sample may be determined over time, such as following initiation of a new cancer therapy. The level of expression of at least one EMT biomarker in the sample may be used to determine the number or amount of CTCs. An increase between the first and second levels may indicate progression of the cancer. A decrease between the first and second levels may indicate remission or response of the cancer to the therapy. No difference between the first and second levels may indicate arrest or stability in the progression of the cancer.

Also provided are methods of screening for cancer in a subject. The methods may comprise determining the level of expression of at least one EMT biomarker in a sample from the subject. The level of expression of at least one EMT biomarker may be used to determine the amount or number of CTCs in the subject. The level of expression of at least one EMT biomarker may be compared to a normal or control sample. An increased level of at least one EMT biomarker may indicate presence of cancer in the subject.

Also provided are methods of arresting cell growth or inducing cell death of a cancer cell expressing an EMT biomarker. The methods include contacting the cancer cell with a conjugate capable of mediating intracellular delivery of an agent, such as the antibodies to EMT markers described herein. The agent is capable of arresting or attenuating the growth of the cell or inducing cell death through any mechanism after agent internalization. The cancer cell may be contacted with the conjugate in vitro, in vivo, or ex vivo. These methods may be useful in treating cancer by directly targeting cancer cells expressing an EMT biomarker for delivery of agents capable of decreasing or arresting cell growth or inducing cell death.

The disclosure also provides for targeted therapeutic methods and molecules that comprise an anti-cancer agent linked to a binding agent that targets at least one EMT as described herein. In some embodiments the link between the anti-cancer agent and the binding agent is a covalent bond. In some embodiments the link is formed by strong electrostatic interactions (hydrogen bonds, hydrophilic/hydrophobic interaction, or oppositely charged moieties, and the like). Any anti-cancer agent can be used in such molecules and therapeutic methods, and can be selected by one of skill in the art based on the type of cancer to be treated, the progress/stage of the cancer, potential adverse drug interactions, dosage requirements, administration schedule, and the like.

### EXAMPLES

### Example 1

### Materials and Methods

Plasmids and Cell Culture.

The minigene used (pRIIIc1²) was previously described (S. Oltean et al., Proc Natl Acad Sci USA 2006, 103, 14116, incorporated herein by reference in its entirety). All cell lines were cultured in low glucose DMEM (Invitrogen) with 10% FBS and 15 IJg/mL blasticidin. Single cell progenies were isolated from a population of AT3 cells stably transfected with pRIIIc1² minigene by limiting dilution to produce a concentration of 1 cell/10 wells and plated on 96-well plates. Cells were counted using a hemocytometer to obtain an initial concentration of 1×10⁵ cells/mL. Through a series of progressive dilutions a final concentration of 1 cell/mL was obtained and 100 IJI were pipetted in each well of three 96-well plates. All wells were monitored through bright field microscopy, those appearing to contain more than one cell were excluded, and those containing single cells were further cultured into 25 mL flasks. 16 of an expected 27 clones were obtained using this procedure in a first round.

To measure cell population growth rate in vitro, cells were plated at 50,000/well in E-well dishes. Viable cells were counted using Trypan Blue staining at 24, 48, 72, and 96h.

Animals and Tumor Cell Implantation.

Cells were trypsinized, washed, and resuspended in PBS at a final concentration of 3×10⁵ cells/mL, and kept on ice for less than 30 minutes before implantation. Cells (3×10⁵) were injected subcutis in both flanks of Copenhagen 2331 rats (Harlan Labs, Indianapolis, Ind.; 75-90 g, 2 months of age). Animals were continuously monitored for tumor growth. All animal procedures were approved by the Duke University Institutional and Animal Care and Use committee and followed NIH guidelines. Sacrifice curves were compared using a Mantel-Haenszel logrank test. Tumor volume was compared using an unpaired t test. Prism 4.0c for the Macintosh (Graphpad, La Jolla, Calif.) was used for statistical analyses.

Histological Sections and Analysis.

Excised tumors and lungs were washed in PBS at room temperature. Depending on the size of the lungs, they were frozen either together or separately. The tumor sections and the lungs were placed in cryomolds, embedded in optimal-cutting-temperature tissue sectioning medium (Sakura Finetek, Torrance, Calif.), snap-frozen in liquid nitrogen, and stored at 80° C. Slides for fluorescence imaging were prepared as follows: the tissue was incubated for 2-3 h at −20° C. to equilibrate the temperature and then sectioned with a microtome. The sections (15 gm) were placed on glass slides, fixed in 4% (wt/vol) paraformaldehyde for 30 min at room temperature, and rinsed in PBS at room temperature. The slides were mounted with gel/mount media (Biomeda, Foster City, Calif.). The sections were analyzed by using an Olympus (Melville, N.Y.) IX 71 epifluorescence microscope, and images were acquired by using an Olympus DP70 digital camera. Image processing was done with DP Controller software (Olympus). For hematoxylin-eosin staining after fluorescence imaging, the slides were incubated in warm water for 15-20 minutes for the cover slip to come off, slides were dried, and staining was performed according to standard procedure.

RNA Extraction from Tumor Sections.

Sections were fixed in 4% (wt/vol) paraformaldehyde for 5 minutes, rinsed in PBS, and imaged. DsRED+ and DsRED− regions of the sections were marked on the slide. The slide was immersed in warm water for 5 minutes to remove the coverslip and the DsRED+ and DsRED− regions scraped off. RNA isolation was further performed as described before (N. Masuda, T. Ohnishi, S. Kawamoto, M. Monden, K. Okubo, Nucleic Acids Res 1999, 27, 4436, incorporated herein by reference in its entirety). Briefly, samples were treated with proteinase K in digestion buffer containing SDS, and further isolation of RNA was performed using the RNeasy kit (QIAGEN, Valencia, Calif.).

Immunoblots.

Cells were collected from confluent 25 cm² tissues flasks by scraping, washed in PBS, and lysed in sample buffer. Whole cell lysates were serially diluted in sample buffer, fractionated via 7.5% SDS-PAGE, and transferred to PVDF. Membranes were cut in half. The bottom half was probed with anti-β-actin at 1:1000 or 1:5000 (Santa Cruz Biotechnology, CA, 47778) as an internal loading control, while the top half was probed with anti-CD 133 (Santa Cruz Biotechnology, CA, 30219) at 1:200 or anti-CD44 (Santa Cruz Biotechnology, CA, 7946) at 1:200.

Gene Expression Analysis.

Triplicate cultures of AT3-M and AT3-T cells were grown to −60% confluency. Total RNA was isolated using the RNeasy kit (Qiagen, Valencia, Calif.), and triplicate samples were submitted to the Duke Microarray Facility. Gene expression analysis was performed using the R027K rat spotted arrays 3.0 (Operon, Huntsville, Ala.). Bioinformatical analysis of expression differences between AT3-M and AT3-T cells was done using the GeneSpring GX software version 7.3.1 (Agilent Technologies, Durham, N.C.). The data files (representing signals for 26,986 gene probes in all six data points, three for AT3-M and three for AT3-T) were normalized using the feature: per Spot and per Chip—intensity dependent (Iowess) normalization. The resulting gene list was used to determine the significantly differentially expressed genes between AT3-M and AT3-T using the “Filtering on Volcano plot” feature with the following characteristics: (1) Test type: Parametric test, don't assume variances equal; (2) Multiple testing correction: None; (3) Fold Difference: Twofold or greater and a P-value cutoff of 0.05.

Analysis of Human Circulating Tumor Cells.

Patients eligible for the CTC biomarker protocols included (1) men with progressive CRPC, with metastatic progression by PSA (two consecutive rises over nadir separated by >1 week) or radiologic criteria (RECIST or new bone scan lesions), a PSA age ≧5, age ≧18 years; or (2) women with mBC with disease progression or with initiation of a new systemic therapy, who were >18 years of age, and who were at least 7 days from treatment with an anthracycline-containing regimen. Blood (15 mL) was collected from patients and processed within 48 hours at the Duke University CTC lab using the Cell Search System (Veridex, Raritan, N.J.). Veridex profile kits were used, which isolate EpCAM positive cells without additional staining. The isolated cells were either processed immediately or stored overnight in 4% paraformaldehyde and processed the next day. Immunostaining was done on teflon coated slides. Briefly, cells were pipetted into the wells of the slides and left to settle for ˜30 minutes followed by standard immunostaining procedures with careful aspiration to minimize cell loss. An initial ferromagnetic wash using a benchtop magnet was performed to further isolate CTCs, with resuspension of the cell pellet after magnet release 100 uL PBS. Following 4% PFA fixation and permeabilization with PBT (PBS with 2% Triton) and blocking with 10% goat serum for 30 minutes, triple immunostaining was performed using CD45 antibody (AbCam #33533-50) labeled with Alexa 647, cytokeratin (AbD Serotec #MCA 1907HT) labeled with Alexa 555, and Vimentin (BD Biosciences, San Jose, Calif. #550513) labeled with Alexa 488. Nuclear staining with 4′,6-diamidino-2-phenylindole (DAPI) was then performed. A CTC was defined as an intact cell by microscopic examination, containing an intact nucleus and expressing cytokeratin but lacking CD45 staining, using appropriate controls (see Table 1 for antibodies and controls). Human peripheral blood mononuclear cells (PBMCs), obtained by Ficoll purification of buffy coats from normal donors, were kindly provided by Micah Luftig (Duke University, Durham N.C.) and used as control cells for CD45 expression. Linear regression analysis was performed to compare CTC count (standard Cellsearch method) against the proportion of CTCs that co-express vimentin. Goodness of fit was tested by analysis of variance.

TABLE 1
EMT/Stemness Antigens to be assessed in CTCs.

| | | Positive | Negative | Leukocyte | |
|---|---|---|---|---|---|
| Antigen | Product | Control | Control | Expression | Dilution |
| Vimentin | BD Biosciences, | PBMCs, PC-3, | T47D, | Yes | 2:225 |
| | mouse monoclonal | DUl45 | LnCAP | | |
| | IgG1 | | | | |
| N-cadherin | DAKO, mouse | Sarcoma, rat | DU145, | No | 4:225 |
| | monoclonal IgG1, | brain, PC-3 | T47D, mock | | |
| | 6G11 | | | | |
| Cytokeratin | AbD Serotec, | T47D, | PC-3, | No | 2:45 |
| (pan) | mouse monoclonal | DUl45 | PBMCs | | |
| | IgG1, MCAI907HT, | | | | |
| | clone AEI/AE3 | | | | |
| CD45 | Invitrogen, | PBMC | PC-3, | Yes | 1:45 |
| | mouse IgG1, | | DUl45 | | |
| | HI30, MHCD4500 | | | | |
| CD133 | Santa Cruz mouse | CaCo-2 | Mock | Variable | 4:225 |
| | monoclonal IgG, | colon cancer | | | |
| | sc-130127 | cells | | | |

The slides were mounted with gel/mount media (Biomeda, Foster City, Calif.). The slides were analyzed with an Olympus (Melville, N.Y.) IX 71 epifluorescence microscope, and images were acquired using an Olympus DP70 digital camera. Image processing was done with DP controller software (Olympus). All fields were analysed, with each cytokeratin positive nucleated cell that was CD45 negative being counted as a CTC. Positive control cells for each antibody included PC-3 cells for vimentin, peripheral blood mononuclear cells (PBMCs) for CD45, and T47D breast cancer cell lines for cytokeratin. A similar volume of reaction mix without antibody was used for negative controls.

Media exchange experiments. The cells of AT3-T or AT3-M clones were plated at a concentration of 150,000 cells/2 mL of media in 6-well plates and allowed to incubate for 24 h. The conditioned media was then filtered using a 0.22 μm filter, and then immediately allowed to incubate with cells of the other clone, which was plated at the same concentration and had its media aspirated and cells washed with 2 mL of PBS. All cells with media replaced were incubated for 72 h, and phase and epifluorescent microscopy was used to monitor cell phenotypes 24, 48, and 72 h after treatment. Control plates, in which media was conditioned, cells washed with PBS and media added back to the same cells, were also used.

Scratch-Wound Assay.

Cells were plated and left to grow to nearly 100% confluency in 6-well dishes. A wound was simulated by scratching the cells with a sterile 200 IJI pipette tip. The wells were washed twice with PBS and fresh media added. Pictures were taken in the same marked spot at 0, 24, and 48 h. Percent migration was calculated as (width at 0 h−width at 24 or 48 h) 1 width at 0 h×100. Relative migration was compared using two-way analysis of variance via Prism 4.0c for the Macintosh (Graphpad, La Jolla, Calif.).

Matrigel Assay.

Matrigel assay was performed per manufacturer's indications (BO Biosciences). Briefly, after rehydration, 2×10⁵ cells were plated either in the control or in the matrigel-coated inserts and incubated for 22 h. Following incubation, the non-invading cells from the upper-part of the inserts were removed using cotton-tipped swabs. The cells from the lower part of the membrane were stained with hematoxylin-eosin, membranes were removed, placed on a slide and observed under the microscope.

Immunohistochemical (IHC) Analysis of Metastases.

Under the same informed consent protocol as the analysis of human circulating tumor cells described above, men undergoing CTC collection additionally consented to have a radiologic-guided metastatic biopsy for analysis of biomarker expression by IHC. Samples were obtained through core needle biopsies during light sedation, and immediately formalin-fixed and paraffin embedded. For analysis, slides were deparaffinized, rehydrated, and endogenous peroxidase was inactivated for 30 min. in 0.3% H₂O₂ (hydrogen-peroxide) in methanol. Specific antigen retrieval steps were performed for individual antigens. Three markers were evaluated by IHC: vimentin (M7020, Dako, 1:150; antigen retrieval with pepsin treatment at 37° C. for 15 minutes), cytokeratin cocktail (18-0132, Invitrogen, 1:50 and 349205, BD Biosciences 1:50, antigen retrieval with pepsin treatment at 37° C. for 15 minutes), and CD45 (M0701, Dako, 1:200; antigen retrieval with sodium citrate 10 mM, pH 6.0 at 100° C. for 30 minutes). Primary antibody was incubated for 60 minutes at room temperature. Dako Envision horseradish peroxidase secondary antibody was used for 30 minutes at room temperature and the signal was detected with DAB reagent (Vector kit SK 4100). Slides were counter stained with hematoxylin and eosin and assessed by a trained pathologist for expression using appropriate positive (localized prostate tissue microarray sections) and negative controls (mock antibody) for each marker.

Statistical Analyses.

To determine the significantly differentially expressed genes between AT3-M and AT3-T the GeneSpring GX “Filtering on Volcano plot” feature was used with the following characteristics: (1) Test type: Parametric test, don't assume variances equal; (2) Multiple testing correction: None; (3) Fold Difference: Twofold or greater and a P-value cutoff of 0.05. To compare CTC count (standard Cellsearch® method) against the proportion of CTCs that co-express vimentin, N-cadherin, or CD133, linear regression analysis was performed. Goodness of fit was tested by analysis of variance.

### Example 2

### Isolation of Individual AT3 Clones that Inhabit an Intermediate Phenotypic State

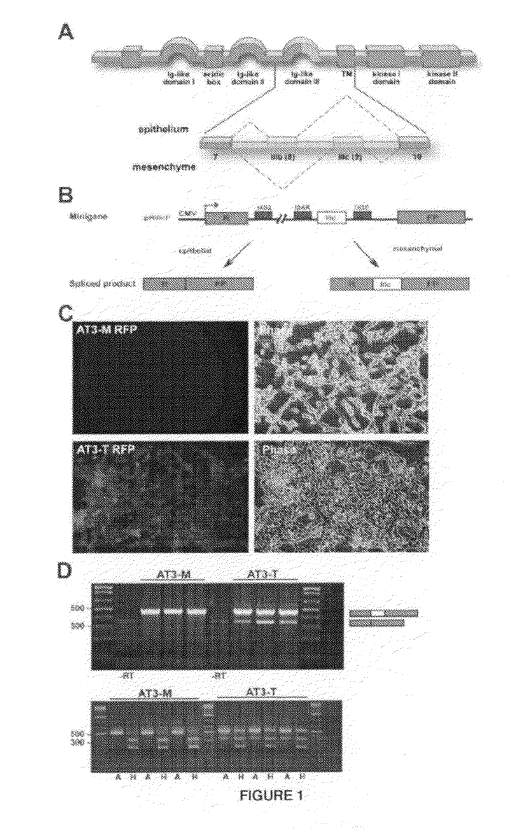

The alternative splicing of FGFR2 transcripts, which produces either FGFR2-IIIb or -IIIc variants in epithelial and mesenchymal cells respectively, is exquisitely regulated (FIG. 1A). In FIG. 1A is a schematic representation of the IIIb and IIIc alternatively spliced isoforms of FGFR2. FGFR2 contains an extracellular domain (with three IgG-like domains), a transmembrane domain (TM), and two intracellular tyrosine kinase domains. The IIIb isoform is found in epithelial cells while the IIIc isoform in mesenchymal cells. Exons IIIb and IIIc are regulated coordinately to provide mutually exclusive expression of the two isoforms and transcripts including both exons are destabilized by nonsense-mediated decay. We have previously used FGFR2 alternative splicing reporters, in particular constructs that measure the epithelial-specific silencing of exon IIIc (e.g., pRIIIc1² in FIG. 1B), to report on the phenotypic state of cells in vitro and in vivo. In FIG. 1B is a schematic of the pRIIIc1² minigene and the fluorescence read-out. The minigene contains the DsRED open reading frame interrupted by exon IIIc and flanking introns of the FGFR2 gene. In epithelial cells exon IIIc is skipped, DsRED open reading frame is formed and results in fluorescence signal. In mesenchymal cells, exon IIIc is included and the DsRED open reading frame is disrupted, resulting in low or close-to-background fluorescence signal. The pRIIIc1² splicing reporter, which produces a variant red fluorescence protein (DsRED) when exon IIIc is silenced, revealed MET in primary tumors derived from AT3 cells implanted in the flanks of Copenhagen white rats. While most tumors contained MET foci, each tumor had very few foci and these were not randomly distributed but rather were associated with collagenous stroma. In contrast to the low frequency of MET in primary tumors, a high incidence of MET among lung metastases in these animals was observed, suggesting an unexpected association between the more epithelial phenotype and aggressive behavior. These studies could not ascertain whether the epithelial-like AT3 cells found in the lungs had undergone MET in the primary tumors or during the process of metastasis.

In an attempt to find post-MET cells in vitro, limiting dilution was used to obtain clones from AT3 cells stably transfected with the pRIIIc1² reporter. A total of 16 clones of a maximum calculated recovery of 27 were obtained, which is ˜60% cloning efficiency. Eleven of these sixteen clones expressed RIIIc1² transcripts (italicized in Table 2), and of these, eight expressed DsRED (Table 2). Some of the clones had an epithelial-like morphology (cells with cobblestone appearance and adherent to each other), while others had a mesenchymal-like morphology (spindle-shaped), as well as clones that displayed a mixed phenotype. It is important to note that given the high cloning efficiency and the high frequency of DsRED+ clones, it is highly unlikely for these epithelial-like clones to come from a very small population within the parental AT3 cells. Rather, the process of subcloning induced a phenotypic transition in a significant number of the AT3 cells.

TABLE 2
Properties of AT3 clones.

| | | | Detection of | |
|---|---|---|---|---|
| | | | exon IIIc skipping | FGFR2 |
| AT3 | Cellular | DsRED | among RIIIcl3 | transcipts |
| Clones | morphology³ | expression² | transcripts¹ | detected³ |
| 1 | Epithelial | High | + | IIIc |
| 2 | Epithelial | High | + | IIIc > IIIb |
| 3 | Epithelial | Low | ND | IIIc > IIIb |
| 4 | Epithelial | Low | ND | IIIc |
| 5 | Epithelial | High | + | IIIc > IIIb |
| 6 | Mesenchymal | Low | ND | IIIc |
| 7 | Mixed | Low | ND | IIIc |
| 8 | Mixed | High | + | IIIc |
| 9 | Mixed | Low | ND | IIIc |
| 10 | Mixed | High | + | IIIc |
| 11 | Mesenchymal | Low | − | IIIc |
| 12 | Mesenchymal | Low | − | IIIc |
| 13 | Epithelial | High | −1 | IIIc > IIIb |
| 14 | Epithelial | Low | − | IIIc |
| 15 | Epithelial | High | + | IIIc |
| 16 | Mixed | High | −2 | IIIc |

¹See FIG. 1C. A “+” indicates detection of RIIIcl² transcripts missing exon IIIc, a “−” all RIIIcl² transcripts include exon IIIc, ND means that no RIIIcl² transcripts were detected.
²Determined by epifluorescence microscopy (high is defined as fluorescence above background of naive AT3 cells and low undistinguishable from the same cells).
³Discussed further herein and illustrated in FIG. 1C.

All of the clones obtained by limiting dilution were analyzed to determine the splicing status of RIIc1² and endogenous FGFR2 transcripts. We could not detect exon IIIc skipping among pRIIIc1² transcripts or any evidence of exon IIIb inclusion among endogenous FGFR2 transcripts in clones with a mesenchymal-like morphology (FIG. 1C and Table 2). FIG. 1C shows RT-PCR analysis of the reporter (upper panel) and endogenous FGFR2 (lower panel). Primers used for the reporter are designed in the DsRED regions flanking exon IIIc. RT-PCR shows a higher percentage of the skipped product in clone AT3-T compared to clone AT3M. Reactions that did not include RT (-RT) reveal a contaminating product that is out-competed by the presence of a bona fide cDNA template (AT3-M lanes). Since exons IIIb and IIIc differ in size by only 3 nucleotides, analysis of the presence of IIIb or IIIc exons in FGFR2 gene was done by using primers in the flanking exons and specific restriction digestion of the resulting RT-PCR products. Exon IIIb is digested by Aval (A) and IIIc by HincIII (H). There is a higher percentage of exon IIIb in clone AT3-T. The RT-PCR are replicates from three different cultures of the two clones. These clones did not express detectable levels of DsRED (FIG. 1D and Table 2). FIG. 1D shows epifluorescence and phase-contrast pictures of clones AT3-M and AT3-T shows the difference in fluorescence intensity and morphology between the two clones. Epifluorescence pictures were taken at the same exposure. All pictures were acquired at 200× magnification. While the skipping of exon IIIc among pRIIIc1² transcripts from epithelial-like clones could be expected, the observation that all of these clones both skipped and included exon IIIc was unexpected (FIG. 1C, Table 2 and data not shown). Analysis of endogenous FGFR2 transcripts revealed that four of the clones with epithelial morphology and DsRED expression had clear evidence of coexpression of both IIIb and IIIc isoforms (Table 2, and FIGS. 1C and 1D). As shown in FIG. 1, AT3-T cells expressed epithelial and mesenchymal isoforms of FGFR2. The expression of DsRED in all the cells suggested that each cell in the culture was expressing both isoforms (FIG. 1C).

We followed two clones with epithelial morphology, high DsRED levels and co-expression of FGFR2-IIIb and -IIIc transcripts (clone 2 and clone 5 (clone 5 herein AT3-T)) and noted that the phenotypic characteristics described above were stable for over six months. Equally, we followed clone 11 (clone 11 herein AT3-M) and clone 12 for six months, and noted that the mesenchymal morphology, undetectable DsRED expression and exclusive production of FGFR2-IIIc were also stable. We concluded from these observations that AT3 cells were plastic and were coaxed by sub-cloning to populate intermediate phenotypic states, with properties of epithelial and mesenchymal cells.

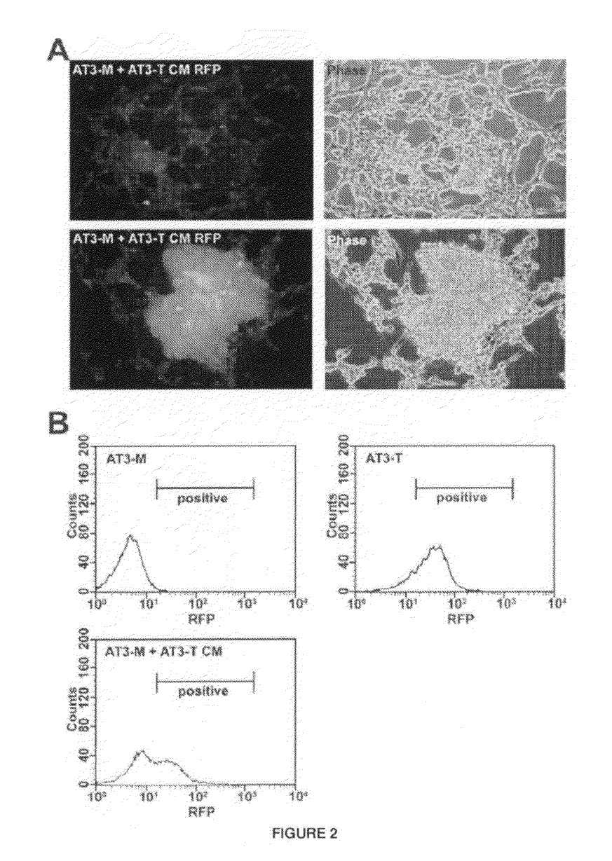

A media exchange experiment was used to investigate whether or not the splicing of RIIIcI² transcripts in the DsRED expressing clones was regulated by soluble factors. Media conditioned by DsRED expressing clones (clone 5 in Table 2) was filtered and added to DsRED negative clones (clone 11 in Table 2). DsRED+ cells were observed among DsRED-cells incubated with DsRED+ conditioned media (FIG. 2). FIG. 2A shows examples of clusters of DsRED positive cells formed by AT3-M cells upon treatment with conditioned media from clone AT3-T. Media was conditioned for 24 h, filtered and added on AT3-M cells. Pictures (acquired at 200×) are taken 48 h following media exchange. FIG. 2B shows results from flow cytometry analysis of the same experiment. Left upper panel represents clone AT3-M conditioned with media from the same clone, as a negative control. Right upper panel represents clone AT3-T, which is DsRED positive. The lower panel represents clone AT3-M 48 h after conditioned media from clone AT3-T was added. Different lots of fetal bovine serum caused variation in this effect. This effect was quantified by flow cytometry and these data suggested that about half of the DsRED− cells were induced to express DsRED at levels equivalent to those seen in DsRED+ cells (FIG. 2). The changes observed were not due to prolonged culture of the cells in the same wells because conditioned media from a separate DsRED− culture did not induce DsRED expression. As shown in FIG. 2, AT3-T conditioned media induced AT3-M cells to express DsRED. These observations suggest that soluble factors secreted by the DsRED+ clones or dilution of factors extant in the DsRED− conditioned media may contribute to plasticity.

### Example 3

### AT3-M and AT3-T Cells are Tumorogenic

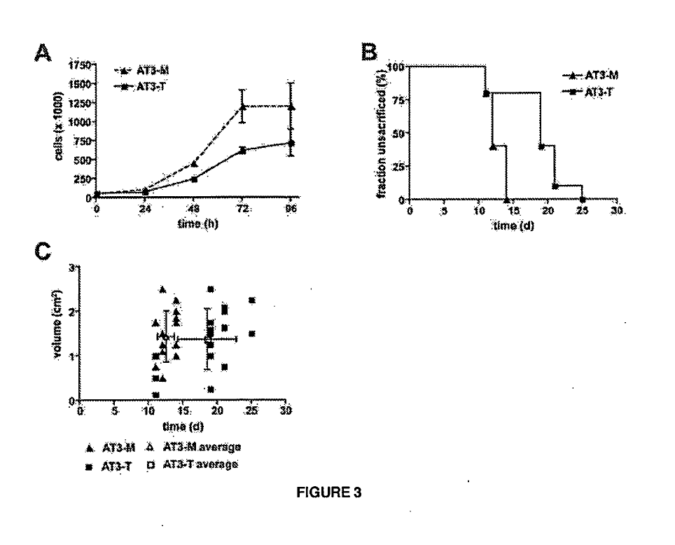

The initial characterization of the AT3-T revealed that these transitional cells grew slower and reached a lower confluent density than the AT3-M (FIG. 3A). FIG. 3A shows growth curves for clones AT3-T and AT3-M. Cells were plated at 0 h time-point, trypsinized, and counted at the indicated times. Data are the mean±S.D. (n=3). To investigate their growth in vivo AT3-M and AT3-T cells were co-transfected with pGint a plasmid that expresses EGFP (herein GFP) in both mesenchymal and epithelial cells, and sorted stable populations of each cell line using flow cytometry for uniform GFP intensity. The GFP expressing cells maintained the morphological characteristics, the differential DsRED expression, and the differences in the splicing of pRIIIc1² and FGFR2 transcripts first observed after sub-cloning.

We injected 3×10⁵ GFP-expressing AT3-T or AT3-M cells subcutis in both flanks of Copenhagen white 2331 male rats. All of the animals developed bilateral tumors, indicating that both AT3-M and AT3-T cells were highly tumorogenic in these syngeneic rats. As a humane endpoint, rats were sacrificed when tumor length estimated by palpation reached 1 cm. The in vivo growth curves for the AT3-M and AT3-T tumors were significantly different, as determined by a logrank test (p=0.0020; FIG. 3B). FIG. 3B is a sacrifice curve for rats injected with AT3-M or AT3-T cells. FIG. 3C shows comparison of tumor volumes resulting from AT3-T and AT3-M injection. The Y-axis represents tumor volumes at the time of sacrifice of the animals and the X-axis days from the time of implantation to the time of sacrifice. Average tumor volumes and average days until sacrifice are represented with S.D. bars. Some points represent more than one tumor with the same volume on the same day. Tumor volume was measured (FIG. 3C) and although most AT3-T animals were sacrificed later, there was no significant difference in tumor size (p=0.76). As shown in FIG. 3, AT3-T cells grew more slowly than the mesenchymal-like AT3-M cells in vitro and in vivo, but both were equally tumorogenic. We concluded that whereas AT3-T cells grew more slowly in vitro and in vivo relative to their more mesenchymal siblings, these transitional cells were capable of forming tumors.

### Example 4

### Both AT3-M and AT3-T are Plastic

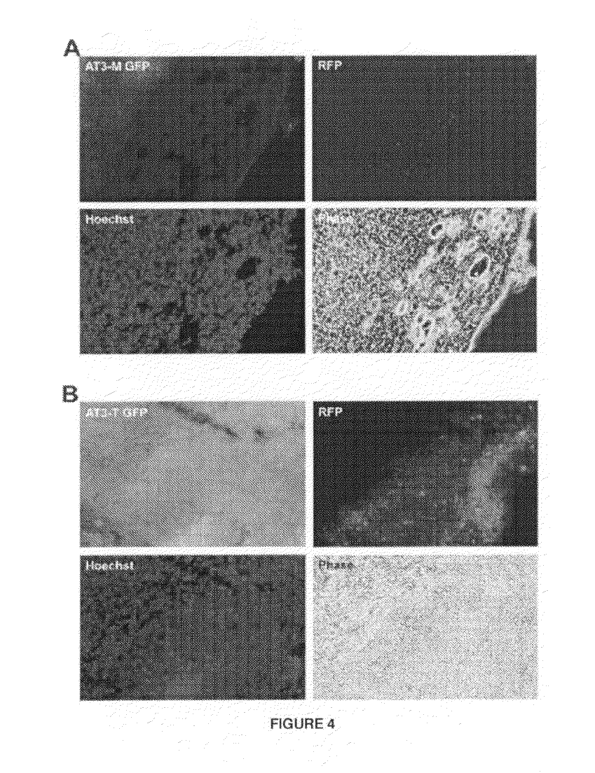

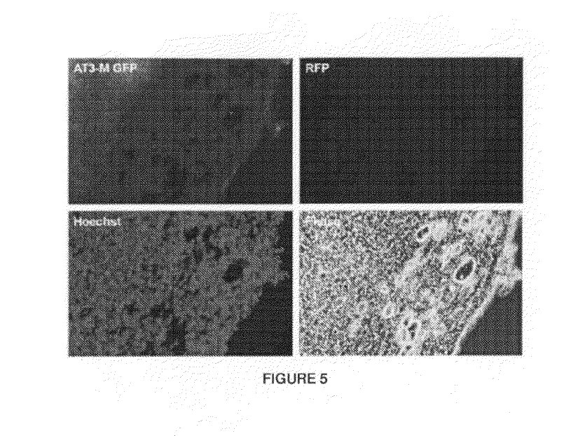

Since the implanted AT3-M and AT3-T cells could be tracked by GFP expression, and epithelial character could be interrogated by DsRED expression, the plasticity of the tumors were able to investigated. The overwhelming majority of cells in AT3-M tumors expressed GFP but not DsRED (FIG. 4A). As shown in FIG. 4, tumors from both AT3-T and AT3-M clones have evidence of plasticity. FIG. 4A shows representative example of cells that express both RFP and GFP at the periphery of an AT3-M tumor stably transfected with Gint and pRIIIc1² reporters. Pictures were taken at 200× magnification. To compensate for a low RFP signal, the color curve of the entire picture was adjusted. Nonetheless, groups of cells were observed expressing both GFP and DsRED in many AT3-M tumor sections, especially near the tumor capsule, (FIG. 4A; see also FIG. 5). FIG. 5 shows a representative example of cells that express both RFP and GFP at the periphery of an AT3-M tumor stably transfected with Gint and pRIIIcI² reporters. Pictures were taken at 200× magnification. In this version, overall RFP signal was not adjusted via color curve after the image was captured. RFP positive cells were clearly above background level.

Many sections from AT3-T tumors co-expressed GFP and DsRED; however, large areas were observed that expressed GFP but not DsRED in all 64 sections surveyed (FIG. 4B). FIG. 4B shows representative example of a section from an AT3-T tumor stably transfected with GFP and pRIIIc1² reporters. Pictures were taken at 200× magnification. RNA extracted from these regions of AT3-T tumors confirmed the presence of the pRIIIc1² transcripts. Both AT3-T and AT3-M cells were plastic and produced tumors with cells that displayed a range of epithelial-mesenchymal properties.

### Example 5

### AT3-T Cells are Motile In Vitro and Metastatic In Vivo

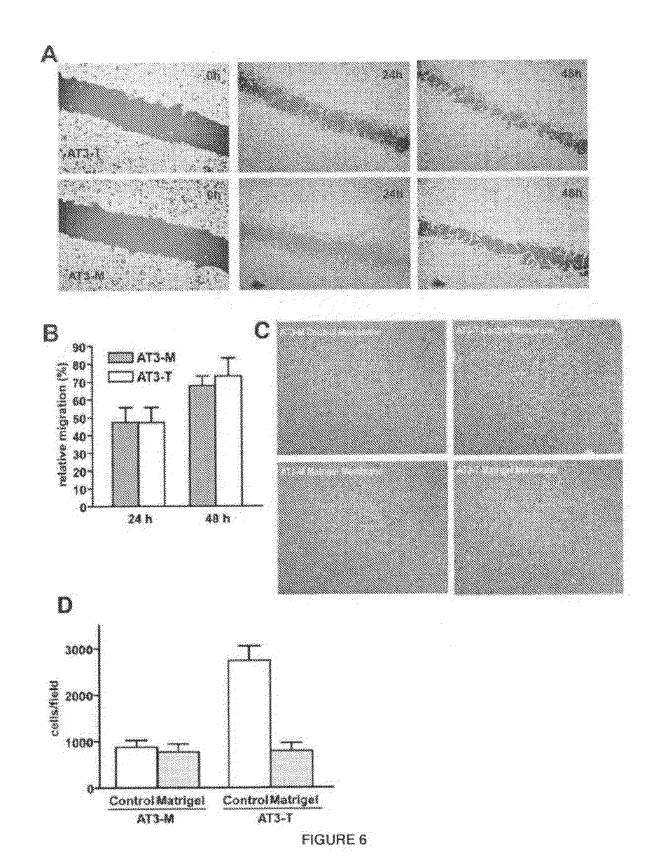

Comparison of AT3-T and AT3-M mobility and invasive potential was performed in culture. Motility was measured in culture by a “wound closure” assay, and no significant motility difference (p=0.59) was found between cell lines 24 and 48 hours after a scratch-wound had been made in the cultures (FIG. 6). FIG. 6A shows representative pictures for the scratch-wound assay (experiment done in triplicate for each clone). Pictures were taken at 40× magnification. FIG. 6B shows quantification of migration as explained in Methods. Mean and SO values were derived from triplicate experiments. FIG. 6C shows invasion assay using Matrigel coated membranes. Representative pictures of each clone and for both control membranes and Matrigel-coated membranes (n=5). Cells were stained with hematoxylin-eosin. Pictures were taken at 40× magnification. FIG. 6D shows quantification of invasion assay results. Mean and SD values were derived from five individual experiments. To gauge invasive properties of the cells we measured the number of cells traversing through Matrigel membranes in a 22-hour period. The same number of AT3-T and AT3M cells was observed on the Matrigel membranes suggesting that the two cell lines were equally capable of invading this membrane (FIG. 6). While a higher number of cells from clone AT3-T were observed on the control membrane compared to clone AT3-M, these studies nevertheless indicated that the more epithelial AT3-T cells had similar motility and invasive potential as the AT3-M cells. As shown in FIG. 6, AT3-M and AT3-T cells exhibited similar migration in vitro.

In order to assess invasiveness in vivo lungs from the twenty animals harboring AT3-M and AT3-T tumors were examined for presence of metastatic foci. No macroscopic metastatic nodules were observed in any of the lungs, which was likely due to the sacrificing protocol used on the animals when the tumors reached a specified size instead of using survival as the end-point. The GFP expression from the Gint reporter was examined to evaluate the presence of micrometastases by epifluorescence microscopy. To assure a comprehensive evaluation, 7-8 equally spaced sections from each lung were surveyed (total of 150 sections for each clone). The presence of metastatic foci was determined by GFP fluorescence, followed by counter-staining of the sections with hematoxylineosin (FIG. 7). FIG. 7A shows (upper panel) an example of a section exhibiting the expected pattern for clone AT3-T (i.e. GFP+, DsRED+) in a metastatic focus, and (lower panel) an example of a section exhibiting a plastic pattern for clone AT3-T (i.e. GFP+, DsRED−) in a metastatic focus. FIG. 7B shows (upper panel) an example of a section exhibiting the expected pattern for clone AT3-M (i.e. GFP+, DsRED−) in a metastatic focus, and (lower panel) an example of a section exhibiting a plastic pattern for clone AT3-M (i.e. GFP+, DsRED+) in a metastatic focus. As shown in FIG. 7, metastatic foci in lungs from animals with tumors from either AT3Tor AT3-M clones (stably transfected with GFP and pRIIIcI² reporters) had evidence of plasticity. Metastatic foci were found in 7 out of 10 lungs for clone AT3-M and 6 out of 10 lungs for clone AT3-T.

Evaluation of the plasticity of the metastatic foci using the combined output of the GFP and DsRED reporters revealed plastic foci (DsRED+ for AT3-M and DsRED− for AT3-T) in the case of both clones: 3 out of 12 for clone AT3-T and 13 out 16 for clone AT3-M (FIG. 7). These studies indicated phenotypic plasticity for the AT3-M cells and suggested it for the AT3-T cells. Importantly, both cell lines were metastatic despite differences in the original epithelial vs. mesenchymal phenotype.

Plasticity and Metastatic Behavior of Cancer Cells.

Both the mesenchymal AT3-M and the more epithelial AT3-T cells metastasized efficiently. The drivers of metastasis, however, may be different in these two cells. The gene expression comparison between the AT3-M and AT3-T clones revealed at least one intriguing possibility: microarray analysis showed a 12-fold increase in the expression of junctional adhesion molecule C (JAM-C) in AT3-T compared to AT3-M, and this was confirmed by RT-PCR and immunoblot analysis. JAMs were present in leukocytes and at the tight junctions of epithelial and endothelial cells and have been shown to be involved in transendothelial migration of monocytes. JAM-C is expressed in several cell lines with high metastatic potential and knock-down of this molecule in the HT1080 human fibrosarcoma line significantly decreases its metastatic properties in vivo. Moreover, JAM-C is also present in the gene sets associated with sternness that had significant overlaps with genes that define clone AT3-T. Therefore clone AT3-T, by over-expression of different adhesion molecules may acquire metastatic capabilities. In addition, the overexpression of the downstream Hedgehog pathway effector GLI3 may be significantly upregulated in the more epithelial and stem cell-like AT3-T cells as compared to the more mesenchymal AT3-M cells. Hedgehog signaling has been linked to EMT, sternness, and metastasis/aggressiveness in several tumor types, and thus differential expression or regulation of developmental programs may underly these phenotypical differences across these cell lines. Increased expression of Patched, a Hedghog pathway component, has been linked to prostate tumors during progression to androgen independence and in circulating tumor cells of men with metastatic castration-resistant prostate cancer.

### Example 6

### AT3-T Cells Display a Stem Cell-Like Gene Expression Signature

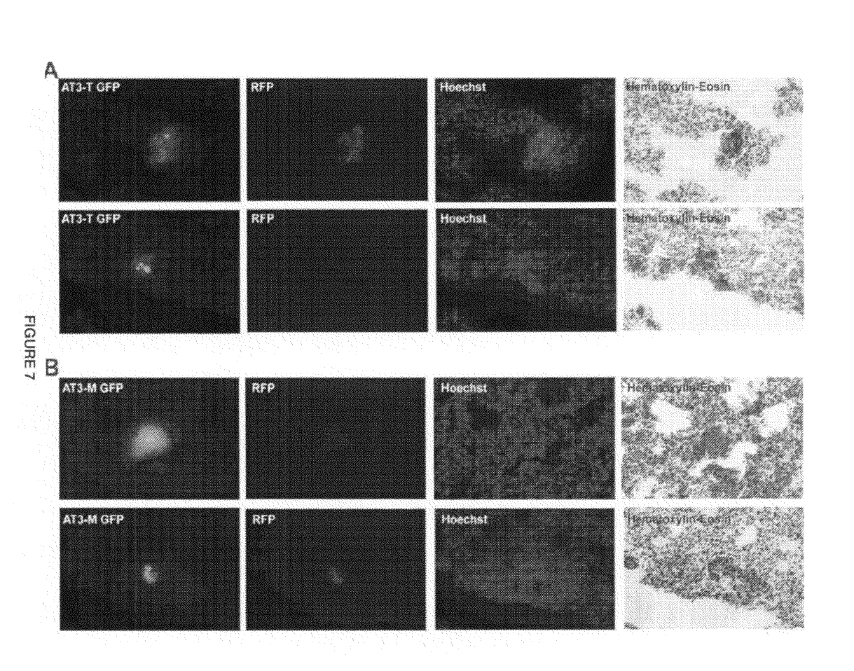

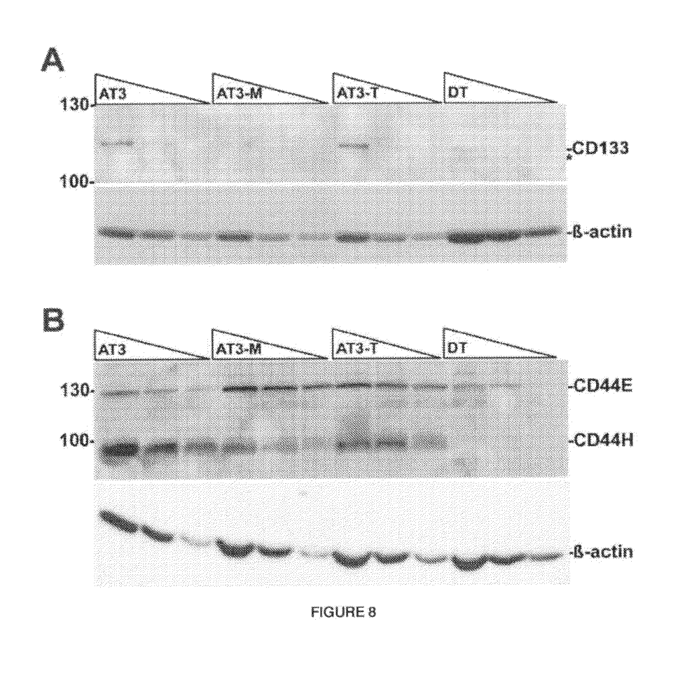

AT3-T cells sometimes formed tight clusters resembling protospheres. While sphere formation is not an exclusive property of stem cells, it has been associated with sternness in many different systems. Given these observations and the high tumorogenicity of AT3-T and AT3-M cells, they were tested for the expression of markers associated with cancer stem-like cells. Also included were the parental AT3 cells and another Dunning tumor cell line, DT cells, which display epithelial markers and are only weakly tumorogenic in Copenhagen white rats. The DT cells expressed very low levels of CD44 and CD133, which are associated with highly malignant cancer stem-like cells (FIG. 8). CD133 was detectable in DT lysates only when four fold more lysate was loaded. The mesenchymal-like AT3 cells expressed much higher levels of both CD44 and CD133 than the DT cells (note that the lanes for the DT samples are overloaded in FIG. 8A), which is consistent with recent reports that EMT induces sternness in mammary epithelial carcinoma cells. FIG. 8A shows a membrane with serial twofold dilutions of whole cell lysates was cut in half and immunoblotted for CD133 (upper panel) or β-actin (lower panel). Size markers are in kDa. A faster migrating CD133 band repeatably detected only in DT lysates is marked (*), suggesting possible post-translational regulation. FIG. 8B shows a membrane with serial twofold dilutions of whole cell lysates was cut in half and immunoblotted for CD44 (upper panel) or β-actin (lower panel). Representative blots from two independent sets of lysates are shown. AT3-T expressed CD44 and CD133. Interestingly, the AT3-T cells expressed overall higher levels of CD44 and CD133 than the more mesenchymal AT3-M. Moreover, AT3-T cells expressed a higher ratio of CD44H to CD44E when compared to AT3-M cells. The CD44H isoform has been associated with malignancy while CD44E is not. This suggests a more complex relationship between epithelial transitions and acquisition of stem cell-like properties. Consistent with expression of stem-like markers, both AT3-M and AT3-T cells formed colonies in soft agar and tumors when injected into Copenhagen white rats, and these tumors led to extensive metastases similar to parental AT3 cells (FIG. 3B).

To further explore these connections between transitions and sternness, global gene expression in AT3-M and AT3-T cells was compared. This analysis showed that 422 genes were differentially expressed (≧2-fold; p-value <0.05) in these two cells (Table 3). Many of the genes that were upregulated in AT3-T relative to AT3-M were preferentially expressed in epithelial cells and vice versa for those preferentially expressed in mesenchymal cells (Table 4). There were exceptions to this, however. Expression of the gene disintegrin-like and metalloprotease was consistent with a mesenchymal phenotype, but this mRNA level was 4-fold higher in AT3-T compared to AT3-M. Integrin β-4, normally associated with epithelial-like cells, was expressed 3-fold lower in AT3-T compared to AT3-M. These observations were consistent with the characterization of AT3-T cells as displaying more epithelial features than AT3-M cells and as populating an intermediate phenotypic state.

TABLE 3

| x Fold change | Gene Symbol | Gene Symbol |
|---|---|---|
| (AT3-T/AT3-M) | (Human) | (Rat) |
| 0.00771 | P2RX5 | P2rx5 |
| 0.011 | CCNB1lP1 | #N/A |
| 0.0296 | STRA6 | Stra6 |
| 0.0327 | G0S2 | G0s2 |
| 0.0835 | SERPINF1 | Serpinf1 |
| 0.101 | GSTA1 | #N/A |
| 0.107 | RSNL2 | Clip4 |
| 0.115 | ADAMTS7 | #N/A |
| 0.134 | GZMB | #N/A |
| 0.137 | SPON2 | #N/A |
| 0.156 | MMP3 | #N/A |
| 0.191 | ATP8A1 | #N/A |
| 0.197 | EVPL | Evpl |
| 0.21 | LGALS3BP | Lgals3bp |
| 0.216 | SERPINB2 | Serpinb2 |
| 0.219 | NETO2 | Neto2 |
| 0.223 | PTX3 | #N/A |
| 0.23 | SERPINB7 | Serpinb7 |
| 0.233 | RASIP1 | #N/A |
| 0.235 | OMD | #N/A |
| 0.239 | HLA-G | #N/A |
| 0.239 | HLA-A | #N/A |
| 0.247 | CD97 | Cd97 |
| 0.251 | GJA4 | Gja4 |
| 0.254 | DSU | #N/A |
| 0.257 | MGLL | Mgll |
| 0.261 | SPHK1 | #N/A |
| 0.268 | HRBL | Zcwpw1 |
| 0.268 | ZCWPW1 | Zcwpw1 |
| 0.27 | ENPP3 | Enpp3 |
| 0.275 | PTGS1 | Ptgs1 |
| 0.278 | RAMP1 | Ramp1 |
| 0.281 | DHRS3 | Dhrs3 |
| 0.282 | FAM117A | Fam117a |
| 0.284 | TUBB2A///TUBB2B | Tubb2b |
| 0.284 | TUBB2B | Tubb2b |
| 0.285 | C10orf10 | LOC500300 |
| 0.289 | SYTL2 | #N/A |
| 0.291 | SLC39A4 | Slc39a4 |
| 0.292 | CHRD | Chrd |
| 0.292 | GIP | Gip |
| 0.293 | CKLF | Cklf |
| 0.294 | PLAU | Plau |
| 0.295 | GUF1 | #N/A |
| 0.307 | CGI-38 | Tppp3 |
| 0.311 | LECT2 | Lect2 |
| 0.318 | NQO2 | #N/A |
| 0.32 | C11orf75 | RGD1309410 |
| 0.324 | DOCK2 | #N/A |
| 0.325 | LGALS2 | #N/A |
| 0.326 | CASP4 | Casp1 |
| 0.326 | LTBP4 | Ltbp4 |
| 0.334 | HSPB1 | Hspb1 |
| 0.335 | ITGB4 | Itgb4 |
| 0.34 | BPHL | Bphl |
| 0.341 | FOXF2 | #N/A |
| 0.345 | MYH1 | #N/A |
| 0.345 | SMAD6 | Smad6 |
| 0.348 | TGFB1 | Tgfb1 |
| 0.351 | MMP10 | #N/A |
| 0.363 | MMP9 | Mmp9 |
| 0.363 | COL18A1 | Col18a1 |
| 0.366 | HES1 | #N/A |
| 0.369 | SLC35D2 | #N/A |
| 0.377 | ADORA2B | Adora2b |
| 0.377 | COL3A1 | Col3a1 |
| 0.379 | DPEP2 | Dpep2 |
| 0.382 | GPR153 | Gpr153_predicted |
| 0.383 | LOC55908 | #N/A |
| 0.389 | SELPLG | #N/A |
| 0.394 | P2RX1 | Atp2a3 |
| 0.394 | ATP2A3 | Atp2a3 |
| 0.394 | ADD3 | Add3 |
| 0.395 | TSPAN9 | Tspan9 |
| 0.399 | LOC54103 | #N/A |
| 0.4 | BFSP2 | #N/A |
| 0.4 | FLJ14213 | RGD1309969 |
| 0.4 | PGGT1B | Pggt1b |
| 0.401 | HCN2 | Hcn2 |
| 0.403 | C2orf33 | RGD1310230 |
| 0.404 | TMEPAI | #N/A |
| 0.405 | INHA | Inha |
| 0.406 | HPSE | #N/A |
| 0.409 | CRY1 | Cry1 |
| 0.413 | IL3RA | ll3ra |
| 0.413 | CDC42EP1 | #N/A |
| 0.416 | ARG1 | Arg1 |
| 0.417 | MAPK14 | Mapk14 |
| 0.419 | FLJ22028 | #N/A |
| 0.421 | GALR2 | Galr2 |
| 0.422 | TSPAN8 | Tspan8 |
| 0.422 | FAM77C | RGD1561205 |
| 0.422 | USP2 | Usp2 |
| 0.422 | LAMA3 | #N/A |
| 0.424 | CCNE1 | Ccne1 |
| 0.424 | NSF | Nsf |
| 0.428 | ST3GAL5 | St3gal5 |
| 0.429 | SYNJ2 | Synj2 |
| 0.43 | ADA | Ada |
| 0.43 | PCBP3 | Pcbp3 |
| 0.433 | ZNF43 | #N/A |
| 0.433 | C14orf130 | Ubr7 |
| 0.436 | SOS2 | #N/A |
| 0.436 | RASSF3 | #N/A |
| 0.436 | GLMN | Glmn |
| 0.438 | OSR2 | Osr2 |
| 0.44 | AGTPBP1 | Agtpbp1 |
| 0.444 | DBNDD2 | RGD1311642 |
| 0.445 | SGCB | #N/A |
| 0.446 | HBLD2 | Isca1 |
| 0.448 | SCARB1 | Scarb1 |
| 0.448 | EVI2A | Evi2a |
| 0.448 | AP4M1 | #N/A |
| 0.451 | IGF2BP3 | #N/A |
| 0.452 | FLJ10404 | Ddx41 |
| 0.454 | TGFB2 | Tgfb2 |
| 0.459 | PASK | Pask |
| 0.461 | C19orf37 | Zfp428 |
| 0.462 | BMP1 | Bmp1 |
| 0.464 | PTPN13 | Ptpn13 |
| 0.47 | PTPRG | #N/A |
| 0.47 | EFNB1 | Efnb1 |
| 0.472 | PER2 | Per2 |
| 0.472 | IRS3L /// LOC442715 | Irs3 |
| 0.472 | HRBL | Irs3 |
| 0.472 | MAP3K3 | Kcnh6 |
| 0.472 | WDR68 | Kcnh6 |
| 0.472 | KCNH6 | Kcnh6 |
| 0.472 | CCDC44 | Kcnh6 |
| 0.473 | CIB2 | Cib2 |
| 0.475 | MPZL1 | Mpzl1 |
| 0.475 | FADS2 | #N/A |
| 0.48 | ZNF185 | #N/A |
| 0.482 | SLC29A1 | Slc29a1 |
| 0.487 | RUNX3 | Runx3 |
| 0.488 | NINJ1 | Ninj1 |
| 0.489 | RASL11B | Rasl11b |
| 0.49 | ECE2 | Ece2 |
| 0.49 | TNNC2 | Tnnc2 |
| 0.491 | WASPIP | Wipf1 |
| 0.492 | FN1 | Fn1 |
| 0.494 | NDE1 | Nde1 |
| 0.494 | CAMK2G | Camk2g |
| 0.495 | CUTL1 | Cux1 |
| 0.495 | ABHD6 | Abhd6 |
| 0.495 | PTPN14 | Ptpn14 |
| 0.497 | FLJ13946 | #N/A |
| 0.498 | BAIAP2 | Baiap2 |
| 0.499 | MSL3L1 | Msl3l1 |
| 0.499 | DYNLT1 | Dynlt1 |
| 0.499 | GSTM3 | Gstm5 |
| 2 | CHES1 | Foxn3 |
| 2.004 | AQR | Agr /// Znf770 |
| 2.006 | EPN1 | Epn1 |
| 2.011 | PPBP | Ppbp |
| 2.019 | SLC35D1 | #N/A |
| 2.022 | PTPRC | #N/A |
| 2.031 | USP47 | Usp47 |
| 2.041 | DHX29 | #N/A |
| 2.047 | HMOX1 | #N/A |
| 2.05 | CAV1 | Cav1 |
| 2.053 | BUB1B | Bub1b |
| 2.069 | KCNIP4 | #N/A |
| 2.072 | — | #N/A |
| 2.072 | ADAM10 | #N/A |
| 2.073 | KIAA1155 | #N/A |
| 2.074 | PSTPIP2 | #N/A |
| 2.083 | MAML1 | #N/A |
| 2.084 | RAB32 | #N/A |
| 2.089 | FAM111A | #N/A |
| 2.095 | ATRNL1 | #N/A |
| 2.101 | PPIC | Ppic |
| 2.101 | CHD4 | Chd4 |
| 2.109 | IDE | Ide |
| 2.117 | PITPNM3 | #N/A |
| 2.121 | NFE2L1 | Nfe2l1 |
| 2.121 | MFSD1 | #N/A |
| 2.133 | KITLG | Kitlg |
| 2.161 | ING3 | Ing3 |
| 2.167 | CD24 | #N/A |
| 2.169 | IDS | #N/A |
| 2.177 | MGC3196 | LOC686289 /// |
| | | LOC690285 |
| 2.185 | FBXL11 | Fbxl11 |
| 2.185 | — | Fbxl11 |
| 2.191 | ZC3H12A | #N/A |
| 2.195 | RKHD2 | #N/A |
| 2.201 | LAMC2 | Lamc2 |
| 2.217 | KIF11 | Kif11 |
| 2.242 | SNAPC5 | Snapc5 |
| 2.252 | THRAP3 | #N/A |
| 2.261 | HS6ST1 | #N/A |
| 2.264 | OXCT1 | #N/A |
| 2.266 | TEK | #N/A |
| 2.268 | HIST2H4///H4/o/// | #N/A |
| | LOC648164 | |
| 2.271 | TMF1 | Tmf1 |
| 2.273 | ZBTB7B | Zbtb7b |
| 2.274 | CAMSAP1L1 | RGD1310950 |
| 2.279 | CYP3A5 | Cyp3ai |
| 2.279 | CYP3A7 | Cyp3a9 |
| 2.279 | CYP3A4 | Cyp3a9 |
| 2.282 | PENK | Penk1 |
| 2.283 | KIAA2010 | Smek1 |
| 2.284 | CHRNA1 | #N/A |
| 2.299 | BAT3 | Bat3 |
| 2.302 | ROM1 | Rom1 |
| 2.306 | HOXB8 | #N/A |
| 2.309 | KLK14 | #N/A |
| 2.31 | SUV39H1 | #N/A |
| 2.315 | LOC440354///BOLA2/// | RGD1564579 |
| | LOC595101 | |
| 2.315 | UBN1 | Ubn1 |
| 2.323 | C1orf103 | #N/A |
| 2.333 | EYA2 | Eya2 |
| 2.347 | MT2A | #N/A |
| 2.353 | KIAA1815 | Ermp1 |
| 2.355 | SETD1B | #N/A |
| 2.369 | MPHOSPH1 | Kif20b |
| 2.38 | EFNA1 | Efna1 |
| 2.392 | ABCF2 | Abcf2 |
| 2.397 | LIMA1 | Lima1 |
| 2.418 | EXTL3 | Extl3 |
| 2.418 | ARL6IP2 | Arl6ip2 |
| 2.442 | GRAMD3 | Gramd3 |
| 2.456 | JARID1A | Jarid1a |
| 2.476 | ARHGEF9 | Arhgef9 |
| 2.485 | CAD | Cad |
| 2.493 | RAI17 | #N/A |
| 2.526 | KIAA0284 | #N/A |
| 2.529 | SGPP1 | Sgpp1 |
| 2.531 | ABCB1 | #N/A |
| 2.531 | ABCB1///ABCB4 | #N/A |
| 2.542 | KIF1C | #N/A |
| 2.553 | KIAA0020 | LOC499339 |
| 2.563 | ADAM15 | Adam15 |
| 2.577 | UBE1 | Uba1 |
| 2.577 | INE1 | Uba1 |
| 2.58 | GRIP2 | Grip2 |
| 2.59 | PPEF1 | #N/A |
| 2.619 | SC65 | Sc65 |
| 2.62 | FER1L3 | #N/A |
| 2.62 | NOC3L | #N/A |
| 2.62 | RBP4 | #N/A |
| 2.645 | SPINK4 | Spink4 |
| 2.653 | ATXN2L | #N/A |
| 2.711 | AHCYL1 | Ahcyl1 |
| 2.723 | TUBB3 | Tubb3 |
| 2.723 | MC1R | Tubb3 |
| 2.729 | AGPAT7 | Lpcat4 |
| 2.749 | HOXC11 | #N/A |
| 2.766 | APH1A | Aph1a |
| 2.785 | CNOT1 | RGD1308009 |
| 2.785 | CSNK2A2 | RGD1308009 |
| 2.794 | STAC | #N/A |
| 2.904 | STAG1 | #N/A |
| 2.942 | MBNL1 | #N/A |
| 2.982 | MNT | Mnt |
| 3.007 | RANBP5 | Ipo5 |
| 3.014 | HERC1 | Herc1 |
| 3.065 | ALDOC | Aldoc |
| 3.122 | KIAA0460 | — |
| 3.174 | FLT3 | #N/A |
| 3.278 | CXCL6 | Cxcl6 |
| 3.366 | GLI3 | #N/A |
| 3.489 | SSR3 | #N/A |
| 3.585 | BCAN | Bcan |
| 3.824 | FKBP10 | Fkbp10 |
| 3.903 | GSTK1 | Gstk1 |
| 3.931 | PSCDBP | #N/A |
| 3.974 | ALCAM | Alcam |
| 4.056 | ADAMTS13 | |
| 4.203 | SPRR2B | #N/A |
| 4.276 | GPR126 | #N/A |
| 5.169 | SULF1 | Sulf1 |
| 5.529 | TFF1 | Tff1 |
| 6.52 | PTN | Ptn |
| 8.591 | MLF1 | Mlf1 |
| 9.012 | THBS2 | Thbs2 |
| 10.79 | HEPH | Heph |
| 12.53 | JAM3 | Jam3 |

TABLE 4
Examples of epithelial or mesenchymal genes in the
expression data analysis of clones AT3-T and AT3-M.

| | x Fold change in |
|---|---|
| Gene name | AT3-T vs. AT3-M |
| Junctional adhesion molecule C | 12.53 |
| Disintegrin-like and metalloprotease | 4.05 |
| Activated leukocyte cell adhesion molecule | 3.97 |
| Tubulin | 2.73 |
| Epithelial protein lost in neoplasm | 2.39 |
| Laminin | 2.20 |
| TGFβ2 | 0.45 |
| MMP9 | 0.36 |
| Collagen, type XVIII | 0.36 |
| MMP10 | 0.35 |
| Integrin β4 | 0.33 |
| TGFβ1 | 0.31 |
| Urokinase plasminogen activator | 0.29 |
| MMP3 | 0.15 |

Two gene sets were assembled: one composed of gene products upregulated in AT3-T (relative to AT3-M) and the second of those downregulated in AT3-T (relative to AT3-M). The two gene sets were compared for overlap with 5,452 gene sets from the Molecular Signature Database collections (Gene Set Enrichment Analysis (GSEA) http://www.broad.mit.edu/gseaf). Analysis of genes over-expressed in AT3-T relative to AT3-M for overlap with 5,452 gene sets from the Molecular Signature Database collections via Gene Set Enrichment Analysis (GSEA) did not show any significant enrichment of sets associated with EMT or MET. In this regard, both AT3-M and AT3-T resembled the mesenchymal-like, parental AT3 line. Among the 15 most significant overlaps for the genes overexpressed in AT3-T there were three sets of genes activated in hematopoetic stem cells (p=3.24×10⁻⁸), neural stem cells (p=3.07×10⁻⁷) and embryonal murine stem cells (p=5.14×10⁻⁶), (Table 5) while among the 20 most significant overlaps for the genes that are relatively downregulated in AT3-T cells were two gene sets associated with development of mature cell types. Expression of the downstream hedgehog pathway effector GL13 was found to be 3.4-fold overexpressed in AT3-T cells compared to AT3-M cells, indicating that regulation of this developmental/stemness pathway in prostate cancer may be tied to the underlying phenotypic state during EMT/MET, similar to what has been reported in other tumors. These data indicated that AT3-T cells have gene expression profiles similar to stem cells, and, in concordance with the analysis of CD44 and CD133 protein expression, suggested that AT3-T cells exist in a more stem cell-like state than the more mesenchymal AT3-M cells.

TABLE 5
GSEA Collections: C1, C3, C2, C5, C4
# overlaps shown: 20
# gene sets in collections: 5452
# genes in comparison (N) 127
# genes in collections (N) 39655

| | # genes in | | # genes in | | |
|---|---|---|---|---|---|
| gene set name | gene set (k) | Description | overlap (k) | k/K | p value |
| TATAAA_V$TATA_O1 | 1333 | Genes with promoter regions | 20 | 0.015 | 8.07E−09 |
| | | [−2 kb, 2 kb] around transcription | | | |
| | | start site containing the motif | | | |
| | | TATAAA which matches annotation | | | |
| | | for TAF<br> TATA | | | |
| STEMCELL_HEMATOPOIET IC_UP | 1452 | Enriched in mouse hematopoietic | 20 | 0.0138 | 3.24E−08 |
| | | stem cells, compared to differen- | | | |
| | | tiated brain and bone marrow cells | | | |
| GNF2_RAP1B | 37 | Neighborhood of RAP1B | 5 | 0.1351 | 1.23E−07 |
| STEMCELL_NEURAL_UP | 1838 | Enriched in mouse neural stem | 21 | 0.0114 | 3.07E−07 |
| | | cells, compared to differentiated | | | |
| | | brain and bone marrow cells | | | |
| module 2 | 383 | Genes in Module_2 | 10 | 0.0261 | 4.34E−07 |
| CTTTGA_V$LEF1_Q2 | 1270 | Genes with promoter regions | 17 | 0.0134 | 5.48E−07 |
| | | [−2 kb, 2 kb] around transcription | | | |
| | | start site containing the motif | | | |
| | | CTTTGA which matches annotation | | | |
| | | for LEF1: lymphoid enhancer- | | | |
| | | binding factor 1 | | | |
| SIGNAL_TRANSDUCTION | 1637 | Genes annotated by the GO term | 19 | 0.0116 | 9.33E−07 |
| | | GO:0007165. The cascade of | | | |
| | | processes by which a signal | | | |
| | | interacts with a receptor, causing | | | |
| | | a change in the level or activity | | | |
| | | of a second messenger or other | | | |
| | | downstream target, and ultimately | | | |
| | | effecting a change in the | | | |
| | | functioning of the cell. | | | |
| module_385 | 28 | Genes in module 385 | 4 | 0.1429 | 1.91E06 |
| V$MYCMAX_O1 | 261 | Genes with promoter regions | 8 | 0.0307 | 1.98E06 |
| | | [−2 kb, 2 kb] around transcription | | | |
| | | start site containing the motif | | | |
| | | NNACCACGTGGTNN which | | | |
| | | matches annotation for MYC: | | | |
| | | v-myc myelocytomatosis viral | | | |
| | | oncogene homolog (avian)<br> | | | |
| | | MAX: MYC associated factor X | | | |
| GGGCGGR_V$SP1_Q6 | 3053 | Genes with promoter regions | 26 | 0.0085 | 2.59E−06 |
| | | [−2 kb, 2 kb] around transcription | | | |
| | | start site containing the motif | | | |
| | | GGGCGGR which matches annotation | | | |
| | | for SP1: Sp1 transcription factor | | | |
| AACTTT_UNKNOWN | 1963 | Genes with promoter regions | 20 | 0.0102 | 3.29E−06 |
| | | [−2 kb, 2 kb] around transcription | | | |
| | | start site containing motif | | | |
| | | AACTTT. Motif does not match | | | |
| | | any known transcription factor | | | |
| V$AP1_C | 281 | Genes with promoter regions | 8 | 0.0285 | 3.38E−06 |
| | | [−2 kb, 2 kb] around transcription | | | |
| | | start site containing the motif | | | |
| | | NTGASTCAG which matches | | | |
| | | annotation for JUN: jun oncogene | | | |
| MEMBRANE_PART | 1673 | Genes annotated by the GO | 18 | 0.0108 | 5.09E−06 |
| | | term GO:0044425. Any constituent | | | |
| | | part of a membrane, a double | | | |
| | | layer of lipid molecules that | | | |
| | | encloses all cells, and, in | | | |
| | | eukaryotes, many organelles; | | | |
| | | may be a single or double lipid | | | |
| | | bilayer; also includes associated | | | |
| | | proteins. | | | |
| STEMCELL_EMBRYONIC_UP | 1344 | Enriched in mouse embryonic stem | 16 | 0.0119 | 5.14E−06 |
| | | cells, compared to differentiated | | | |
| | | brain and bone marrow cells | | | |
| INTRINSIC_TO_MEMBRANE | 1350 | Genes annotated by the GO term | 16 | 0.0119 | 5.43E−06 |
| | | GO:0031224. Located in a membrane | | | |
| | | such that some covalently attached | | | |
| | | portion of the gene product, for | | | |
| | | example part of a peptide sequence | | | |
| | | or some other covalently attached | | | |
| | | moiety such as a GPI anchor, spans | | | |
| | | or is embedded in one or both | | | |
| | | leaflets of the membrane. | | | |
| CELL_SURFACE | 79 | Genes annotated by the GO term | 5 | 0.0633 | 5.58E−06 |
| | | GO:0009986. The external part | | | |
| | | of the cell wall and/or plasma | | | |
| | | membrane. | | | |
| UVC_XPCS_8HR_DN | 408 | Down-regulated at 8 hours following | 9 | 0.0221 | 6.35E−06 |
| | | treatment of XPB/CS fibroblasts | | | |
| | | with 3 J/m\hat{ }2 UVC | | | |
| NOTCH_SIGNALING_PATHWAY | 12 | Genes annotated by the GO term | 3 | 0.25 | 6.86E−06 |
| | | GO:0007219. The series of molecular | | | |
| | | signals initiated by binding of | | | |
| | | an extracellular ligand to a | | | |
| | | Notch receptor on the surface of | | | |
| | | the target cell. | | | |
| LEI_MYB_REGULATED_GENES | 325 | Myb-regulated genes | 8 | 0.0246 | 9.62E−06 |
| MORF_DDB1 | 246 | Neighborhood of DDB1 | 7 | 0.0285 | 1.40E−05 |

Epithelial Plasticity and Stem Cell-Like Behavior.

It is well appreciated that cells induced to undergo EMT activate stem cell pathways. Work presented here shows that AT3 cells that transitioned towards a more epithelial state, i.e. were involved in MET, also activated expression of stem cell-like markers. This finding suggested a broader relationship between plasticity and stem cell-like character or sternness, which was modeled using a Gibbs free energy diagram (FIG. 9). FIG. 9 shows a model comparing stem cell-like character and epithelial-mesenchymal phenotype. The x-axis represents the spectrum of epithelial to mesenchymal phenotypes and the y-axis represents the stem cell-like character of the cells. The left arrow represents an EMT and the right arrow represents an MET. The model posits that as cells transition back and forth along the epithelial and mesenchymal x-axis they course through states of varying sternness, and this property peaks at intermediate states between epithelial and mesenchymal phenotypes. The number of different states and the exact height of the barriers between states are speculative and are not meant to be taken as proportional. Two phenotypic transitions are shown, the first is a partial EMT (left arrow) and the second is a partial MET (right arrow). Both of these transitions result in states with higher stem cell-like character. It should be noted that the model also predicts that some EMTs, and equally some METs, will result in a decrease in sternness and indeed this has been observed when the highly aggressive human DKAT basal-type breast cancer cell line is induced to undergo EMT (N. D'Amato and V. Seewaldt, personal communication). The model also suggests a link between sternness, plasticity, and metastatic propensity, perhaps explained by activation of certain oncogenic pathways (e.g., PI3 kinase/Akt) and developmental pathways.

The model also predicts that cells with maximal stem-cell character, which by definition will be highly malignant, should display both epithelial and mesenchymal traits, because they inhabit intermediate states in the epithelialmesenchymal axis. The highly malignant rat adenocarcinoma AT3-T cells are in this type of state. Importantly, in humans with metastatic breast and prostate carcinomas many CTCs also exist in these intermediate states. These cells correlate with disease progression and are believed to be highly aggressive. A population of cells enriched in CTCs expressed RNAs encoding mesenchymal markers; however, the data did not indicate whether or not epithelial and mesenchymal markers were co-expressed in the same cell. Another clinical example of cells in intermediate states is found in sarcomatoid renal cell carcinomas, which have been shown to co-express epithelial markers, such as epithelial membrane antigen, and mesenchymal ones, like vimentin. These tumors, though rare (1-8% of renal tumors) are highly aggressive and difficult to treat. A similar situation may be found in carcinosarcomas of both the prostate and breast, highly aggressive, rare tumors with mixed epithelial and mesenchymal components but of clonal origin. It is not completely clear whether or not single cells in these tumor co-express epithelial and mesenchymal markers and are thus truly in intermediate states.

Finally, the model suggests that as sarcomas undergo MET they will activate stem cell-like pathways and become more aggressive. Indeed, there are many descriptions of sarcomas with mixed epithelial and mesenchymal components in close proximity as seen in some synovial- and osteo-sarcomas. New genetically-defined mouse models of soft tissue sarcoma should shed light on the existence and importance of cells intermediate cell states in progression of these tumors.

### Example 7

### Phenotypic Plasticity Among Human Circulating Tumor Cells

The experiments described above indicated that Dunning rat prostate adenocarcinoma cells that inhabit an intermediate phenotypic state are tumorogenic, metastatic, and possess stem cell-like antigens and cellular programs. To investigate whether or not similar transitional cells could play a role in human cancer, cancer cells isolated from blood of men with metastatic castrate resistant progressive prostate cancer (CRPC) or women with progressive metastatic breast cancer (mBC) were examined. Circulating tumor cells (CTCs) represent an ideal source of tissue to investigate evidence of this plasticity in vivo, given that these cells are likely to be in circulation prior to and during metastatic colonization. CTCs have both independent prognostic and predictive significance in multiple epithelial malignancies, including breast and prostate cancer. These cells can be collected, isolated, and analyzed for a variety of biomarkers relevant to cancer biology.

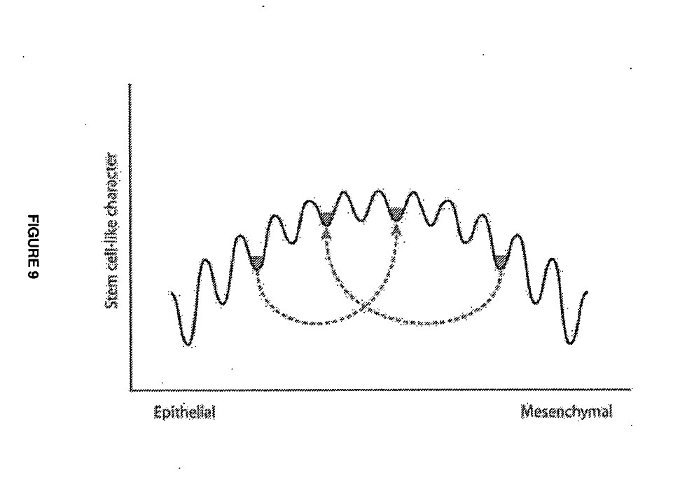

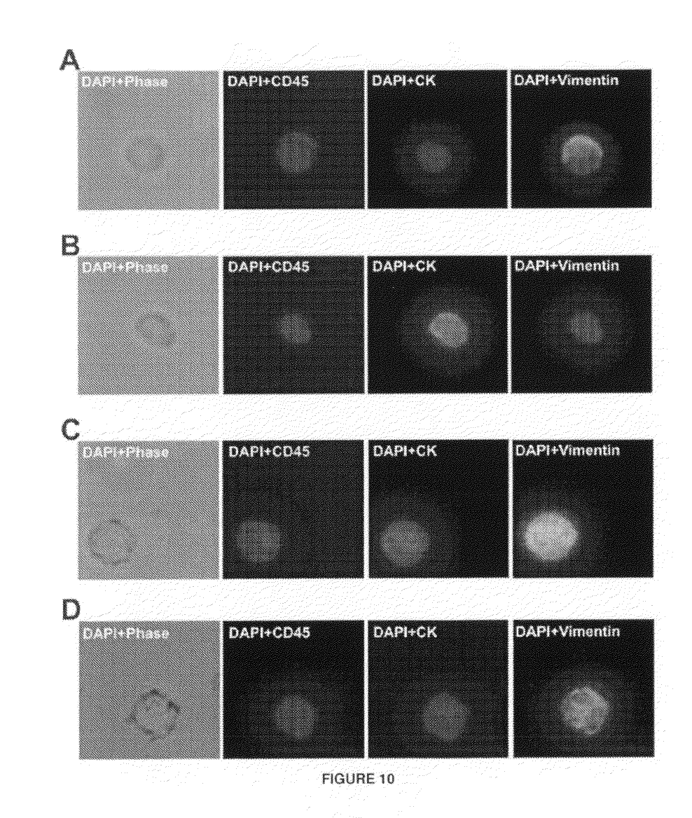

It was tested whether there was a high likelihood of finding transitional cells within a population of CTCs captured by FDA-approved EpCAM (Epithelial Cell Adhesion Molecule)-targeted ferromagnetic antibodies. These cells were interrogated for expression of CD45 (expressed in many leukocytes; FIG. 10A), cytokeratin (CK; an epithelial marker), and vimentin (a mesenchymal marker) by immunofluorescence. CTCs were defined as CD45-negative and CK-positive nucleated intact cells (FIG. 10B) and transitional CTCs were so defined if they additionally co-expressed vimentin (FIG. 10C-D). FIG. 10 shows that CTCs from patients with prostate adenocarcinoma stained positive for epithelial and mesenchymal markers. Triple staining was performed using anti-CD45 antibody labeled with Alexa 647, anti-cytokeratin (CK) antibody labeled with Alexa 555, and anti-vimentin antibody labeled with Alexa 488. Nuclei were labeled with DAPI. FIG. 10A shows an example of a leukocyte from a human peripheral blood mononuclear cell sample: CD45 (+), CK (−), and vimentin (+). Additionally, CD45 (+), CK (−), and vimentin (−) cells were observed. FIG. 10B shows an example of a CD45 (−), CK (+), and vimentin (−) cell from a patient with metastatic breast cancer. Such cells were counted as vimentin (−) CTCs in Table 6. FIG. 10C shows an example of a CD45 (−), CK (+), vimentin (+) from a patient with metastatic breast cancer. Such cells were counted as vimentin (+) CTCs in Table 6. FIG. 10D shows an example of a CD45 (−), CK (+), vimentin (+) from a patient with metastatic progressive castrate-resistant prostate cancer. Such cells were counted as vimentin (+) CTCs in Table 6.

Transitional CTCs co-expressed vimentin and CK in many of the patients with elevated CTC counts CTCs/7.5 mL by standard testing) (Table 6, FIG. 10). In fact, among nine patients with progressive metastatic CRPC and eight patients with progressive mBC, it was found that approximately 75% (range 0-100%, 85.5% in CRPC, 54% in mBC) of the CTCs stained for both CK and vimentin (FIG. 10C-D), indicating a transitional phenotype. These data indicated that circulating tumor cells in patients with metastatic breast and prostate cancer co-express epithelial (EpCAM and cytokeratin) and mesenchymal (vimentin) markers, and thus exist in a transitional phenotypic state, similar to that observed in our preclinical models.

TABLE 6
Circulating tumor cell (CTC) counts and vimentin
expression in patients with metastatic castration
resistant prostate or metastatic breast cancer.

| | | Ratio: |
|---|---|---|
| | CTC Count | vimentin (+) CTCs/ |
| Subject Number | (Cellsearch)* | Total CTC Count |
| Castrate-Resistance | | |
| Metastatic Prostates | | |
| Cancer | | |
| 1 | 5 | 4/6 |
| 2 | 41 | 11/11 |
| 3 | 45 | 6/10 |
| 4 | 626 | 5/8 |
| 5 | 110 | 17/21 |
| 6 | 182 | 5/6 |
| 7 | 17 | 13/16 |
| 8 | 19 | 33/34 |
| 9 | 34 | 12/12 |
| Total | | 106/124 (85.5%) |
| Metastatic Breast | | |
| Cancer | | |
| 1 | 21 | 0/6 |
| 2 | 7 | 2/2 |
| 3 | 8 | 4/4 |
| 4 | 21 | 1/2 |
| 5 | 12 | 2/2 |
| 6 | 188 | 21/22 |
| 7 | 138 | 8/20 |
| 8 | 377 | 6/23 |
| Total | | 44/81 (54.3%) |
| Overall Total | — | 150/205 (73.1%) |

*Column 2 represents the CTC count as determined by the standard Cellsearch EpCAM based method for each subject, while column 3 represents the number and proportion of CTCs counted manually that were found to express cytokeratin and co-express vimentin, expressed as a ratio and percentage.

Plasticity and CTCs.

The identification of plasticity among CTCs in a significant subset of patient samples offers several important clinical opportunities. Expression of plasticity may have prognostic or predictive value in patients with metastatic cancers, especially mBC where a significant range of values were shown for plasticity. Thus, the subset of patients with very high plasticity may have a more aggressive natural history and exhibit greater resistance to systemic treatments. In terms of diagnosis and utility as predictive biomarkers the data suggested that in addition to cells expressing both epithelial and mesenchymal markers there may be an unknown number of CTCs that have moved further towards the mesenchymal pole and are EpCAM negative. These cells will be missed by the FDA approved CellSearch® System and also by the Adna Test (AdnaGen AG) system and current microfluidic technologies, which enrich for CTCs by immunoabsorbtion of cells expressing MUC1 or EpCAM. Indeed, recent studies in breast cancer have suggested that “normal” type breast cancer cell lines that overexpress both EMT and stem cell antigens (CD44+, CD24−) may lack EpCAM and are thus not detectable by currently approved CTC detection systems. Therefore it is possible that the number of CTCs in patients with metastatic cancer is much higher than currently appreciated. Identification of this additional subset of CTC can provide greater prognostic value than CTC counts as currently determined, as well as earlier detection of CTCs and the metastatic potential in patients with earlier stage disease.

Furthermore, CTCs in intermediate states, which comprise the 50-75% of cells isolated herein from patients with metastatic breast and prostate cancer as well as those cells that may go undetected because they have undergone a more complete EMT, represent a therapeutic problem. It has been well documented that EMT alters drug sensitivity of lung cancer cells and it has been challenging to direct therapy to cancer cells with stem cell-like properties, perhaps because of their recalcitrance to undergo apoptosis.

While recent studies suggest both a screening method and actual compounds (e.g., salinomycin) that can selectively target cancer stem cells, these aggressive cells still represent a formidable therapeutic challenge. Thus, molecules comprising a binding agent that has binding specificity to an EMT biomarker described herein and linked to an anti-cancer agent provide additional therapeutic options.

### Example 8

### CTCs from Patients with Metastatic Breast and Prostate Cancer Express Vimentin and N-Cadherin

Eligible men had progressive metastatic CRPC (progression despite testosterone <50 ng/dL) and were about to begin a new systemic therapy. Eligible women had progressive metastatic breast cancer (mBC) and were about to begin a new systemic therapy. Baseline characteristics of patients (n=29) are presented in Table 7.

TABLE 7
Baseline characteristics of patients (n = 29)

| | |
|---|---|
| Metastatic Prostate | Metastatic Breast |
| (n = 17) | (n = 12) |

DEMOGRAPHICS

| | | | | |
|---|---|---|---|---|
| Age, median | 69 | (59-82) | 61.5 | (48-81) |

| | | |
|---|---|---|
| Race, Ethnicity | | |
| White, non-hispanic | 76% | 58% |
| Other, non-hispanic | 23% | 42% |

BASELINE DISEASE HISTORY

| Gleason Score, median | 7 | (7-9) | — | |
|---|---|---|---|---|
| ER/PR, % | — | | 75%/67% | |
| Baseline median PSA, | 396.4 | (14-13, 419.5) | — | |
| Range | | | | |
| Baseline Pain Score | 1 | (0-7) | 0 | (0-6) |
| (0-10), median | | | | |
| Karnofsky Performance | 90 | (70-100) | 90 (70-100) (n = 6) | |
| Status, median | | | | |
| # of Prior Hormonal | 2 | (0-5) | 2 | (0-4) |
| Therapies | | | | |
| Prior Chemotherapy | 47% | | 83% | |
| Baseline CTC | 40 | (4-828) | 13 | (0-1062) |
| Count, median | | | | |

METASTATIC SITES

| | | |
|---|---|---|
| Lymph Node | 65% | 50% |
| Liver | 24% | 50% |
| Lung | 47% | 42% |
| Bone | 94% | 75% |

CTCs were drawn into standard FDA-approved Cellsave tubes and processed within 48 hours using the CellSearch® methodology using EpCAM-based ferromagnetic capture. A CTC was defined as an intact nucleated (DAPI+) cell that expressed pan-CK and lacked expression of the leukocyte antigen CD45, and was enumerated using standard methods. A second Cellsearch® tube was collected and processed using EpCAM capture, and isolated cells were stained for CK (IgG1, AbD Serotec) labeled with Alexa 555, CD45 (IgG1, AbCam) labeled with Alexa 647, and either vimentin (IgG1, BD Biosciences) or N-Cadherin (IgG1, DAKO) using immunoflouresent labeling with Alexa 488. The proportion of CTCs staining positive for an EMT antigen was calculated from the total number of CTCs manually scored from the second tube. Positive controls using American Red Cross-derived PBMCs (CD45), PC3 prostate cancer cells (vimentin, N-cadherin), and T47D breast cancer cells (CK) were used for each marker. Negative controls using mock antibody were used to optimize the staining/scoring of each antigen.

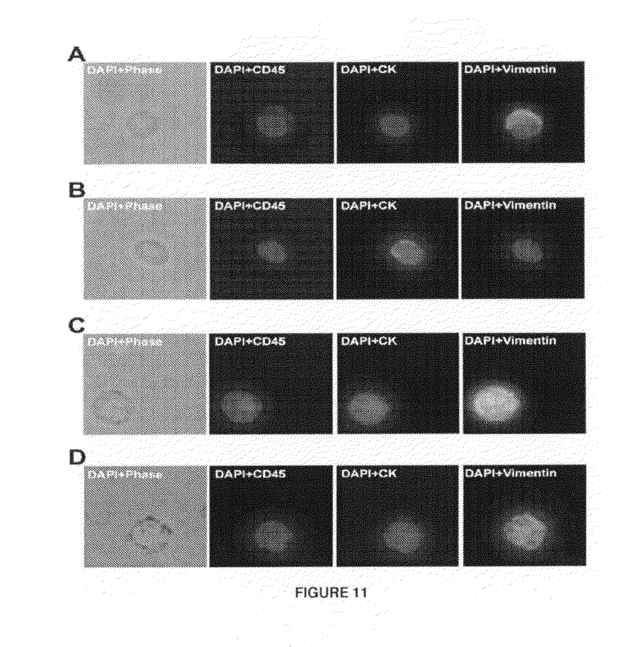

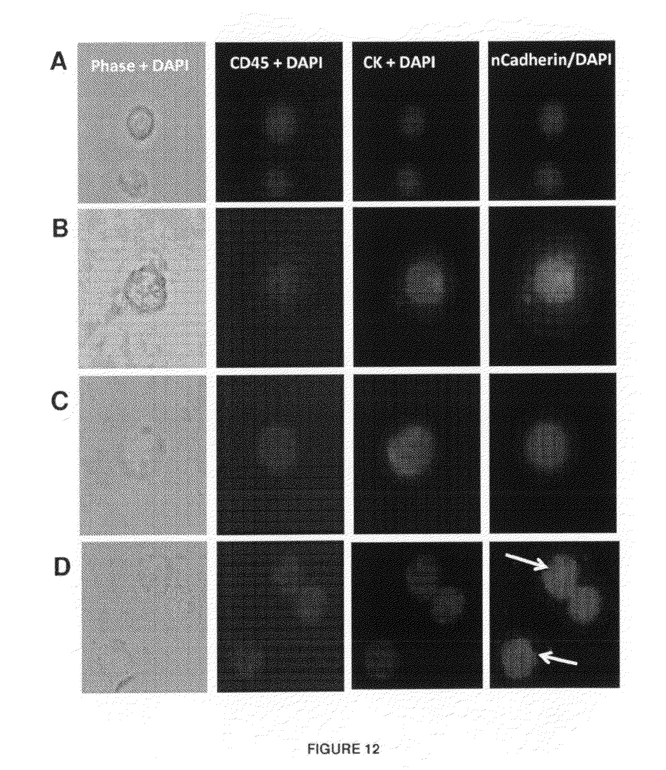

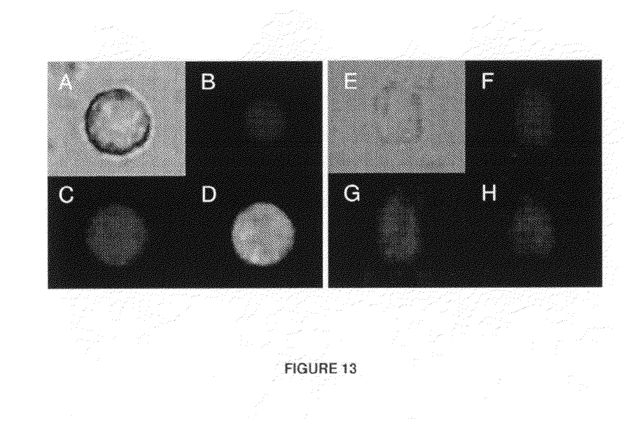

Prevalence of vimentin and CK co-expression in CTCs, and prevalence of N-cadherin and CK co-expression in CTCs are presented in Tables 8 and 9, respectively. Vimentin co-expression was detected in 17/20 (85%) patients with mCRPC or mBC and 78% of all CTCs. N-Cadherin co-expression was detected in 8/9 (89%) patients and 81% of CTCs. Immunofluorescent images of CTCs from patients with mCRPC and mBC are shown in FIG. 11 (A, a leukocyte; B, vimentin negative CTC (CRPC); C, vimentin positive CTC (BC); and D) vimentin positive CTC (CRPC)). Immunofluorescent images of CTCs from patients with mCRPC and mBC are shown in FIG. 12 (A, leukocyte; B, Ncad positive CTC (BC); C, Ncad negative CTC (BC); and D, two NCad positive CTCs (arrows) and I Ncad negative CTC (CRPC)). Immunofluorescent images of CTCs from patients with mCRPC and mBC are shown in FIG. 13 (A, Phase/DAPI; B, CD45/DAPI; C, CK/DAPI; D, Vimentin/DAPI positivity in a man with mCRPC; E, Phase/DAPI; F, CD45/DAPI; G, CK/DAPI; and H, Vimentin/DAPI negativity in a second man with mCRPC).

The data showed the co-expression of cytokeratin with the EMT antigens vimentin and N-cadherin in CTCs from men with metatastic CRPC and women with metastatic breast cancer. A majority of CTCs examined co-expressed CK and EMT proteins by immunofluorescent labeling. The majority of patients in this study had CTCs that co-expressed vimentin or N-cadherin suggesting potential epithelial plasticity during metastasis. The data suggests that CTCs can lack epithelial markers and provide methods for assessing patients with breast and prostate cancer as well as for the optimal detection of circulating tumor cells in other common malignancies.

TABLE 8

| | | | Ratio of: |
|---|---|---|---|
| | | CTC Count | Vimentin (+) CTCs/ |
| Subject Number | | (Cellsearch) | Total Manual CTC Count |
| castrate-resistant | 1 | 5 | 4/6 |
| metastatic prostate | 2 | 4 | 2/2 |
| cancer | 3 | 54 | 11/11 |
| | 4 | 45 | 6/10 |
| | 5 | 626 | 5/8 |
| | 6 | 110 | 17/21 |
| | 7 | 182 | 5/6 |
| | 8 | 17* | 13/16 |
| | 9 | 19 | 33/34 |
| | 10 | 34 | 12/12 |
| Total | | 1127 | 108/126 (86%) |
| metastatic breast | 1 | 13 | 0/6 |
| cancer | 2 | 85 | 2/2 |
| | 3 | 8 | 4/4 |
| | 4 | 21 | 1/2 |
| | 5 | 12 | 2/2 |
| | 6 | 188 | 21/22 |
| | 7 | 324** | 29/33 |
| | 8 | 377 | 6/23 |
| | 9 | 0 | 0/0 |
| | 10 | 3 | 0/3 |
| Total | | 884 | 65/97 (67%) |
| Overall Total | | — | 173/223 (78%) |

TABLE 9

| | | | Ratio of: |
|---|---|---|---|
| | | CTC Count | N-Cadherin (+) CTCs/ |
| Subject Number | | (Cellsearch) | Total Manual CTC Count |
| castrate-resistant | 1 | 45 | 13/19 |
| metastatic prostate | 2 | 12 | 5/7 |
| cancer | 3 | 10 | 8/8 |
| | 4 | 5 | 8/9 |
| | 5 | 12 | 4/4 |
| | 6 | 221 | 11/13 |
| | 7 | 828 | 81/96 |
| Total | | 1132 | 130/156 (83%) |
| metastatic breast | 1 | 1062 | 9/13 |
| cancer | 2 | 2 | 0/3 |
| Total | | 1064 | 9/16 (56%) |
| Overall Total | | — | 139/172 (81%) |

*Count from 3 months prior to baseline (no intervening therapy)
**Count from time point #2

In a second trial to test for the existence of transitional CTCs, blood was collected from 31 men with mCRPC and 16 women with mBC (see baseline characteristics for the patients in Table 10 and Table 11). CTCs were processed using the CellSearch® EpCAM-based immunocapture method and profiled for expression of CD45 (PTPRC) (a leukocyte marker), cytokeratins (CK) (epithelial markers), vimentin (VIM) and N-cadherin (CDH2) (mesenchymal markers), and CD133 (a stem cell marker) by immunofluorescence (IF) (Table 2). Leukocytes were defined as nucleated (DAPI positive), CD45-positive and CK-negative cells, whereas CTCs were defined as nucleated (DAPI positive), CD45-negative and CK-positive cells. Among CTCs we identified transitional cells as those that additionally expressed vimentin or N-cadherin.

TABLE 10
Baseline demographic and clinical characteristics
of the men with metastatic CPRC.

n = 31

DEMOGRAPHICS

| | | |
|---|---|---|
| Age, years (range) | 71 | (59-89) |

| | |
|---|---|
| Race, Ethnicity | |
| White, non-Hispanic | 71% |
| Black, non-Hispanic | 29% |

BASELINE DISEASE HISTORY

| | | |
|---|---|---|
| Median Gleason Score (range) | 8 | (5-10) |
| Median Baseline PSA ¹ (ng/dl, range) | 267.5 | (14.0-13,419.5) |
| Median Baseline Pain (range)² | 1 | (0-7) |
| Median Karnofsky Performance | 90 | (60-100) |
| Status (range) | | |
| Median Number of Prior Hormonal | 3 | (0-5) |
| Therapies (range) | | |

| | |
|---|---|
| Prior Chemotherapy | 65% |
| Prior Bisphosphonates | 71% |

SITES OF METASTATIC DISEASE

| | |
|---|---|
| Visceral (lung + liver) | 35% |
| Lymph Node Only | 0% |
| Bone metastatic: | |
| Bone Metastatic With Lymph Nodes | 39% |
| (no visceral metastases) | |
| Bone Metastatic Without Lymph Nodes | 26% |
| (no visceral metastases) | |

¹ PSA: prostate specific antigen.
²Pain is scored as a linear analog scale (0-10 range).

TABLE 11
Baseline characteristics of mBC patients.

n = 16

DEMOGRAPHICS

| | | |
|---|---|---|
| Median age (range) | 61 | (48-81) |

| | |
|---|---|
| Race, Ethnicity | |
| White, non-Hispanic | 44% |
| Black, non-Hispanic | 50% |
| Asian, non-hispanic | 6% |

BASELINE DISEASE HISTORY

| ER and/or PR positive disease | 56% | |
|---|---|---|
| HER2 positive disease (HER2 3+) | 0% | |
| Median Karnofsky Performance | 90 | (70-90) |
| Status (range) | | |
| Median Number of Prior EndocrineTherapies | 1 | (0-4) |
| (range) | | |
| Median Number of Prior Chemotherapies | 2 | (0-7) |

SITES OF METASTATIC DISEASE

| | |
|---|---|
| Visceral (lung or liver) | 75% |
| Lymph Node Only | 0% |
| Lymph Node, soft tissue, or contralateral | 13% |
| breast only | |
| Bone metastases only: | |
| Bone Metastatic With Lymph Nodes | 0% |
| (no visceral metastases) | |
| Bone Metastatic Without Lymph Nodes | 13% |
| (no visceral metastases) | |

Among ten men with mCRPC, CTCs co-expressed vimentin and CK in 10/10 (100%) patients, and by this criterion 108/126 (86%) of enumerated CTCs were transitional (Table 12, FIG. 14). Biopsies of bony metastases performed within one week of CTC collection in two of these patients revealed no vimentin expression in the CK positive tumor foci, but strong vimentin expression in the surrounding bone stroma, which lacks CK expression. These same patients had CTCs taken at the same time as the CT-guided tumor biopsy that commonly expressed co-expressed CK and vimentin. These findings are consistent with invasion and metastasis by transitional CTCs that subsequently undergo MET; alternatively, vimentin expression may be heterogeneously expressed in metastases, similar to CTC expression.

TABLE 12
Circulating tumor cell (CTC) and transitional
CTCs in patients with metastatic CRPC.

| | | Ratio: |
|---|---|---|
| Subject | CTC Count | Vimentin (+) CTCs/ |
| Number | (Cellsearch)ⁱ | Total Manual CTC Countⁱⁱ |
| 1 | 5 | 4/6 |
| 2 | 4 | 2/2 |
| 3 | 54 | 11/11 |
| 4 | 45 | 6/10 |
| 5 | 626 | 5/8 |
| 6 | 110 | 17/21 |
| 7 | 182 | 5/6 |
| 8 | 17 | 13/16 |
| 9 | 19 | 33/34 |
| 10 | 34 | 12/12 |
| Total | 1127 | 108/126 (86%) |
| | | Ratio: |
| Subject | CTC Count | N-Cadherin (+) CTCs/ |
| Number | (Cellsearch) | Total Manual CTC Count |
| 11 | 45 | 13/19 |
| 12 | 12 | 5/7 |
| 13 | 10 | 8/8 |
| 14 | 5 | 7/8 |
| 15 | 12 | 3/4 |
| 16 | 220 | 11/13 |
| 17 | 828 | 81/96 |
| 18 | 26 | 6/11 |
| 19 | 12 | 18/22 |
| 20 | 42 | 15/18 |
| Total | 1224 | 167/206 (81%) |
| | | Ratio: |
| Subject | CTC Count | CD133 (+) CTCs/ |
| Number | (Cellsearch) | Total Manual CTC Count |
| 21 | 485 | 38/38 |
| 22 | 16 | 6/11 |
| 23 | 91 | 15/21 |
| 24 | 6 | 0/0 |
| 25 | 36 | 29/29 |
| 26 | 27 | 9/9 |
| 27 | 43 | 10/15 |
| 28 | 2 | 0/0 |
| 29 | 23 | 12/14 |
| 30 | 38 | 23/26 |
| 31 | 30 | 12/17 |
| Total | 797 | 154/180 (86%) |

ⁱThe middle column represents the CTC Count from the FDA-approved Cellsearch ® enumeration of CTCs for each subject.
ⁱⁱRight column represents the ratio of vimentin (co-expression of vimentin ranged from 60-100% of cells in a given individual and did not correlate with CTC count (R² = 0.11)), N-cadherin (Co-expression of N-cadherin ranged from 55-100% of cells in a given individual, and did not correlate with CTC count (R² = −0.09)), or CD133 (CD133 co-expression ranged from 55-100% of evaluable cells in a given individual and did not correlate with CTC number (R² = 0.04)) expressing CTCs among the total number of CTCs that were manually enumerated. A CTC was defined as an intact DAPI positive (nucleated) cell that lacked CD45 expression and expressed cytokeratin.

TABLE 13
CTCs and transitional CTCs in patients with mBC.

| | | Ratio: |
|---|---|---|
| Subject | CTC Count | Vimentin (+) CTCs/ |
| Number | (Cellsearch)i | Total Manual CTC Countii |
| 1 | 21 | 0/6 |
| 2 | 7 | 2/2 |
| 3 | 8 | 4/4 |
| 4 | 21 | 1/2 |
| 5 | 12 | 2/2 |
| 6 | 188 | 21/22 |
| 7 | 324 | 29/33 |
| 8 | 377 | 6/23 |
| 9 | 0 | 0/0 |
| 10 | 3 | 0/3 |
| Total | 961 | 65/97 (67%) |
| | | Ratio: |
| Subject | CTC Count | N-Cadherin (+) CTCs/ |
| Number | (Cellsearch) | Total Manual CTC Count |
| 11 | 1062 | 9/13 |
| 12 | 2 | 0/3 |
| 13 | 147 | 52/59 |
| 14 | 6 | 2/5 |
| 15 | 33 | 15/15 |
| 16 | 2 | 0/0 |
| Total | 1252 | 78/95 (82%) |

Among the next cohort of 10 men with mCRPC, CTCs co-expressed N-cadherin and CK in 10/10 (100%) patients, and by this criterion 167/206 (81%) of CTCs were identified as transitional (Table 12, FIG. 15). Among 10 women with mBC, nine had detectable CTCs and of these, we found evidence of vimentin co-expression in seven (78%) patients, and 55/88 CTCs overall (63%) co-expressed vimentin (Table 13, FIG. 14). Among another six women with detectable CTCs and mBC, four had evidence of CK and N-cadherin co-expression, and overall 78/95 CTCs (82%) had N-cadherin expression, with significant heterogeneity in expression in a given individual (Table 13, FIG. 15). These data indicate that many CTCs in patients with mBC and mCRPC co-express epithelial (EpCAM and cytokeratin) and mesenchymal (vimentin, N-cadherin) markers, and thus exist in a transitional phenotypic state, similar to that observed in our preclinical models.

Given the expression of the stem cell associated antigen CD133 in transitional AT3-T cells, CD133 expression in CTCs from men with mCRPC was evaluated. CD133 was expressed in 11/11 (100%) men with CTCs, and in 154/180 (86%) of CTCs from these men (Table 12, FIG. 16). These data suggest that CTCs from patients with common epithelial malignancies inhabit transitional states characterized by co-expression of epithelial and mesenchymal markers as well as CD133, biomarkers that have been associated with stem-like properties, invasiveness, and chemoresistance.

| SEQUENCES | |
|---|---|
| SEQ ID NO: 1 | |
| N-cadherin (also known as cadherin-2, cdh2) | |
| From Mus musculus | |
| Gene No. 12558, Accession No. AB008811 | |
| nucleotide (mRNA), 4321 bp | |
| 1 | cacacacaca cgcacacaca cacacacaca cacttctcgg cgcgcacgac gcccgccctt |
| 61 | ctccccgccc cctccccagc tccttgatct cccgtctgtt ttattactcc tggtgcgagt |
| 121 | ccggcggact ccgaggcccg ctatttgtta ccaactcgct ctcattggcg gggaggagag |
| 181 | cagcggagaa gggggtgggg aggggagggg aagggaaggg gtggccactg ccggagccga |
| 241 | ctccgcgctg ctgttggtgc cgctgccgct tctgctgcct ctgctgccgc cgccgccgcc |
| 301 | tccggctcct cgctcggccc ctctccgcct ccatgtgccg gatagcggga gcgccgcgga |
| 361 | ccctgctgcc gcttctggcg gccttgcttc aggcgtctgt ggaggcttct ggtgaaattg |
| 421 | cattatgcaa gactggattt cctgaagatg tttacagcgc agtcttaccg aaggatgtgc |
| 481 | acgaaggaca gccccttctc aatgtgaaat tcagcaactg caatagaaaa aggaaagttc |
| 541 | agtatgaaag cagcgagcca gcagatttca aggtggacga ggacggcacg gtgtatgctg |
| 601 | tgagaagctt ccctctcact gcagagcagg caaagttcct gatatatgcc caagacaaag |
| 661 | aaacccagga aaagtggcag gtagctgtaa acctgagccg ggagccaacc ctgactgagg |
| 721 | agcctatgaa ggaaccacat gaaattgaag aaatagtatt ccctagacaa cttgccaagc |
| 781 | acagtggagc tctacaaagg cagaagagag actgggtcat cccgccaatc aacttgccag |
| 841 | aaaactccag aggacccttt cctcaagagc ttgtcagaat caggtctgat agagataaaa |
| 901 | acctttccct gagatacagc gtcactgggc caggagctga ccagcctcca acgggcatct |
| 961 | tcattatcaa ccccatctca ggacagctgt cagtcacaaa gcctctggat cgagagctga |
| 1021 | tagcccggtt tcacttgaga gcacatgcag tggacatcaa tggcaatcaa gtggagaacc |
| 1081 | ccattgacat tgtcatcaat gttattgaca tgaatgataa cagacctgag tttctgcacc |
| 1141 | aggtttggaa tgggtctgtt ccagagggat caaagcctgg gacgtatgtg atgacggtca |
| 1201 | ctgccattga tgcggatgat ccaaatgccc tgaatggaat gctgcggtac aggatcctgt |
| 1261 | cccaggcgcc cagcacacct tcacccaaca tgtttacaat caacaatgag actggggaca |
| 1321 | tcatcactgt ggcagctggt ctggatcgag agaaagtgca acagtatacg ttaataattc |
| 1381 | aagccacaga catggaaggc aatcccactt atggcctttc aaacacagcc acagccgtca |
| 1441 | tcacggtgac agatgtcaat gacaatcctc cagagtttac tgccatgact ttctacggag |
| 1501 | aagtccctga gaacagggtg gacgtcattg tagccaacct aactgtcacg gacaaagatc |
| 1561 | agccccacac gccggcctgg aatgcggcat acagaatcag tggtggagac cctacaggaa |
| 1621 | ggtttgccat cctgacagac cccaacagca atgatgggct agtcacagtg gtaaaaccaa |
| 1681 | ttgactttga aacgaatagg atgtttgtcc ttactgttgc tgcagaaaac caagtgccat |
| 1741 | tagctaaagg cattcagcac ccacctcagt cgacagccac tgtgtctgtg acagttattg |
| 1801 | atgtcaatga aaatccttat tttgccccaa atcctaaaat cattcgccaa gaggaaggcc |
| 1861 | tccacgcagg taccatgctg accacgctca ctgctcagga ccccgatcga tatatgcaac |
| 1921 | agaatatcag atacacaaaa ttgtctgatc ctgccaactg gctgaaaata gaccccgtga |
| 1981 | atgggcagat cactactatt gccgttttgg acagagaatc gccaaatgta aaaaacaaca |
| 2041 | tctataatgc taccttcctt gcttctgaca atggaatccc gcctatgagt gggacaggaa |
| 2101 | cactgcaaat ctatttactt gatatcaatg acaacgcccc tcaggtgtta cctcaagagg |
| 2161 | cggagacctg tgaaactcca gaacccaact caattaacat cacagcactt gattatgaca |
| 2221 | tagacccaaa cgccgggccg ttcgcgtttg atcttccctt atctccagtg actattaaaa |
| 2281 | gaaactggac catcaaccgg cttaatggtg attttgctca gctcaattta aagataaaat |
| 2341 | ttttggaagc tggtatctat gaagttccca tcattatcac agattcaggg aatcccccca |
| 2401 | agtccaacat ttccatcctg cgtgtgaaag tttgtcagtg tgactccaat ggagactgca |
| 2461 | cggacgtgga caggatcgtg ggtgcagggc ttggcacggg cgccatcatc gctatccttc |
| 2521 | tgtgtatcat catcctgctg atccttgttc tcatgtttgt ggtatggatg aaacggcggg |
| 2581 | ataaagagcg ccaagccaag cagcttttaa ttgacccaga agatgatgta agagataata |
| 2641 | tattgaaata tgatgaagaa ggtggaggag aagaagacca ggactatgac ttgagccagc |
| 2701 | tccagcaacc agatactgtg gagcctgatg ccatcaagcc cgtgggaatc agacggctag |
| 2761 | acgagaggcc tatccatgct gagccacagt acccagtccg atccgcagcc ccacaccctg |
| 2821 | gggatattgg ggacttcatt aatgagggcc ttaaagctgc tgacaacgac cccacggcgc |
| 2881 | caccgtatga ctccctctta gtctttgact acgagggcag cggctccacg gctggctcct |
| 2941 | tgagctccct caactcctcc agtagcggtg gggaccagga ctatgactac ctgaatgact |
| 3001 | ggggaccccg cttcaagaaa ctggcggaca tgtacggcgg tggtgacgac tgaacggcag |
| 3061 | gacggacttg gcttttggac aagtatgaac agtttcacct gatattccca aaaaaaagca |
| 3121 | tacagaagct aggctttaac tctgtagtcc actagcaccg tgcttgctgg aggctttggc |
| 3181 | gtaggctgcg aaccagtttg ggctcccagg gaatatcagt gatccaatac tgtctggaaa |
| 3241 | acaccgagct cagctacact tgaattttac agtaaagaag cactgggatt tatgtgcctt |
| 3301 | tttgtacctt tttcagattg gaattagttt tctgtttaag gctttaatgg tactgatttc |
| 3361 | tgaaatgata aggaaaagac aaaatatttt gtggcgggag cagaaagtta aatgtgatac |
| 3421 | gcttcaaccc acttttgtta caatgcattt gcttttgtta agatacagaa cgaaacaacc |
| 3481 | agattaaaaa aaattaactc atggagtgat tttgttacct ttggggtggg ggggatgaga |
| 3541 | ccacaagata ggaaaatgta cattacttct agttttagac tttagatttt tttttttcac |
| 3601 | taaaatctta aaacttacgc agctggttgc agataaaggg agttttcata tcaccaattt |
| 3661 | gtagcaaaat gaattttttc ataaactaga atgttagaca cattttggtc ttaatccatg |
| 3721 | tacacttttt tattttctgt attttttcca cctcgctgta aaaatggtgt gtgtacataa |
| 3781 | tgtttatcag catagactat ggaggagtgc agagaactcg gaacatgtgt atgtattatt |
| 3841 | tggactttgg attcaggttt tttgcatgtt aatatctttc gttatgggta aagtatttac |
| 3901 | aaaacaaagt gacatttgat tcaactgttg agctgtagtt agaatactca atttttaatt |
| 3961 | ttttaatttt ttttaaattt ttttattttc tttttgtttg tttcgttttg gggaggggta |
| 4021 | aaagttctta gcacaatgtt ttacataatt tgtaccaaaa aaattacaca caaaaaaaaa |
| 4081 | aaaaagaaaa gaaaagaaaa gtgaaagggg tggcctgttt cttgcagcac tagcaagtgt |
| 4141 | gtgtttttaa aaaacaaaac aaacaaacaa aaaaataaat aaaaagagga aaaagaaaaa |
| 4201 | aaaaaaagct tttaaactgg agagacttct gaaacagctt tgcgtctgtg ttgtgtacca |
| 4261 | gaatacaaac aatacacctc tgaccccagc gttctgaata aaaagctaat tttggatctg |
| 4321 | g |
| SEQ ID NO: 2 | |
| N-cadherin (also known as cadherin-2, cdh2) | |
| From Mus musculus | |
| Gene No. 12558, Accession No. AB008811 | |
| polypeptide, translation of SEQ ID NO: 1 | |
| MCRIAGAPRTLLPLLAALLQASVEASGEIALCKTGFPEDVYSAV | |
| LPKDVHEGQPLLNVKFSNCNRKRKVQYESSEPADFKVDEDGTVYAVRSFPLTAEQAKF | |
| LIYAQDKETQEKWQVAVNLSREPTLTEEPMKEPHEIEEIVFPRQLAKHSGALQRQKRD | |
| WVIPPINLPENSRGPFPQELVRIRSDRDKNLSLRYSVTGPGADQPPTGIFIINPISGQ | |
| LSVTKPLDRELIARFHLRAHAVDINGNQVENPIDIVINVIDMNDNRPEFLHQVWNGSV | |
| PEGSKPGTYVMTVTAIDADDPNALNGMLRYRILSQAPSTPSPNMFTINNETGDIITVA | |
| AGLDREKVQQYTLIIQATDMEGNPTYGLSNTATAVITVTDVNDNPPEFTAMTFYGEVP | |
| ENRVDVIVANLTVTDKDQPHTPAWNAAYRISGGDPTGRFAILTDPNSNDGLVTVVKPI | |
| DFETNRMFVLTVAAENQVPLAKGIQHPPQSTATVSVTVIDVNENPYFAPNPKIIRQEE | |
| GLHAGTMLTTLTAQDPDRYMQQNIRYTKLSDPANWLKIDPVNGQITTIAVLDRESPNV | |
| KNNIYNATFLASDNGIPPMSGTGTLQIYLLDINDNAPQVLPQEAETCETPEPNSINIT | |
| ALDYDIDPNAGPFAFDLPLSPVTIKRNWTINRLNGDFAQLNLKIKFLEAGIYEVPIII | |
| TDSGNPPKSNISILRVKVCQCDSNGDCTDVDRIVGAGLGTGAIIAILLCIIILLILVL | |
| MFVVWMKRRDKERQAKQLLIDPEDDVRDNILKYDEEGGGEEDQDYDLSQLQQPDTVEP | |
| DAIKPVGIRRLDERPIHAEPQYPVRSAAPHPGDIGDFINEGLKAADNDPTAPPYDSLL | |
| VFDYEGSGSTAGSLSSLNSSSSGGDQDYDYLNDWGPRFKKLADMYGGGDD | |
| SEQ ID NO: 3 | |
| O-cadherin (also known as cadherin-11, cdh 11, or ob-cadherin) | |
| From Xenopus laevis | |
| Gene No. 100337621, Accession No. AF002983 | |
| nucleotide (RNA), 3237 bp | |
| 1 | tcggcacgag ctggagtgta caggactttt aagatgctgc tgggtgtctg cactgtgtcc |
| 61 | atgtgaatgt ggcattttta ttttgaattc cctccggaga caagatttca tcaagagttt |
| 121 | cctttggata ttaagtcaaa gtgcaagcaa tggagattct ctataagaag gcaataatct |
| 181 | gggggattta ctaaaattaa acaaacagat tgacattcgc tggatttatc aagcaatttt |
| 241 | gcatttacaa cactaccaaa aatgaagaaa gacttttgct tacacggttt acttttatgt |
| 301 | ttgggaattg cgtattgtag tcatgccaca tctttaagaa aaaacaataa actaaggcaa |
| 361 | tcattccatg gtcaccatga aaaaggcaaa gaagggcaag ttttacatag gtcaaagaga |
| 421 | ggatgggttt ggaatcaatt ttttgtaata gaagaataca ccggaccaga tcctgtactc |
| 481 | gttggacggc ttcactcaga tgttgactct ggagattgga agataaaata catactctca |
| 541 | ggagagggtg ctgggaccat ttttgtcatt gatgacaaat cagggaatat ccatgcaacc |
| 601 | aagaccctgg atcgagaaga aagggctcag tataccttaa tggctcaggc agttgacaga |
| 661 | gaaacaaata aaccactgga accaccatca gagtttatcg ttaaagttca agacataaat |
| 721 | gataatcccc cggagttctt gcatgaaaac taccacgcaa atgtgcctga gatgtccaat |
| 781 | gtgggtacat cagtaattca agtaacagcc tctgatgcag atgatccaac atatggaaac |
| 841 | agcgctaagc ttgtgtatag tattctcgaa gggcagccat atttttcagt cgaagcacaa |
| 901 | tcaggaatca ttaggactgc ccttccaaac atggacagag aagccaagga agaataccat |
| 961 | gttgttattc aagcaaagga tatgggagga catatgggag gactctcagg gacaactaaa |
| 1021 | gtgacaataa cgctgacaga tgtcaatgac aatccaccaa agtttccaca aagtgcgtac |
| 1081 | cccatgtctg tgtcagaagc tgctgtccca ggggaagagg ttggcagaat aaaagctaaa |
| 1141 | gatccagaca ttggagaaaa tggcttaata aagtaccgta ttcttgaagg agatggggca |
| 1201 | gagatgtttg aaatcacagc tgattatgta actcaggaag gcgttgtaaa gctaaaaaag |
| 1261 | gtggtggatt atgaaaccaa gaagttctac agtatgaagg ttgaagctgt caacgttcat |
| 1321 | attgatccca gattccttag ccggggacca ttcaaagaca ctgctactgt taagatctca |
| 1381 | gtagaggatt ttgatgaacc gcctattttc ttagaaagaa gttacatttt ggaagtatat |
| 1441 | gaaaatgctc catcggatac tgtggtcgga agagtgcacg ctaaagaccc agatgctgct |
| 1501 | aacagcccaa ttaggtattc aatcgatcgc cacactgacc ttgacagatt cttcagcatc |
| 1561 | aacccagagg atggtgtcat caaaaccaca aagggtttgg atagagagga aagcccttgg |
| 1621 | cacaacatct cagtcattgc aactgaagtc cacaatcgaa ttcatgaaac tagagttcca |
| 1681 | gtagctatta aagtcttgga taagaatgac aatgctccgg aatttgcaaa gccctatgaa |
| 1741 | gcttttgtct gtgaaaatgc tccaatcaat caggagtttt tgaccatcac tgcagtagat |
| 1801 | aaagatgata cagccaatgg acttcgtttt ctctttagtt tccccccaga aattgtacat |
| 1861 | ccaaatccaa atttcaccat aatagacaaa cgagataaca cagcaagcat ccgtgttggc |
| 1921 | cgtggagttt tcagccgaca gaaacaagac ttgtatttgg ttcctattgt tataagtgat |
| 1981 | gggggaagcc caccgatgag cagcaccaat accctttctg tccgaatctg cagttgcaat |
| 2041 | agtgatggat cccaactatc ttgtaatgct gaaccccaat cccttaacgc tggactcagt |
| 2101 | actggagcac tgattgcaat ccttgcttgc attgtaattt tattagtgat tgtggttttg |
| 2161 | tttgtgactc tgaggagaga gaagaaggaa cctctaattg tctttgaaga ggaagatatc |
| 2221 | cgggaaaata taattacata tgatgatgaa ggtggtggag aggaagacac cgaagcattt |
| 2281 | gacattgcaa cactgcagaa tcctgatggg attaatggat ttatgccacg gaaagatatc |
| 2341 | aaacccgaat ttcaatataa ccccagagat attggaataa gaccagcacc aaacagtgtt |
| 2401 | gacgttgatg acttcattaa cacaaggata catgaggccg ataatgaccc tgcagctccg |
| 2461 | ccttatgact ccattcagat ctatggatac gaagggagag gttctgtggc tggctctctt |
| 2521 | agttcattag agtcagcctc tacagattca gatttggact atgattatct acaaaactgg |
| 2581 | ggacctcgat ttaagaaact agcaaattta tatgggtcca aagacacttg tgaagatgat |
| 2641 | tcttaacaaa taagttctga atttggcctt atgaactgca taatgtactg aaatatccag |
| 2701 | agtaaacatt aacaggtatt tttttaaagg aaaacatgaa aaaggcttct ttaaccttcc |
| 2761 | aaggtttaca aacaggattc cttccaaaac aagaactgtt aaatggtggt ggatactgtg |
| 2821 | aaaaccctat ggcctgtgta gaagttgtgt attcattttt ttttttgttt tttgtttttt |
| 2881 | ttccaagaaa ccacttgtaa aatgcagcct atttaaggga atggaaatgc aggaaaaacg |
| 2941 | caacaaaaaa ggggaatctt tacagtatta aacataacca tcaaatcttc tcaaacaaag |
| 3001 | cttccacaca aaaaaaaaaa aagataacag ttttgagctg taatttcgcc ttaaactatg |
| 3061 | gacactttat atgtagtgca tttttaaact tgaaaaaaat atatatataa tatccagcca |
| 3121 | gcttcaatcc atataatgta tgtacagtaa aatgtacaat tattctgtct cttgagcatc |
| 3181 | agacttgtta ctgctgattc ttgtaaatct tttttgctta taatcccctc gtgccga |
| SEQ ID NO: 4 | |
| O-cadherin (also known as cadherin-11, cdh11, or ob-cadherin) | |
| From Xenopus laevis | |
| Gene No. 100337621, Accession No. AF002983 | |
| polypeptide, translation of SEQ ID NO: 3 | |
| MKKDFCLHGLLLCLGIAYCSHATSLRKNNKLRQSFHGHHEKGKE | |
| GQVLHRSKRGWVWNQFFVIEEYTGPDPVLVGRLHSDVDSGDWKIKYILSGEGAGTIFV | |
| IDDKSGNIHATKTLDREERAQYTLMAQAVDRETNKPLEPPSEFIVKVQDINDNPPEFL | |
| HENYHANVPEMSNVGTSVIQVTASDADDPTYGNSAKLVYSILEGQPYFSVEAQSGIIR | |
| TALPNMDREAKEEYHVVIQAKDMGGHMGGLSGTTKVTITLTDVNDNPPKFPQSAYPMS | |
| VSEAAVPGEEVGRIKAKDPDIGENGLIKYRILEGDGAEMFEITADYVTQEGVVKLKKV | |
| VDYETKKFYSMKVEAVNVHIDPRFLSRGPFKDTATVKISVEDFDEPPIFLERSYILEV | |
| YENAPSDTVVGRVHAKDPDAANSPIRYSIDRHTDLDRFFSINPEDGVIKTTKGLDREE | |
| SPWHNISVIATEVHNRIHETRVPVAIKVLDKNDNAPEFAKPYEAFVCENAPINQEFLT | |
| ITAVDKDDTANGLRFLFSFPPEIVHPNPNFTIIDKRDNTASIRVGRGVFSRQKQDLYL | |
| VPIVISDGGSPPMSSTNTLSVRICSCNSDGSQLSCNAEPQSLNAGLSTGALIAILACI | |
| VILLVIVVLFVTLRREKKEPLIVFEEEDIRENIITYDDEGGGEEDTEAFDIATLQNPD | |
| GINGFMPRKDIKPEFQYNPRDIGIRPAPNSVDVDDFINTRIHEADNDPAAPPYDSIQI | |
| YGYEGRGSVAGSLSSLESASTDSDLDYDYLQNWGPRFKKLANLYGSKDTCEDDS | |
| SEQ ID NO: 5 | |
| CD133 (also known as PROM-1, prominin-1), isoform 2 | |
| From Mus musculus | |
| Gene No. 19126, Accession No. BC028286 | |
| nucleotide (mRNA), 3701 bp | |
| 1 | gtccaatcag tgcgctcaga ctcagagccc taggctcctg ctctttaaat taccgagcct |
| 61 | tgtggagacc ccggcacctg gccttaagct cagccctgag gatggtactt tgagtgaatg |
| 121 | accaccttgg agaccgttct tctgtttccc ttgttaccag ccaggaggca gaagagtcca |
| 181 | ccggtccagg aaagacccat ttcccttgag tttccagaaa gtacctcatg cttgagagat |
| 241 | caggccaaca actatggctc tcgtcttcag tgccctgctg ttactggggc tgtgtggaaa |
| 301 | gatctcttca gaaggtcagc ctgcattcca taacactcct ggggctatga attatgaatt |
| 361 | gcctaccacc aaatatgaga cccaagatac cttcaatgct gggattgttg gccctctcta |
| 421 | caaaatggtg cacatcttcc tcaacgtggt ccagccgaat gacttccctc tagatttgat |
| 481 | caaaaaactc atacagaaca agaactttga catctcagtt gattccaagg agccagaaat |
| 541 | catagtcttg gctctgaaga ttgccctcta tgagatcgga gtccttatct gcgccatcct |
| 601 | gggactgctg ttcattatcc tcatgcctct ggtgggctgc ttcttttgta tgtgccgttg |
| 661 | ctgcaacaaa tgcggcggag agatgcacca gcggcagaag cagaatgcgc catgcaggag |
| 721 | gaagtgcttg ggcctctccc tcctggtgat ttgtctgctc atgagccttg gcattatata |
| 781 | tggctttgtg gctaaccagc agaccaggac tcggatcaaa gggacccaga aactggcaaa |
| 841 | gagcaatttc agagactttc aaacactcct gactgaaaca ccaaagcaaa ttgactatgt |
| 901 | agtggagcag tacaccaaca ccaagaacaa ggcattctca gacctggatg gcatcggctc |
| 961 | cgtgctggga ggcagaataa aggaccaact aaaacccaaa gtaactcctg tcctcgaaga |
| 1021 | gattaaggcc atggcgacag ccatcaaaca gaccaaggat gccctgcaga acatgagcag |
| 1081 | cagcctgaaa agtctccaag atgcagccac ccagctcaat accaacctga gctctgtgag |
| 1141 | aaacagcatc gagaattcgc tcagcagcag tgactgtacc tcagatccag ccagcaagat |
| 1201 | ctgcgatagc atcagaccaa gcctaagcag tctggggagc agcctcaatt caagtcagct |
| 1261 | cccatcagtg gatagagaac tcaacactgt tactgaagtc gacaaaactg atctggagag |
| 1321 | cctcgtcaaa agggggtata cgacaattga tgaaataccc aatacaatac aaaaccaaac |
| 1381 | tgtggatgtc atcaaagacg tcaaaaatac cttggactcc attagctcca acattaagga |
| 1441 | catgagccaa agtattccta ttgaggatat gctgttacag gtctcccatt accttaataa |
| 1501 | cagcaacaga tacttaaacc aggagctgcc caagctggaa gaatatgact cgtactggtg |
| 1561 | gctgggtggc ttgattgtct gctttctgct gactctcatt gtgaccttct ttttcctggg |
| 1621 | cttgctgtgt ggtgtgtttg gctatgacaa gcatgccacc ccaactagaa gaggctgtgt |
| 1681 | gtccaacact ggaggcatct tcctcatggc tggggttgga ttcggcttcc ttttttgctg |
| 1741 | gatattgatg atccttgtgg ttcttacgtt tgttgttggt gcaaatgtgg aaaagttgct |
| 1801 | ctgcgaacct tatgaaaaca agaaattatt acaggttttg gacactccct atctgctcaa |
| 1861 | ggaacaatgg caattttatc tttctggcat gctattcaat aacccagaca ttaacatgac |
| 1921 | ctttgagcaa gtctacaggg attgcaaaag aggtcgaggt atatatgctg cttttcagct |
| 1981 | tgagaatgtc gtcaacgtca gtgatcattt caacattgac cagatttctg aaaacataaa |
| 2041 | tacggagttg gaaaacctga atgtgaacat tgatagcatt gaactgttgg ataacacagg |
| 2101 | aaggaagagc ctcgaggact ttgcacattc tgggatagat acaatcgatt attccacata |
| 2161 | cttgaaggag actgagaaat cccctactga agtgaatctg ctgacatttg cctctaccct |
| 2221 | ggaagcaaaa gcaaaccagt tgcctgaagg aaagctgaaa caggccttct tactggatgt |
| 2281 | acagaatata agagccatcc accagcatct cctccctcct gtgcagcaat cactgaaatt |
| 2341 | tgtgagggtg aggaatacgt taagacaaag tgtctggacc ctccagcaaa caagcaacaa |
| 2401 | gttgccggag aaagtgaaga agatccttgc ctctttggac tctgttcagc atttcctcac |
| 2461 | caataacgtt tccctcatcg ttatcgggga aacgaagaag tttgggaaaa caatactagg |
| 2521 | ctactttgaa cattatctgc actgggtctt ttatgccatc acagagaaga tgacatcctg |
| 2581 | caaacccatg gccaccgcga tggactctgc tgttaatggc attctgtgtg gctatgttgc |
| 2641 | ggaccctctg aatttgttct ggttcggcat agggaaagcc acggtgctct tacttccggc |
| 2701 | tgtaatcatt gctatcaagc tggccaagta ctatcgcagg atggattcag aggatgtata |
| 2761 | cgacgacccg tctcgatact gacaactgga gttgaagctg cttgaacaac aagatagtca |
| 2821 | acatggaaag catcacagat tttggatagt ttctgagtct tctagaacgt tccaagtgca |
| 2881 | gaagaaacct ggtggagact caggcgggca ctaggaacat ggcatcagtg gtcttagggt |
| 2941 | agcactttgt caggaatgaa cagtcatcat ggttataatc cacatatcca ttgcaactca |
| 3001 | tgaatgattc tctcctgttt tgtttttaac ttttcttttt acactgattt tctatttaga |
| 3061 | cactaaaaca tataggggtg cttattcccc ctggatacat ttacctgtga accagctatt |
| 3121 | ccggtgtcat agctgggtac ctaacttact tccatatgtg aagtgtgcta aacacaaacc |
| 3181 | agtttacaga agagatgtat tttgtgtata gtaaactgta tatataccct tttaccacag |
| 3241 | tcagtttttt aaacaaatga atactctaga tttttcttct aaatgaggtt actgttgggg |
| 3301 | tggttgtgac ctagtgatgc tgtagaaagg agtctgcatt cactaaaagt gtgtcaacct |
| 3361 | agagcaggca atgcccttcc ttgtggattt ctgtctgctc gttttggagc tacctgcggt |
| 3421 | ttagaaatag aattcaagaa caatcacgga gtttcccact tgatgccact gccaaagtca |
| 3481 | gaacaaggga tcttgagaga aggaactgtc gctcagctgg gagcggaatc attatcgcaa |
| 3541 | tcacaggtcc tggttcacag tttagtggca ctctctggtt tgtaagaatg ggcattacgt |
| 3601 | tcagtgtcat ctggtcatct gtgatgtgtg tcatcagcct gtcctgatgt tgagatttaa |
| 3661 | aataaagcat gaatgaacag aaaaaaaaaa aaaaaaaaaa a |
| SEQ ID NO: 6 | |
| CD133 (also known as PROM-1, prominin-1), isoform 2 | |
| From Mus musculus | |
| Gene No. 19126, Accession No. BC028286 | |
| polypeptide, translation of SEQ ID NO: 5 | |
| MALVFSALLLLGLCGKISSEGQPAFHNTPGAMNYELPTTKYETQ | |
| DTFNAGIVGPLYKMVHIFLNVVQPNDFPLDLIKKLIQNKNFDISVDSKEPEIIVLALK | |
| IALYEIGVLICAILGLLFIILMPLVGCFFCMCRCCNKCGGEMHQRQKQNAPCRRKCLG | |
| LSLLVICLLMSLGIIYGFVANQQTRTRIKGTQKLAKSNFRDFQTLLTETPKQIDYVVE | |
| QYTNTKNKAFSDLDGIGSVLGGRIKDQLKPKVTPVLEEIKAMATAIKQTKDALQNMSS | |
| SLKSLQDAATQLNTNLSSVRNSIENSLSSSDCTSDPASKICDSIRPSLSSLGSSLNSS | |
| QLPSVDRELNTVTEVDKTDLESLVKRGYTTIDEIPNTIQNQTVDVIKDVKNTLDSISS | |
| NIKDMSQSIPIEDMLLQVSHYLNNSNRYLNQELPKLEEYDSYWWLGGLIVCFLLTLIV | |
| TFFFLGLLCGVFGYDKHATPTRRGCVSNTGGIFLMAGVGFGFLFCWILMILVVLTFVV | |
| GANVEKLLCEPYENKKLLQVLDTPYLLKEQWQFYLSGMLFNNPDINMTFEQVYRDCKR | |
| GRGIYAAFQLENVVNVSDHFNIDQISENINTELENLNVNIDSIELLDNTGRKSLEDFA | |
| HSGIDTIDYSTYLKETEKSPTEVNLLTFASTLEAKANQLPEGKLKQAFLLDVQNIRAI | |
| HQHLLPPVQQSLKFVRVRNTLRQSVWTLQQTSNKLPEKVKKILASLDSVQHFLTNNVS | |
| LIVIGETKKFGKTILGYFEHYLHWVFYAITEKMTSCKPMATAMDSAVNGILCGYVADP | |
| LNLFWFGIGKATVLLLPAVIIAIKLAKYYRRMDSEDVYDDPSRY | |
| SEQ ID NO: 7 | |
| FGFR2 IIIc | |
| From Mus musculus | |
| Gene No. 14183, Accession No. M86441 | |
| nucleotide (mRNA), 3306 bp | |
| 1 | gaattcccgc gcggccgcca gagctccggc ccgggggctg cctgtgtgtt cctggcccgg |
| 61 | cgtggcgact gctctccggg ctggcggggg ccgggcgtga gcccgggcct cagcgttcct |
| 121 | gagcgctgcg agtgttcact actcgccagc aaagtttgga gtaggcaacg caagctccag |
| 181 | tcctttcttc tgctgctgcc cagatccgag agcagctccg gtgtatgtct agctgttctg |
| 241 | cgatcccggc gcgcgtgaag cctcggaacc ttggcgccgg ctgctaccca aggaatcgtt |
| 301 | ctctttttgg agttttcctc cgagatcatc gcctgctcca tcccgatcca ctctgggctc |
| 361 | cggcgcagca ccgagcgcag aggagcgctg ccattcaagt ggcagccaca gcagcagcag |
| 421 | cagcagcagt gggagcagga acagcagtaa caacagcaac agcagcacag ccgcctcaga |
| 481 | gctttgctcc tgagcccctg tgggctgaag gcattgcagg tagcccatgg tctcagaaga |
| 541 | agtgtgcaga tgggattacc gtccacgtgg agatatggaa gaggaccagg gattggcact |
| 601 | gtgaccatgg tcagctgggg gcgcttcatc tgcctggtct tggtcaccat ggcaaccttg |
| 661 | tccctggccc ggccctcctt cagtttagtt gaggatacca ctttagaacc agaagagcca |
| 721 | ccaaccaaat accaaatctc ccaaccagaa gcgtacgtgg ttgcccccgg ggaatcgcta |
| 781 | gagttgcagt gcatgttgaa agatgccgcc gtgatcagtt ggactaagga tggggtgcac |
| 841 | ttggggccca acaataggac agtgcttatt ggggagtatc tccagataaa aggtgccaca |
| 901 | cctagagact ccggcctcta tgcttgtact gcagctagga cggtagacag tgaaacttgg |
| 961 | atcttcatgg tgaatgtcac agatgccatc tcatctggag atgatgagga cgacacagat |
| 1021 | agctccgaag acgttgtcag tgagaacagg agcaaccaga gagcaccgta ctggaccaac |
| 1081 | accgagaaga tggagaagcg gctccacgct tgtcctgccg ccaacactgt gaagttccgc |
| 1141 | tgtccggctg gggggaatcc aacgtccaca atgaggtggt taaaaaacgg gaaggagttt |
| 1201 | aagcaggagc atcgcattgg aggctataag gtacgaaacc agcactggag ccttattatg |
| 1261 | gaaagtgtgg tcccgtcaga caaaggcaac tacacctgcc tggtggagaa tgaatacggg |
| 1321 | tccatcaacc acacctacca cctggatgtc gttgaacgtt caccacaccg tcccatcctc |
| 1381 | caagctggac tgcctgcaaa tgcctccacg gtggtcggag gggatgtgga gtttgtctgc |
| 1441 | aaggtttaca gcgatgccca gccccacatc cagtggatca agcacgtgga aaagaacggc |
| 1501 | agtaaaaacg ggcctgatgg gctgccctac ctcaaggttc tgaaagctgc cggtgttaac |
| 1561 | accacggaca aagagattga ggttctctat attcggaatg taacttttga ggatgctggg |
| 1621 | gaatatacgt gcttggcggg taattctatc gggatatcct ttcactctgc atggttgaca |
| 1681 | gttctgccag cgcctgtgag agagaaggag atcacggctt ccccagatta tctggagata |
| 1741 | gctatttact gcataggggt cttcttaatc gcctgcatgg tggtgacagt catcttttgc |
| 1801 | cgaatgaaga ccacgaccaa gaagccagac ttcagcagcc agccagctgt gcacaagctg |
| 1861 | accaagcgca tccccctgcg gagacaggta acagtttcgg ccgagtccag ctcctccatg |
| 1921 | aactccaaca ccccgctggt gaggataaca acgcgtctgt cctcaacagc ggacaccccg |
| 1981 | atgctagcag gggtctccga gtatgagttg ccagaggatc caaagtggga attccccaga |
| 2041 | gataagctga cgctgggcaa acccctgggg gaaggttgct tcgggcaagt agtcatggct |
| 2101 | gaagcagtgg gaatcgataa agacaaaccc aaggaggcgg tcaccgtggc agtgaagatg |
| 2161 | ttgaaagatg atgccacaga gaaggacctg tctgatctgg tatcagagat ggagatgatg |
| 2221 | aagatgattg ggaaacataa gaacattatc aacctcctgg gggcctgcac gcaggatgga |
| 2281 | cctctctacg tcatagttga atatgcatcg aaaggcaacc tccgggaata cctccgagcc |
| 2341 | cggaggccac ctggcatgga gtactcctat gacattaacc gtgtccccga ggagcagatg |
| 2401 | accttcaagg acttggtgtc ctgcacctac cagctggcta gaggcatgga gtacttggct |
| 2461 | tcccaaaaat gtatccatcg agatttggct gccagaaacg tgttggtaac agaaaacaat |
| 2521 | gtgatgaaga tagcagactt tggcctggcc agggatatca acaacataga ctactataaa |
| 2581 | aagaccacaa atgggcgact tccagtcaag tggatggctc ctgaagccct ttttgataga |
| 2641 | gtttacactc atcagagcga tgtctggtcc ttcggggtgt taatgtggga gatctttact |
| 2701 | ttagggggct caccctaccc agggattccc gtggaggaac tttttaagct gctcaaagag |
| 2761 | ggacacagga tggacaagcc caccaactgc accaatgaac tgtacatgat gatgagggat |
| 2821 | tgctggcatg ctgtaccctc acagagaccc acattcaagc agttggtcga agacttggat |
| 2881 | cgaattctga ctctcacaac caatgaggaa tacttggatc tcacccagcc tctcgaacag |
| 2941 | tattctccta gttaccccga cacaagtagc tcttgttctt caggggacga ttctgtgttt |
| 3001 | tctccagacc ccatgcctta tgaaccctgt ctgcctcagt atccacacat aaacggcagt |
| 3061 | gttaaaacat gagtgaatgt gtcttcctgt ccccaaacag gacagcacca ggaacctact |
| 3121 | tacactgagc agagaggctg tgctccagag cctgtgacac gcctccactt gtatatatgg |
| 3181 | atcagaggag taaatagtgg gaagcatatt tgtcacgtgt gtaaagattt atacagttgg |
| 3241 | aacatgtact acaggaagga gactgttctg atagtgacag ccgccaccat gccacctttg |
| 3301 | accaca |
| SEQ ID NO: 8 | |
| FGFR2 IIIc | |
| From Mus musculus | |
| Gene No. 14183, Accession No. M86441 | |
| polypeptide, translation of SEQ ID NO: 7 | |
| MVSWGRFICLVLVTMATLSLARPSFSLVEDTTLEPEEPPTKYQI | |
| SQPEAYVVAPGESLELQCMLKDAAVISWTKDGVHLGPNNRTVLIGEYLQIKGATPRDS | |
| GLYACTAARTVDSETWIFMVNVTDAISSGDDEDDTDSSEDVVSENRSNQRAPYWTNTE | |
| KMEKRLHACPAANTVKFRCPAGGNPTSTMRWLKNGKEFKQEHRIGGYKVRNQHWSLIM | |
| ESVVPSDKGNYTCLVENEYGSINHTYHLDVVERSPHRPILQAGLPANASTVVGGDVEF | |
| VCKVYSDAQPHIQWIKHVEKNGSKNGPDGLPYLKVLKAAGVNTTDKEIEVLYIRNVTF | |
| EDAGEYTCLAGNSIGISFHSAWLTVLPAPVREKEITASPDYLEIAIYCIGVFLIACMV | |
| VTVIFCRMKTTTKKPDFSSQPAVHKLTKRIPLRRQVTVSAESSSSMNSNTPLVRITTR | |
| LSSTADTPMLAGVSEYELPEDPKWEFPRDKLTLGKPLGEGCFGQVVMAEAVGIDKDKP | |
| KEAVTVAVKMLKDDATEKDLSDLVSEMEMMKMIGKHKNIINLLGACTQDGPLYVIVEY | |
| ASKGNLREYLRARRPPGMEYSYDINRVPEEQMTFKDLVSCTYQLARGMEYLASQKCIH | |
| RDLAARNVLVTENNVMKIADFGLARDINNIDYYKKTTNGRLPVKWMAPEALFDRVYTH | |
| QSDVWSEGVLMWEIFTLGGSPYPGIPVEELFKLLKEGHRMDKPTNCTNELYMMMRDCW | |
| HAVPSQRPTFKQLVEDLDRILTLTTNEEYLDLTQPLEQYSPSYPDTSSSCSSGDDSVF | |
| SPDPMPYEPCLPQYPHINGSVKT | |
| SEQ ID NO: 9 | |
| FGFR2 IIIb | |
| From Mus musculus | |
| Gene No. 14183, Accession No. M63503 | |
| nucleotide (mRNA), 3037 bp | |
| 1 | ggcgagggga gagagccggg agaggcgagc ggcggcgcgg caggcgcgga acgggcgcac |
| 61 | ggacgatcga acgcgcggcc gccagagctc cggcgcgggg gctgcctgtg tgttcctggc |
| 121 | ccggcgtggc gactgctctc cgggctggcg ggggccgggc gtgagcccgg gcctcagcgt |
| 181 | tcctgagcgc tgcgagtgtt cactactcgc cagcaaagtt tggagtaggc aacgccaagc |
| 241 | tccagtcctt tcttctgctg ctgcccagat ccgagagcag ctccggtgtc atgtcctagc |
| 301 | tgttctgcga tccccggcgc gcgtgaagcc tcggaacctt cgcgccggct gctacccaag |
| 361 | gaatcgttct ctttttggag ttttcctccg agatcatcgc ctgctccatc ccgatccact |
| 421 | ctgggctccg gcgcagaccg agcgcagagg agcgctgcca ttcaagtggc agccacagca |
| 481 | gcagcagcag cagcagtggg agcaggaaca gcagtaacaa cagcaacagc agcacagccg |
| 541 | cctcagagct ttggctcctg agccccctgt gggctgaagg cattgcaggt agcccatggt |
| 601 | ctcagaagaa gtgtgcagat gggattaccg tccacgtgga gatatggaag aggaccaggg |
| 661 | attggcactg tgaccatggt cagctggggg cgcttcatct gcctggtctt ggtcaccatg |
| 721 | gcaaccttgt ccctggcccg gccctccttc agtttagttg aggataccac tttagaacca |
| 781 | gaaggagcac cgtactggac caacaccgag aagatggaga agcggctcca cgctgtccct |
| 841 | gccgccaaca ctgtgaagtt ccgctgtccg gctgggggga atccaacgcc cacaatgagg |
| 901 | tggttaaaaa acgggaagga gtttaagcag gagcatcgca ttggaggcta taaggtacga |
| 961 | aaccagcact ggagccttat tatggaaagt gtggtcccgt cagacaaagg caactacacc |
| 1021 | tgcctggtgg agaatgaata cgggtccatc aaccacacct accacctcga tgtcgttgaa |
| 1081 | cggtcaccac accggcccat cctccaagct ggactgcctg caaatgcctc cacggtggtc |
| 1141 | ggaggggatg tggagtttgt ctgcaaggtt tacagcgatg cccagcccca catccagtgg |
| 1201 | atcaagcacg tggaaaagaa cggcagtaaa tacgggcctg atgggctgcc ctacctcaag |
| 1261 | gtcctgaagc actcggggat aaatagctcc aatgcagaag tgctggctct gttcaatgtg |
| 1321 | acggagatgg atgctgggga atatatatgt aaggtctcca attatatagg gcaggccaac |
| 1381 | cagtctgcct ggctcactgt cctgcccaaa cagcaagcgc ctgtgagaga gaaggagatc |
| 1441 | acggcttccc cagattatct ggagatagct atttactgca taggggtctt cttaatcgcc |
| 1501 | tgcatggtgg tgacagtcat cttttgccga atgaagacca cgaccaagaa gccagacttc |
| 1561 | agcagccagc cagctgtgca caagctgacc aagcgcatcc ccctgcggag acaggtaaca |
| 1621 | gtttcggccg agtccagctc ctccatgaac tccaacaccc cgctggtgag gataacaacg |
| 1681 | cgtctgtcct caacagcgga caccccgatg ctagcagggg tctccgagta tgagttgcca |
| 1741 | gaggatccaa agtgggaatt ccccagagat aagctgacgc tgggcaaacc cctgggggaa |
| 1801 | ggttgcttcg ggcaagtagt catggctgaa gcagtgggaa tcgataaaga caaacccaag |
| 1861 | gaggcggtca ccgtggcagt gaagatgttg aaagatgatg ccacagagaa ggacctgtct |
| 1921 | gatctggtat cagagatgga gatgatgaag atgattggga aacataagaa cattatcaac |
| 1981 | ctcctggggg cctgcacgca ggatggacct ctctacgtca tagttgaata tgcatcgaaa |
| 2041 | ggcaacctcc gggaatacct ccgagcccgg aggccacctg gcatggagta ctcctatgac |
| 2101 | attaaccgtg tccccgagga gcagatgacc ttcaaggact tggtgtcctg cacctaccag |
| 2161 | ctggctagag gcatggagta cttggcttcc caaaaatgta tccatcgaga tttggctgcc |
| 2221 | agaaacgtgt tggtaacaga aaacaatgtg atgaagatag cagactttgg cctggccagg |
| 2281 | gatatcaaca acatagacta ctataaaaag accacaaatg ggcgacttcc agtcaagtgg |
| 2341 | atggctcctg aagccctttt tgatagagtt tacactcatc agagcgatgt ctggtccttc |
| 2401 | ggggtgttaa tgtgggagat ctttacttta gggggctcac cctacccagg gattcccgtg |
| 2461 | gaggaacttt ttaagctgct caaagaggga cacaggatgg acaagcccac caactgcacc |
| 2521 | aatgaactgt acatgatgat gagggattgc tggcatgctg taccctcaca gagacccaca |
| 2581 | ttcaagcagt tggtcgaaga cttggatcga attctgactc tcacaaccaa tgaggaatac |
| 2641 | ttggatctca cccagcctct cgaacagtat tctcctagtt accccgacac aaggagctct |
| 2701 | tgttcttcag gggacgattc tgtgttttct ccagacccca tgccttatga accctgtctg |
| 2761 | cctcagtatc cacacataaa cggcagtgtt aaaacatgag tgaatgtgtc ttcctgtccc |
| 2821 | caaacaggac agcaccagga acctacttac actgagcaga gaggctgtct cagagcctgt |
| 2881 | gacacgcctc cacttgtata tatggatcag aggagtaaat agtgggaagc atattgtcac |
| 2941 | gtgtgtaaag atttatacag ttcggaaaca tgttacctaa ccaggaaagg aagactgttt |
| 3001 | tcctgataag tggacagccg caagccacca tgccacc |
| SEQ ID NO: 10 | |
| FGFR2 IIIb | |
| From Mus musculus | |
| Gene No. 14183, Accession No. M63503 | |
| polypeptide, translation of SEQ ID NO: 9 | |
| MVSWGRFICLVLVTMATLSLARPSFSLVEDTTLEPEGAPYWTNT | |
| EKMEKRLHAVPAANTVKFRCPAGGNPTPTMRWLKNGKEFKQEHRIGGYKVRNQHWSLI | |
| MESVVPSDKGNYTCLVENEYGSINHTYHLDVVERSPHRPILQAGLPANASTVVGGDVE | |
| FVCKVYSDAQPHIQWIKHVEKNGSKYGPDGLPYLKVLKHSGINSSNAEVLALFNVTEM | |
| DAGEYICKVSNYIGQANQSAWLTVLPKQQAPVREKEITASPDYLEIAIYCIGVFLIAC | |
| MVVTVIFCRMKTTTKKPDFSSQPAVHKLTKRIPLRRQVTVSAESSSSMNSNTPLVRIT | |
| TRLSSTADTPMLAGVSEYELPEDPKWEFPRDKLTLGKPLGEGCFGQVVMAEAVGIDKD | |
| KPKEAVTVAVKMLKDDATEKDLSDLVSEMEMMKMIGKHKNIINLLGACTQDGPLYVIV | |
| EYASKGNLREYLRARRPPGMEYSYDINRVPEEQMTFKDLVSCTYQLARGMEYLASQKC | |
| IHRDLAARNVLVTENNVMKIADFGLARDINNIDYYKKTTNGRLPVKWMAPEALFDRVY | |
| THQSDVWSFGVLMWEIFTLGGSPYPGIPVEELFKLLKEGHRMDKPTNCTNELYMMMRD | |
| CWHAVPSQRPTFKQLVEDLDRILTLTTNEEYLDLTQPLEQYSPSYPDTRSSCSSGDDS | |
| VFSPDPMPYEPCLPQYPHINGSVKT | |
| SEQ ID NO: 11 | |
| E-cadherin (also known as cadherin-1, cdh1) | |
| From Xenopus (Silurana) tropicalis | |
| Gene No. 779546, Accession No. XM_002935997 | |
| nucleotide (mRNA), 3344 bp | |
| 1 | agagcaggga agtacagcgc tgcgctacaa gaactgagca aacgagcaga aaagtacaca |
| 61 | ttcctgatcc ttcggtcttt ccaaaagtcc ccaatggggt cacacaggcc atggttactt |
| 121 | ggtgctgtgg tgctgctggc actccttcag gtacagggag gactggcaga atggacacag |
| 181 | tgtcaaatgg gattttccaa ggaaaggtac agcttttcgg tacctaagaa cttggagaca |
| 241 | gacaaagcac tgggtagagt gatctttaac agctgtgagg gaccagtgag aattcagttt |
| 301 | gcctctaaag atcctaattt tgaaattcac aaagatggca cagtttatgt taagaatcct |
| 361 | accaagatga aagacaacag aaaaacattc cgtgtcctgg cttgggagaa tcaaggtcat |
| 421 | gtatactcta ccagtgtaac cttgaaaggg gaagggcatc accataagca ggacatttct |
| 481 | tctgtgaaac attcccacca cccaaaatct gagactggtt taaaaagaca aaaaagagac |
| 541 | tgggtgattc caccaatcgt aacatctgag aatgaaaagg gcccatttcc caaacggctt |
| 601 | gtgcagatca agtccagtaa tgcaaaggaa atcaaggttt tttacagtat cacaggccag |
| 661 | ggtgccgata cccctccaga aggagtgttc actattggac gggaggatgg atggctaaat |
| 721 | gtgacacgac ctttggacag agaagccatt gatagttaca ctcttttttc tcatgctgtg |
| 781 | tcagtaaatg ggcaaaatgt ggaagatccc atggaaatcc aaattaaagt acaagatcag |
| 841 | aatgataatg acccagtttt cacacaggag gtctttgaag gctatgtgcc tgaagggtct |
| 901 | aagccaggta cgcccgtcat gactgtatct gcaacagatg ccgatgatgc tatagacatg |
| 961 | tacaatggtg tgattactta ctccattctc aaccaagacc ctaaagagcc caacaatcaa |
| 1021 | atgttcacta ttgattccca gtctgggttg atcagcgtag ttacaactgg attagacaga |
| 1081 | gagaaaatac cagtgtacac actgactatt caagctgcag atggagaatt tgggaaagat |
| 1141 | cgcacaacaa ctgcaaaagc tgtgatcatt gtgacagaca ccaatgataa ccctcctgtg |
| 1201 | tttaacccaa cgcaatacat tgcagaggtt cctgaaaatg aagttggata tgaggttgca |
| 1261 | cgtcttacgg taacagatgc agatattgaa gggtcagatg cctggaatgc tgtgtacaag |
| 1321 | atcattaaag gaaatgaggc tggctttttc agcatccaaa cagatattga caacattggg |
| 1381 | ctactgaaaa cagtgaaggg tctggactat gagctgaaga agcagtatat tctgtcagtc |
| 1441 | attgtgacaa acaaagctaa cttttctgtt ccactacaaa cttcaactgc aacggtcact |
| 1501 | gtaactgtca cagatgtgaa tgaggcccca gtatttgtac cagtgttgaa agacgtgtct |
| 1561 | gtgccagagg atctgcccag tggccaagtt gttgctacct ataccgcaca ggatccagac |
| 1621 | aaggaacaga accagaaaat aagttacttc attggaaatg acccagcagg gtgggtgtct |
| 1681 | gtgaacagag ataatgggat tgtcactgga aatggaaact tggatcggga atcaaagttt |
| 1741 | gtgctaaaca acacctacaa agtcataatc ttggccgctg acagtggcac tccttctgcc |
| 1801 | actgggactg gaacccttgt gcttaatctc attgatgtta atgataatgg cccatttttg |
| 1861 | gatccccaac aaaatagttt ctgccagaag gatccaggct ttcgtgtatt taatatcatt |
| 1921 | gacaaagatc tttaccctaa cacataccca tatacagtag acctgactgg tgaatccaat |
| 1981 | gaaaactgga ctgctacagt gacagaacag agtttacttg agctgagacc taaaaaggaa |
| 2041 | ctggatattg gacgatacga agttttgatc tcattgagag acaatcaggg actgacagat |
| 2101 | gtgacaaagc tacagattac aatctgtcaa tgtaatggtg accaaatgca atgtgaggaa |
| 2161 | aaggctgctc aagcaggagg tttggggata tcagccatag ttggaatcct tggagggatc |
| 2221 | ctagcgcttc ttttattgtt gttgctgctc ttactgtttg tacgacgaaa gaaagtggta |
| 2281 | aaagaacctt tattaccacc agaagatgag actcgggaca atgtattttt ctatgatgaa |
| 2341 | gaaggcggtg gtgaggaaga ccaggatttt gatctaagcc agcttcaccg tggtctagat |
| 2401 | gctcgtccag atataatccg taatgatgtc gttccagttt tagctgctcc ccagtatcga |
| 2461 | ccccgtcctg ccaatccaga tgaaattgga aatttcattg atgagaactt gcatgcagct |
| 2521 | gacaatgacc ccactgctcc tccatacgac tcgctccttg tgttcgatta cgaaggcagt |
| 2581 | ggctctgagg ccgcatcact cagctctctt aactcttcca actctgattt agatcaggat |
| 2641 | tacagtgctt tgaataactg gggacctcgt ttcaccaaac tggcagaaat gtatggagga |
| 2701 | gatgaggatt agaatgtgca ctgcaatacc attttgattc taaacagtaa actaaaaacc |
| 2761 | ataattgtgt atgcagtctt tggaattcac tttgttttct cctgctctta aaacagagat |
| 2821 | aaggactgct caaaagttac tcctcctgct tttgtaaaat cgttcaaaaa tattttatgt |
| 2881 | atatgtatat atgaaaaaat cgtatttttt gtactatttg tgttcttata tccctgcaat |
| 2941 | ttgtaataca agaggatctt tatctgctta attataaata taaaatgccc gatatgattc |
| 3001 | actatgattt taatgtgttg agaaatcttt ttttaaaaag gtttccagac acctgacgct |
| 3061 | tggaagggaa ttccataaaa atataattga attgggggga gattgtgttt tgccatggtc |
| 3121 | tgatatacat tttcatatat atacatatga tcattcacag agtacagtca acatttggaa |
| 3181 | tttgatgagc ttgctggtcg aactgaaaaa aaaatgtatt atagctgggg taaaaattaa |
| 3241 | tgtatgagct aaatggggca caattttgat atctctgcat ttgtatttta cttggcatgt |
| 3301 | atacttttgt aataaaataa agatatacat taatatacaa cata |
| SEQ ID NO: 12 | |
| E-cadherin (also known as cadherin-1, cdh1) | |
| From Xenopus (Silurana) tropicalis | |
| Gene No. 779546, Accession No. XM_002935997 | |
| polypeptide (translation of SEQ ID NO: 11), | |
| 872 amino acids | |
| MGSHRPWLLGAVVLLALLQVQGGLAEWTQCQMGFSKERYSFSVP | |
| KNLETDKALGRVIFNSCEGPVRIQFASKDPNFEIHKDGTVYVKNPTKMKDNRKTFRVL | |
| AWENQGHVYSTSVTLKGEGHHHKQDISSVKHSHHPKSETGLKRQKRDWVIPPIVTSEN | |
| EKGPFPKRLVQIKSSNAKEIKVFYSITGQGADTPPEGVFTIGREDGWLNVTRPLDREA | |
| IDSYTLFSHAVSVNGQNVEDPMEIQIKVQDQNDNDPVFTQEVFEGYVPEGSKPGTPVM | |
| TVSATDADDAIDMYNGVITYSILNQDPKEPNNQMFTIDSQSGLISVVTTGLDREKIPV | |
| YTLTIQAADGEFGKDRTTTAKAVIIVTDTNDNPPVFNPTQYIAEVPENEVGYEVARLT | |
| VTDADIEGSDAWNAVYKIIKGNEAGFFSIQTDIDNIGLLKTVKGLDYELKKQYILSVI | |
| VTNKANFSVPLQTSTATVTVTVTDVNEAPVFVPVLKDVSVPEDLPSGQVVATYTAQDP | |
| DKEQNQKISYFIGNDPAGWVSVNRDNGIVTGNGNLDRESKFVLNNTYKVIILAADSGT | |
| PSATGTGTLVLNLIDVNDNGPFLDPQQNSFCQKDPGFRVFNIIDKDLYPNTYPYTVDL | |
| TGESNENWTATVTEQSLLELRPKKELDIGRYEVLISLRDNQGLTDVTKLQITICQCNG | |
| DQMQCEEKAAQAGGLGISAIVGILGGILALLLLLLLLLLFVRRKKVVKEPLLPPEDET | |
| RDNVFFYDEEGGGEEDQDFDLSQLHRGLDARPDIIRNDVVPVLAAPQYRPRPANPDEI | |
| GNFIDENLHAADNDPTAPPYDSLLVFDYEGSGSEAASLSSLNSSNSDLDQDYSALNNW | |
| GPRFTKLAEMYGGDED | |
| SEQ ID NO: 13 | |
| Vimentin | |
| From homo sapiens | |
| Accession No. BC000163 | |
| nucleotide (mRNA), 1862 bp | |
| 1 | gtccccgcgc cagagacgca gccgcgctcc caccacccac acccaccgcg ccctcgttcg |
| 61 | cctcttctcc gggagccagt ccgcgccacc gccgccgccc aggccatcgc caccctccgc |
| 121 | agccatgtcc accaggtccg tgtcctcgtc ctcctaccgc aggatgttcg gcggcccggg |
| 181 | caccgcgagc cggccgagct ccagccggag ctacgtgact acgtccaccc gcacctacag |
| 241 | cctgggcagc gcgctgcgcc ccagcaccag ccgcagcctc tacgcctcgt ccccgggcgg |
| 301 | cgtgtatgcc acgcgctcct ctgccgtgcg cctgcggagc agcgtgcccg gggtgcggct |
| 361 | cctgcaggac tcggtggact tctcgctggc cgacgccatc aacaccgagt tcaagaacac |
| 421 | ccgcaccaac gagaaggtgg agctgcagga gctgaatgac cgcttcgcca actacatcga |
| 481 | caaggtgcgc ttcctggagc agcagaataa gatcctgctg gccgagctcg agcagctcaa |
| 541 | gggccaaggc aagtcgcgcc tgggggacct ctacgaggag gagatgcggg agctgcgccg |
| 601 | gcaggtggac cagctaacca acgacaaagc ccgcgtcgag gtggagcgcg acaacctggc |
| 661 | cgaggacatc atgcgcctcc gggagaaatt gcaggaggag atgcttcaga gagaggaagc |
| 721 | cgaaaacacc ctgcaatctt tcagacagga tgttgacaat gcgtctctgg cacgtcttga |
| 781 | ccttgaacgc aaagtggaat ctttgcaaga agagattgcc tttttgaaga aactccacga |
| 841 | agaggaaatc caggagctgc aggctcagat tcaggaacag catgtccaaa tcgatgtgga |
| 901 | tgtttccaag cctgacctca cggctgccct gcgtgacgta cgtcagcaat atgaaagtgt |
| 961 | ggctgccaag aacctgcagg aggcagaaga atggtacaaa tccaagtttg ctgacctctc |
| 1021 | tgaggctgcc aaccggaaca atgacgccct gcgccaggca aagcaggagt ccactgagta |
| 1081 | ccggagacag gtgcagtccc tcacctgtga agtggatgcc cttaaaggaa ccaatgagtc |
| 1141 | cctggaacgc cagatgcgtg aaatggaaga gaactttgcc gttgaagctg ctaactacca |
| 1201 | agacactatt ggccgcctgc aggatgagat tcagaatatg aaggaggaaa tggctcgtca |
| 1261 | ccttcgtgaa taccaagacc tgctcaatgt taagatggcc cttgacattg agattgccac |
| 1321 | ctacaggaag ctgctggaag gcgaggagag caggatttct ctgcctcttc caaacttttc |
| 1381 | ctccctgaac ctgagggaaa ctaatctgga ttcactccct ctggttgata cccactcaaa |
| 1441 | aaggacactt ctgattaaga cggttgaaac tagagatgga caggttatca acgaaacttc |
| 1501 | tcagcatcac gatgaccttg aataaaaatt gcacacactc agtgcagcaa tatattacca |
| 1561 | gcaagaataa aaaagaaatc catatcttaa agaaacagct ttcaagtgcc tttctgcagt |
| 1621 | ttttcaggag cgcaagatag atttggaata ggaataagct ctagttctta acaaccgaca |
| 1681 | ctcctacaag atttagaaaa aagtttacaa cataatctag tttacagaaa aatcttgtgc |
| 1741 | tagaatactt tttaaaaggt attttgaata ccattaaaac tgcttttttt tttccagcaa |
| 1801 | gtatccaacc aacttggttc tgcttcaata aatctttgga aaaactcaaa aaaaaaaaaa |
| 1861 | aa |

SEQ ID NO: 14
Vimentin
From homo sapiens
Accession No. BC000163
polypeptide (translation of SEQ ID NO: 13),
466 amino acids
MSTRSVSSSSYRRMFGGPGTASRPSSSRSYVTTSTRTYSLGSAL
RPSTSRSLYASSPGGVYATRSSAVRLRSSVPGVRLLQDSVDFSLADAINTEFKNTRTN
EKVELQELNDRFANYIDKVRFLEQQNKILLAELEQLKGQGKSRLGDLYEEEMRELRRQ
VDQLTNDKARVEVERDNLAEDIMRLREKLQEEMLQREEAENTLQSFRQDVDNASLARL
DLERKVESLQEEIAFLKKLHEEEIQELQAQIQEQHVQIDVDVSKPDLTAALRDVRQQY
ESVAAKNLQEAEEWYKSKFADLSEAANRNNDALRQAKQESTEYRRQVQSLTCEVDALK
GTNESLERQMREMEENFAVEAANYQDTIGRLQDEIQNMKEEMARHLREYQDLLNVKMA
LDIEIATYRKLLEGEESRISLPLPNFSSLNLRETNLDSLPLVDTHSKRTLLIKTVETR
DGQVINETSQHHDDLE
SEQ ID NO: 15
N-cadherin
From homo sapiens
CCDS ID No. CCDS11891.1
nucleotide, 2721 bp
ATGTGCCGGATAGCGGGAGCGCTGCGGACCCTGCTGCCGCTGCTGGCGGCCCTGCTTCAGGCGTCTGTAG
AGGCTTCTGGTGAAATCGCATTATGCAAGACTGGATTTCCTGAAGATGTTTACAGTGCAGTCTTATCGAA
GGATGTGCATGAAGGACAGCCTCTTCTCAATGTGAAGTTTAGCAACTGCAATGGAAAAAGAAAAGTACAA
TATGAGAGCAGTGAGCCTGCAGATTTTAAGGTGGATGAAGATGGCATGGTGTATGCCGTGAGAAGCTTTC
CACTCTCTTCTGAGCATGCCAAGTTCCTGATATATGCCCAAGACAAAGAGACCCAGGAAAAGTGGCAAGT
GGCAGTAAAATTGAGCCTGAAGCCAACCTTAACTGAGGAGTCAGTGAAGGAGTCAGCAGAAGTTGAAGAA
ATAGTGTTCCCAAGACAATTCAGTAAGCACAGTGGCCACCTACAAAGGCAGAAGAGAGACTGGGTCATCC
CTCCAATCAACTTGCCAGAAAACTCCAGGGGACCTTTTCCTCAAGAGCTTGTCAGGATCAGGTCTGATAG
AGATAAAAACCTTTCACTGCGGTACAGTGTAACTGGGCCAGGAGCTGACCAGCCTCCAACTGGTATCTTC
ATTATCAACCCCATCTCGGGTCAGCTGTCGGTGACAAAGCCCCTGGATCGCGAGCAGATAGCCCGGTTTC
ATTTGAGGGCACATGCAGTAGATATTAATGGAAATCAAGTGGAGAACCCCATTGACATTGTCATCAATGT
TATTGACATGAATGACAACAGACCTGAGTTCTTACACCAGGTTTGGAATGGGACAGTTCCTGAGGGATCA
AAGCCTGGAACATATGTGATGACCGTAACAGCAATTGATGCTGACGATCCCAATGCCCTCAATGGGATGT
TGAGGTACAGAATCGTGTCTCAGGCTCCAAGCACCCCTTCACCCAACATGTTTACAATCAACAATGAGAC
TGGTGACATCATCACAGTGGCAGCTGGACTTGATCGAGAAAAAGTGCAACAGTATACGTTAATAATTCAA
GCTACAGACATGGAAGGCAATCCCACATATGGCCTTTCAAACACAGCCACGGCCGTCATCACAGTGACAG
ATGTCAATGACAATCCTCCAGAGTTTACTGCCATGACGTTTTATGGTGAAGTTCCTGAGAACAGGGTAGA
CATCATAGTAGCTAATCTAACTGTGACCGATAAGGATCAACCCCATACACCAGCCTGGAACGCAGTGTAC
AGAATCAGTGGCGGAGATCCTACTGGACGGTTCGCCATCCAGACCGACCCAAACAGCAACGACGGGTTAG
TCACCGTGGTCAAACCAATCGACTTTGAAACAAATAGGATGTTTGTCCTTACTGTTGCTGCAGAAAATCA
AGTGCCATTAGCCAAGGGAATTCAGCACCCCCCTCAGTCAACTGCAACCGTGTCTGTTACAGTTATTGAC
GTAAATGAAAACCCTTATTTTGCCCCCAATCCTAAGATCATTCGCCAAGAAGAAGGGCTTCATGCCGGTA
CCATGTTGACAACATTCACTGCTCAGGACCCAGATCGATATATGCAGCAAAATATTAGATACACTAAATT
ATCTGATCCTGCCAATTGGCTAAAAATAGATCCTGTGAATGGACAAATAACTACAATTGCTGTTTTGGAC
CGAGAATCACCAAATGTGAAAAACAATATATATAATGCTACTTTCCTTGCTTCTGACAATGGAATTCCTC
CTATGAGTGGAACAGGAACGCTGCAGATCTATTTACTTGATATTAATGACAATGCCCCTCAAGTGTTACC
TCAAGAGGCAGAGACTTGCGAAACTCCAGACCCCAATTCAATTAATATTACAGCACTTGATTATGACATT
GATCCAAATGCTGGACCATTTGCTTTTGATCTTCCTTTATCTCCAGTGACTATTAAGAGAAATTGGACCA
TCACTCGGCTTAATGGTGATTTTGCTCAGCTTAATTTAAAGATAAAATTTCTTGAAGCTGGTATCTATGA
AGTTCCCATCATAATCACAGATTCGGGTAATCCTCCCAAATCAAATATTTCCATCCTGCGTGTGAAGGTT
TGCCAGTGTGACTCCAACGGGGACTGCACAGATGTGGACAGGATTGTGGGTGCGGGGCTTGGCACCGGTG
CCATCATTGCCATCCTGCTCTGCATCATCATCCTGCTTATCCTTGTGCTGATGTTTGTGGTATGGATGAA
ACGCCGGGATAAAGAACGCCAGGCCAAACAACTTTTAATTGATCCAGAAGATGATGTAAGAGATAATATT
TTAAAATATGATGAAGAAGGTGGAGGAGAAGAAGACCAGGACTATGACTTGAGCCAGCTGCAGCAGCCTG
ACACTGTGGAGCCTGATGCCATCAAGCCTGTGGGAATCCGACGAATGGATGAAAGACCCATCCACGCCGA
GCCCCAGTATCCGGTCCGATCTGCAGCCCCACACCCTGGAGACATTGGGGACTTCATTAATGAGGGCCTT
AAAGCGGCTGACAATGACCCCACAGCTCCACCATATGACTCCCTGTTAGTGTTTGACTATGAAGGCAGTG
GCTCCACTGCTGGGTCCTTGAGCTCCCTTAATTCCTCAAGTAGTGGTGGTGAGCAGGACTATGATTACCT
GAACGACTGGGGGCCACGGTTCAAGAAACTTGCTGACATGTATGGTGGAGGTGATGACTGA
SEQ ID NO: 16
N-cadherin
From homo sapiens
CCDS ID No. CCDS11891.1
polypeptide (translation of SEQ ID NO: 15), 906 amino acids
MCRIAGALRTLLPLLAALLQASVEASGEIALCKTGFPEDVYSAVLSKDVHEGQPLLNVKFSNCNGKRKVQ
YESSEPADFKVDEDGMVYAVRSFPLSSEHAKFLIYAQDKETQEKWQVAVKLSLKPTLTEESVKESAEVEE
IVFPRQFSKHSGHLQRQKRDWVIPPINLPENSRGPFPQELVRIRSDRDKNLSLRYSVTGPGADQPPTGIF
IINPISGQLSVTKPLDREQIARFHLRAHAVDINGNQVENPIDIVINVIDMNDNRPEFLHQVWNGTVPEGS
KPGTYVMTVTAIDADDPNALNGMLRYRIVSQAPSTPSPNMFTINNETGDIITVAAGLDREKVQQYTLIIQ
ATDMEGNPTYGLSNTATAVITVTDVNDNPPEFTAMTFYGEVPENRVDIIVANLTVTDKDQPHTPAWNAVY
RISGGDPTGRFAIQTDPNSNDGLVTVVKPIDFETNRMFVLTVAAENQVPLAKGIQHPPQSTATVSVTVID
VNENPYFAPNPKIIRQEEGLHAGTMLTTFTAQDPDRYMQQNIRYTKLSDPANWLKIDPVNGQITTIAVLD
RESPNVKNNIYNATFLASDNGIPPMSGTGTLQIYLLDINDNAPQVLPQEAETCETPDPNSINITALDYDI
DPNAGPFAFDLPLSPVTIKRNWTITRLNGDFAQLNLKIKFLEAGIYEVPIIITDSGNPPKSNISILRVKV
CQCDSNGDCTDVDRIVGAGLGTGAIIAILLCIIILLILVLMFVVWMKRRDKERQAKQLLIDPEDDVRDNI
LKYDEEGGGEEDQDYDLSQLQQPDTVEPDAIKPVGIRRMDERPIHAEPQYPVRSAAPHPGDIGDFINEGL
KAADNDPTAPPYDSLLVFDYEGSGSTAGSLSSLNSSSSGGEQDYDYLNDWGPRFKKLADMYGGGDD
SEQ ID NO: 17
O-cadherin (also known as ob-cadherin)
From homo sapiens
CCDS ID No. CCDS10803.1
nucleotide, 2391 bp
ATGAAGGAGAACTACTGTTTACAAGCCGCCCTGGTGTGCCTGGGCATGCTGTGCCACAGCCATGCCTTTG
CCCCAGAGCGGCGGGGGCACCTGCGGCCCTCCTTCCATGGGCACCATGAGAAGGGCAAGGAGGGGCAGGT
GCTACAGCGCTCCAAGCGTGGCTGGGTCTGGAACCAGTTCTTCGTGATAGAGGAGTACACCGGGCCTGAC
CCCGTGCTTGTGGGCAGGCTTCATTCAGATATTGACTCTGGTGATGGGAACATTAAATACATTCTCTCAG
GGGAAGGAGCTGGAACCATTTTTGTGATTGATGACAAATCAGGGAACATTCATGCCACCAAGACGTTGGA
TCGAGAAGAGAGAGCCCAGTACACGTTGATGGCTCAGGCGGTGGACAGGGACACCAATCGGCCACTGGAG
CCACCGTCGGAATTCATTGTCAAGGTCCAGGACATTAATGACAACCCTCCGGAGTTCCTGCACGAGACCT
ATCATGCCAACGTGCCTGAGAGGTCCAATGTGGGAACGTCAGTAATCCAGGTGACAGCTTCAGATGCAGA
TGACCCCACTTATGGAAATAGCGCCAAGTTAGTGTACAGTATCCTCGAAGGACAACCCTATTTTTCGGTG
GAAGCACAGACAGGTATCATCAGAACAGCCCTACCCAACATGGACAGGGAGGCCAAGGAGGAGTACCACG
TGGTGATCCAGGCCAAGGACATGGGTGGACATATGGGCGGACTCTCAGGGACAACCAAAGTGACGATCAC
ACTGACCGATGTCAATGACAACCCACCAAAGTTTCCGCAGAGCGTATACCAGATGTCTGTGTCAGAAGCA
GCCGTCCCTGGGGAGGAAGTAGGAAGAGTGAAAGCTAAAGATCCAGACATTGGAGAAAATGGCTTAGTCA
CATACAATATTGTTGATGGAGATGGTATGGAATCGTTTGAAATCACAACGGACTATGAAACACAGGAGGG
GGTGATAAAGCTGAAAAAGCCTGTAGATTTTGAAACCAAAAGAGCCTATAGCTTGAAGGTAGAGGCAGCC
AACGTGCACATCGACCCGAAGTTTATCAGCAATGGCCCTTTCAAGGACACTGTGACCGTCAAGATCTCAG
TAGAAGATGCTGATGAGCCCCCTATGTTCTTGGCCCCAAGTTACATCCACGAAGTCCAAGAAAATGCAGC
TGCTGGCACCGTGGTTGGGAGAGTGCATGCCAAAGACCCTGATGCTGCCAACAGCCCGATAAGGTATTCC
ATCGATCGTCACACTGACCTCGACAGATTTTTCACTATTAATCCAGAGGATGGTTTTATTAAAACTACAA
AACCTCTGGATAGAGAGGAAACAGCCTGGCTCAACATCACTGTCTTTGCAGCAGAAATCCACAATCGGCA
TCAGGAAGCCAAAGTCCCAGTGGCCATTAGGGTCCTTGATGTCAACGATAATGCTCCCAAGTTTGCTGCC
CCTTATGAAGGTTTCATCTGTGAGAGTGATCAGACCAAGCCACTTTCCAACCAGCCAATTGTTACAATTA
GTGCAGATGACAAGGATGACACGGCCAATGGACCAAGATTTATCTTCAGCCTACCCCCTGAAATCATTCA
CAATCCAAATTTCACAGTCAGAGACAACCGAGATAACACAGCAGGCGTGTACGCCCGGCGTGGAGGGTTC
AGTCGGCAGAAGCAGGACTTGTACCTTCTGCCCATAGTGATCAGCGATGGCGGCATCCCGCCCATGAGTA
GCACCAACACCCTCACCATCAAAGTCTGCGGGTGCGACGTGAACGGGGCACTGCTCTCCTGCAACGCAGA
GGCCTACATTCTGAACGCCGGCCTGAGCACAGGCGCCCTGATCGCCATCCTCGCCTGCATCGTCATTCTC
CTGGTCATTGTAGTATTGTTTGTGACCCTGAGAAGGCAAAAGAAAGAACCACTCATTGTCTTTGAGGAAG
AAGATGTCCGTGAGAACATCATTACTTATGATGATGAAGGGGGTGGGGAAGAAGACACAGAAGCCTTTGA
TATTGCCACCCTCCAGAATCCTGATGGTATCAATGGATTTATCCCCCGCAAAGACATCAAACCTGAGTAT
CAGTACATGCCTAGACCTGGGCTCCGGCCAGCGCCCAACAGCGTGGATGTCGATGACTTCATCAACACGA
GAATACAGGAGGCAGACAATGACCCCACGGCTCCTCCTTATGACTCCATTCAAATCTACGGTTATGAAGG
CAGGGGCTCAGTGGCCGGGTCCCTGAGCTCCCTAGAGTCGGCCACCACAGATTCAGACTTGGACTATGAT
TATCTACAGAACTGGGGACCTCGTTTTAAGAAACTAGCAGATTTGTATGGTTCCAAAGACACTTTTGATG
ACGATTCTTAA
SEQ ID NO: 18
O-cadherin (also known as ob-cadherin)
From homo sapiens
CCDS ID No. CCDS10803.1
polypeptide (translation of SEQ ID NO: 17), 796 amino acids
MKENYCLQAALVCLGMLCHSHAFAPERRGHLRPSFHGHHEKGKEGQVLQRSKRGWVWNQFFVIEEYTGPD
PVLVGRLHSDIDSGDGNIKYILSGEGAGTIFVIDDKSGNIHATKTLDREERAQYTLMAQAVDRDTNRPLE
PPSEFIVKVQDINDNPPEFLHETYHANVPERSNVGTSVIQVTASDADDPTYGNSAKLVYSILEGQPYFSV
EAQTGIIRTALPNMDREAKEEYHVVIQAKDMGGHMGGLSGTTKVTITLTDVNDNPPKFPQSVYQMSVSEA
AVPGEEVGRVKAKDPDIGENGLVTYNIVDGDGMESFEITTDYETQEGVIKLKKPVDFETKRAYSLKVEAA
NVHIDPKFISNGPFKDTVTVKISVEDADEPPMFLAPSYIHEVQENAAAGTVVGRVHAKDPDAANSPIRYS
IDRHTDLDRFFTINPEDGFIKTTKPLDREETAWLNITVFAAEIHNRHQEAKVPVAIRVLDVNDNAPKFAA
PYEGFICESDQTKPLSNQPIVTISADDKDDTANGPRFIFSLPPEIIHNPNFTVRDNRDNTAGVYARRGGF
SRQKQDLYLLPIVISDGGIPPMSSTNTLTIKVCGCDVNGALLSCNAEAYILNAGLSTGALTATLACIVIL
LVIVVLFVTLRRQKKEPLIVFEEEDVRENIITYDDEGGGEEDTEAFDIATLQNPDGINGFIPRKDIKPEY
QYMPRPGLRPAPNSVDVDDFINTRIQEADNDPTAPPYDSIQIYGYEGRGSVAGSLSSLESATTDSDLDYD
YLQNWGPRFKKLADLYGSKDTFDDDS
SEQ ID NO: 19
CD133 (also known as PROM1)
From homo sapiens
CCDS ID No. CCDS47029.1
nucleotide, 2598 bp
ATGGCCCTCGTACTCGGCTCCCTGTTGCTGCTGGGGCTGTGCGGGAACTCCTTTTCAGGAGGGCAGCCTT
CATCCACAGATGCTCCTAAGGCTTGGAATTATGAATTGCCTGCAACAAATTATGAGACCCAAGACTCCCA
TAAAGCTGGACCCATTGGCATTCTCTTTGAACTAGTGCATATCTTTCTCTATGTGGTACAGCCGCGTGAT
TTCCCAGAAGATACTTTGAGAAAATTCTTACAGAAGGCATATGAATCCAAAATTGATTATGACAAGCCAG
AAACTGTAATCTTAGGTCTAAAGATTGTCTACTATGAAGCAGGGATTATTCTATGCTGTGTCCTGGGGCT
GCTGTTTATTATTCTGATGCCTCTGGTGGGGTATTTCTTTTGTATGTGTCGTTGCTGTAACAAATGTGGT
GGAGAAATGCACCAGCGACAGAAGGAAAATGGGCCCTTCCTGAGGAAATGCTTTGCAATCTCCCTGTTGG
TGATTTGTATAATAATAAGCATTGGCATCTTCTATGGTTTTGTGGCAAATCACCAGGTAAGAACCCGGAT
CAAAAGGAGTCGGAAACTGGCAGATAGCAATTTCAAGGACTTGCGAACTCTCTTGAATGAAACTCCAGAG
CAAATCAAATATATATTGGCCCAGTACAACACTACCAAGGACAAGGCGTTCACAGATCTGAACAGTATCA
ATTCAGTGCTAGGAGGCGGAATTCTTGACCGACTGAGACCCAACATCATCCCTGTTCTTGATGAGATTAA
GTCCATGGCAACAGCGATCAAGGAGACCAAAGAGGCGTTGGAGAACATGAACAGCACCTTGAAGAGCTTG
CACCAACAAAGTACACAGCTTAGCAGCAGTCTGACCAGCGTGAAAACTAGCCTGCGGTCATCTCTCAATG
ACCCTCTGTGCTTGGTGCATCCATCAAGTGAAACCTGCAACAGCATCAGATTGTCTCTAAGCCAGCTGAA
TAGCAACCCTGAACTGAGGCAGCTTCCACCCGTGGATGCAGAACTTGACAACGTTAATAACGTTCTTAGG
ACAGATTTGGATGGCCTGGTCCAACAGGGCTATCAATCCCTTAATGATATACCTGACAGAGTACAACGCC
AAACCACGACTGTCGTAGCAGGTATCAAAAGGGTCTTGAATTCCATTGGTTCAGATATCGACAATGTAAC
TCAGCGTCTTCCTATTCAGGATATACTCTCAGCATTCTCTGTTTATGTTAATAACACTGAAAGTTACATC
CACAGAAATTTACCTACATTGGAAGAGTATGATTCATACTGGTGGCTGGGTGGCCTGGTCATCTGCTCTC
TGCTGACCCTCATCGTGATTTTTTACTACCTGGGCTTACTGTGTGGCGTGTGCGGCTATGACAGGCATGC
CACCCCGACCACCCGAGGCTGTGTCTCCAACACCGGAGGCGTCTTCCTCATGGTTGGAGTTGGATTAAGT
TTCCTCTTTTGCTGGATATTGATGATCATTGTGGTTCTTACCTTTGTCTTTGGTGCAAATGTGGAAAAAC
TGATCTGTGAACCTTACACGAGCAAGGAATTATTCCGGGTTTTGGATACACCCTACTTACTAAATGAAGA
CTGGGAATACTATCTCTCTGGGAAGCTATTTAATAAATCAAAAATGAAGCTCACTTTTGAACAAGTTTAC
AGTGACTGCAAAAAAAATAGAGGCACTTACGGCACTCTTCACCTGCAGAACAGCTTCAATATCAGTGAAC
ATCTCAACATTAATGAGCATACTGGAAGCATAAGCAGTGAATTGGAAAGTCTGAAGGTAAATCTTAATAT
CTTTCTGTTGGGTGCAGCAGGAAGAAAAAACCTTCAGGATTTTGCTGCTTGTGGAATAGACAGAATGAAT
TATGACAGCTACTTGGCTCAGACTGGTAAATCCCCCGCAGGAGTGAATCTTTTATCATTTGCATATGATC
TAGAAGCAAAAGCAAACAGTTTGCCCCCAGGAAATTTGAGGAACTCCCTGAAAAGAGATGCACAAACTAT
TAAAACAATTCACCAGCAACGAGTCCTTCCTATAGAACAATCACTGAGCACTCTATACCAAAGCGTCAAG
ATACTTCAACGCACAGGGAATGGATTGTTGGAGAGAGTAACTAGGATTCTAGCTTCTCTGGATTTTGCTC
AGAACTTCATCACAAACAATACTTCCTCTGTTATTATTGAGGAAACTAAGAAGTATGGGAGAACAATAAT
AGGATATTTTGAACATTATCTGCAGTGGATCGAGTTCTCTATCAGTGAGAAAGTGGCATCGTGCAAACCT
GTGGCCACCGCTCTAGATACTGCTGTTGATGTCTTTCTGTGTAGCTACATTATCGACCCCTTGAATTTGT
TTTGGTTTGGCATAGGAAAAGCTACTGTATTTTTACTTCCGGCTCTAATTTTTGCGGTAAAACTGGCTAA
GTACTATCGTCGAATGGATTCGGAGGACGTGTACGATGATGTTGAAACTATACCCATGAAAAATATGGAA
AATGGTAATAATGGTTATCATAAAGATCATGTATATGGTATTCACAATCCTGTTATGACAAGCCCATCAC
AACATTGA
SEQ ID NO: 20
CD133 (also known as PROM1)
From homo sapiens
CCDS ID No. CCDS47029.1
poplypeptide (translation of SEQ ID NO: 19), 865 amino acids
MALVLGSLLLLGLCGNSFSGGQPSSTDAPKAWNYELPATNYETQDSHKAGPIGILFELVHIFLYVVQPRD
FPEDTLRKFLQKAYESKIDYDKPETVILGLKIVYYEAGIILCCVLGLLFIILMPLVGYFFCMCRCCNKCG
GEMHQRQKENGPFLRKCFAISLLVICIIISIGIFYGFVANHQVRTRIKRSRKLADSNFKDLRTLLNETPE
QIKYILAQYNTTKDKAFTDLNSINSVLGGGILDRLRPNIIPVLDEIKSMATAIKETKEALENMNSTLKSL
HQQSTQLSSSLTSVKTSLRSSLNDPLCLVHPSSETCNSIRLSLSQLNSNPELRQLPPVDAELDNVNNVLR
TDLDGLVQQGYQSLNDIPDRVQRQTTTVVAGIKRVLNSIGSDIDNVTQRLPIQDILSAFSVYVNNTESYI
HRNLPTLEEYDSYWWLGGLVICSLLTLIVIFYYLGLLCGVCGYDRHATPTTRGCVSNTGGVFLMVGVGLS
FLFCWILMIIVVLTFVFGANVEKLICEPYTSKELFRVLDTPYLLNEDWEYYLSGKLFNKSKMKLTFEQVY
SDCKKNRGTYGTLHLQNSFNISEHLNINEHTGSISSELESLKVNLNIFLLGAAGRKNLQDFAACGIDRMN
YDSYLAQTGKSPAGVNLLSFAYDLEAKANSLPPGNLRNSLKRDAQTIKTIHQQRVLPIEQSLSTLYQSVK
ILQRTGNGLLERVTRILASLDFAQNFITNNTSSVIIEETKKYGRTIIGYFEHYLQWIEFSISEKVASCKP
VATALDTAVDVFLCSYIIDPLNLFWFGIGKATVFLLPALIFAVKLAKYYRRMDSEDVYDDVETIPMKNME
NGNNGYHKDHVYGIHNPVMTSPSQH
SEQ ID NO: 21
FGFR2, isoform 1
From homo sapiens
CCDS ID No. CCDS31298.1
nucleotide, 2466 bp
ATGGTCAGCTGGGGTCGTTTCATCTGCCTGGTCGTGGTCACCATGGCAACCTTGTCCCTGGCCCGGCCCT
CCTTCAGTTTAGTTGAGGATACCACATTAGAGCCAGAAGAGCCACCAACCAAATACCAAATCTCTCAACC
AGAAGTGTACGTGGCTGCGCCAGGGGAGTCGCTAGAGGTGCGCTGCCTGTTGAAAGATGCCGCCGTGATC
AGTTGGACTAAGGATGGGGTGCACTTGGGGCCCAACAATAGGACAGTGCTTATTGGGGAGTACTTGCAGA
TAAAGGGCGCCACGCCTAGAGACTCCGGCCTCTATGCTTGTACTGCCAGTAGGACTGTAGACAGTGAAAC
TTGGTACTTCATGGTGAATGTCACAGATGCCATCTCATCCGGAGATGATGAGGATGACACCGATGGTGCG
GAAGATTTTGTCAGTGAGAACAGTAACAACAAGAGAGCACCATACTGGACCAACACAGAAAAGATGGAAA
AGCGGCTCCATGCTGTGCCTGCGGCCAACACTGTCAAGTTTCGCTGCCCAGCCGGGGGGAACCCAATGCC
AACCATGCGGTGGCTGAAAAACGGGAAGGAGTTTAAGCAGGAGCATCGCATTGGAGGCTACAAGGTACGA
AACCAGCACTGGAGCCTCATTATGGAAAGTGTGGTCCCATCTGACAAGGGAAATTATACCTGTGTAGTGG
AGAATGAATACGGGTCCATCAATCACACGTACCACCTGGATGTTGTGGAGCGATCGCCTCACCGGCCCAT
CCTCCAAGCCGGACTGCCGGCAAATGCCTCCACAGTGGTCGGAGGAGACGTAGAGTTTGTCTGCAAGGTT
TACAGTGATGCCCAGCCCCACATCCAGTGGATCAAGCACGTGGAAAAGAACGGCAGTAAATACGGGCCCG
ACGGGCTGCCCTACCTCAAGGTTCTCAAGGCCGCCGGTGTTAACACCACGGACAAAGAGATTGAGGTTCT
CTATATTCGGAATGTAACTTTTGAGGACGCTGGGGAATATACGTGCTTGGCGGGTAATTCTATTGGGATA
TCCTTTCACTCTGCATGGTTGACAGTTCTGCCAGCGCCTGGAAGAGAAAAGGAGATTACAGCTTCCCCAG
ACTACCTGGAGATAGCCATTTACTGCATAGGGGTCTTCTTAATCGCCTGTATGGTGGTAACAGTCATCCT
GTGCCGAATGAAGAACACGACCAAGAAGCCAGACTTCAGCAGCCAGCCGGCTGTGCACAAGCTGACCAAA
CGTATCCCCCTGCGGAGACAGGTAACAGTTTCGGCTGAGTCCAGCTCCTCCATGAACTCCAACACCCCGC
TGGTGAGGATAACAACACGCCTCTCTTCAACGGCAGACACCCCCATGCTGGCAGGGGTCTCCGAGTATGA
ACTTCCAGAGGACCCAAAATGGGAGTTTCCAAGAGATAAGCTGACACTGGGCAAGCCCCTGGGAGAAGGT
TGCTTTGGGCAAGTGGTCATGGCGGAAGCAGTGGGAATTGACAAAGACAAGCCCAAGGAGGCGGTCACCG
TGGCCGTGAAGATGTTGAAAGATGATGCCACAGAGAAAGACCTTTCTGATCTGGTGTCAGAGATGGAGAT
GATGAAGATGATTGGGAAACACAAGAATATCATAAATCTTCTTGGAGCCTGCACACAGGATGGGCCTCTC
TATGTCATAGTTGAGTATGCCTCTAAAGGCAACCTCCGAGAATACCTCCGAGCCCGGAGGCCACCCGGGA
TGGAGTACTCCTATGACATTAACCGTGTTCCTGAGGAGCAGATGACCTTCAAGGACTTGGTGTCATGCAC
CTACCAGCTGGCCAGAGGCATGGAGTACTTGGCTTCCCAAAAATGTATTCATCGAGATTTAGCAGCCAGA
AATGTTTTGGTAACAGAAAACAATGTGATGAAAATAGCAGACTTTGGACTCGCCAGAGATATCAACAATA
TAGACTATTACAAAAAGACCACCAATGGGCGGCTTCCAGTCAAGTGGATGGCTCCAGAAGCCCTGTTTGA
TAGAGTATACACTCATCAGAGTGATGTCTGGTCCTTCGGGGTGTTAATGTGGGAGATCTTCACTTTAGGG
GGCTCGCCCTACCCAGGGATTCCCGTGGAGGAACTTTTTAAGCTGCTGAAGGAAGGACACAGAATGGATA
AGCCAGCCAACTGCACCAACGAACTGTACATGATGATGAGGGACTGTTGGCATGCAGTGCCCTCCCAGAG
ACCAACGTTCAAGCAGTTGGTAGAAGACTTGGATCGAATTCTCACTCTCACAACCAATGAGGAATACTTG
GACCTCAGCCAACCTCTCGAACAGTATTCACCTAGTTACCCTGACACAAGAAGTTCTTGTTCTTCAGGAG
ATGATTCTGTTTTTTCTCCAGACCCCATGCCTTACGAACCATGCCTTCCTCAGTATCCACACATAAACGG
CAGTGTTAAAACATGA
SEQ ID NO: 22
FGFR2, isoform 1
From homo sapiens
CCDS ID No. CCDS31298.1
polypeptide (translation of SEQ ID NO: 21), 821 amino acids
MVSWGRFICLVVVTMATLSLARPSFSLVEDTTLEPEEPPTKYQISQPEVYVAAPGESLEVRCLLKDAAVI
SWTKDGVHLGPNNRTVLIGEYLQIKGATPRDSGLYACTASRTVDSETWYFMVNVTDAISSGDDEDDTDGA
EDFVSENSNNKRAPYWTNTEKMEKRLHAVPAANTVKFRCPAGGNPMPTMRWLKNGKEFKQEHRIGGYKVR
NQHWSLIMESVVPSDKGNYTCVVENEYGSINHTYHLDVVERSPHRPILQAGLPANASTVVGGDVEFVCKV
YSDAQPHIQWIKHVEKNGSKYGPDGLPYLKVLKAAGVNTTDKEIEVLYIRNVTFEDAGEYTCLAGNSIGI
SFHSAWLTVLPAPGREKEITASPDYLEIAIYCIGVFLIACMVVTVILCRMKNTTKKPDFSSQPAVHKLTK
RIPLRRQVTVSAESSSSMNSNTPLVRITTRLSSTADTPMLAGVSEYELPEDPKWEFPRDKLTLGKPLGEG
CFGQVVMAEAVGIDKDKPKEAVTVAVKMLKDDATEKDLSDLVSEMEMMKMIGKHKNIINLLGACTQDGPL
YVIVEYASKGNLREYLRARRPPGMEYSYDINRVPEEQMTFKDLVSCTYQLARGMEYLASQKCIHRDLAAR
NVLVTENNVMKIADFGLARDINNIDYYKKTTNGRLPVKWMAPEALFDRVYTHQSDVWSFGVLMWEIFTLG
GSPYPGIPVEELFKLLKEGHRMDKPANCTNELYMMMRDCWHAVPSQRPTFKQLVEDLDRILTLTTNEEYL
DLSQPLEQYSPSYPDTRSSCSSGDDSVFSPDPMPYEPCLPQYPHINGSVKT
SEQ ID NO: 23
E-cadherin (also known as CDH1)
From homo sapiens
CCDS ID No. CCDS10869.1
nucleotide, 2649 bp
ATGGGCCCTTGGAGCCGCAGCCTCTCGGCGCTGCTGCTGCTGCTGCAGGTCTCCTCTTGGCTCTGCCAGG
AGCCGGAGCCCTGCCACCCTGGCTTTGACGCCGAGAGCTACACGTTCACGGTGCCCCGGCGCCACCTGGA
GAGAGGCCGCGTCCTGGGCAGAGTGAATTTTGAAGATTGCACCGGTCGACAAAGGACAGCCTATTTTTCC
CTCGACACCCGATTCAAAGTGGGCACAGATGGTGTGATTACAGTCAAAAGGCCTCTACGGTTTCATAACC
CACAGATCCATTTCTTGGTCTACGCCTGGGACTCCACCTACAGAAAGTTTTCCACCAAAGTCACGCTGAA
TACAGTGGGGCACCACCACCGCCCCCCGCCCCATCAGGCCTCCGTTTCTGGAATCCAAGCAGAATTGCTC
ACATTTCCCAACTCCTCTCCTGGCCTCAGAAGACAGAAGAGAGACTGGGTTATTCCTCCCATCAGCTGCC
CAGAAAATGAAAAAGGCCCATTTCCTAAAAACCTGGTTCAGATCAAATCCAACAAAGACAAAGAAGGCAA
GGTTTTCTACAGCATCACTGGCCAAGGAGCTGACACACCCCCTGTTGGTGTCTTTATTATTGAAAGAGAA
ACAGGATGGCTGAAGGTGACAGAGCCTCTGGATAGAGAACGCATTGCCACATACACTCTCTTCTCTCACG
CTGTGTCATCCAACGGGAATGCAGTTGAGGATCCAATGGAGATTTTGATCACGGTAACCGATCAGAATGA
CAACAAGCCCGAATTCACCCAGGAGGTCTTTAAGGGGTCTGTCATGGAAGGTGCTCTTCCAGGAACCTCT
GTGATGGAGGTCACAGCCACAGACGCGGACGATGATGTGAACACCTACAATGCCGCCATCGCTTACACCA
TCCTCAGCCAAGATCCTGAGCTCCCTGACAAAAATATGTTCACCATTAACAGGAACACAGGAGTCATCAG
TGTGGTCACCACTGGGCTGGACCGAGAGAGTTTCCCTACGTATACCCTGGTGGTTCAAGCTGCTGACCTT
CAAGGTGAGGGGTTAAGCACAACAGCAACAGCTGTGATCACAGTCACTGACACCAACGATAATCCTCCGA
TCTTCAATCCCACCACGTACAAGGGTCAGGTGCCTGAGAACGAGGCTAACGTCGTAATCACCACACTGAA
AGTGACTGATGCTGATGCCCCCAATACCCCAGCGTGGGAGGCTGTATACACCATATTGAATGATGATGGT
GGACAATTTGTCGTCACCACAAATCCAGTGAACAACGATGGCATTTTGAAAACAGCAAAGGGCTTGGATT
TTGAGGCCAAGCAGCAGTACATTCTACACGTAGCAGTGACGAATGTGGTACCTTTTGAGGTCTCTCTCAC
CACCTCCACAGCCACCGTCACCGTGGATGTGCTGGATGTGAATGAAGCCCCCATCTTTGTGCCTCCTGAA
AAGAGAGTGGAAGTGTCCGAGGACTTTGGCGTGGGCCAGGAAATCACATCCTACACTGCCCAGGAGCCAG
ACACATTTATGGAACAGAAAATAACATATCGGATTTGGAGAGACACTGCCAACTGGCTGGAGATTAATCC
GGACACTGGTGCCATTTCCACTCGGGCTGAGCTGGACAGGGAGGATTTTGAGCACGTGAAGAACAGCACG
TACACAGCCCTAATCATAGCTACAGACAATGGTTCTCCAGTTGCTACTGGAACAGGGACACTTCTGCTGA
TCCTGTCTGATGTGAATGACAACGCCCCCATACCAGAACCTCGAACTATATTCTTCTGTGAGAGGAATCC
AAAGCCTCAGGTCATAAACATCATTGATGCAGACCTTCCTCCCAATACATCTCCCTTCACAGCAGAACTA
ACACACGGGGCGAGTGCCAACTGGACCATTCAGTACAACGACCCAACCCAAGAATCTATCATTTTGAAGC
CAAAGATGGCCTTAGAGGTGGGTGACTACAAAATCAATCTCAAGCTCATGGATAACCAGAATAAAGACCA
AGTGACCACCTTAGAGGTCAGCGTGTGTGACTGTGAAGGGGCCGCTGGCGTCTGTAGGAAGGCACAGCCT
GTCGAAGCAGGATTGCAAATTCCTGCCATTCTGGGGATTCTTGGAGGAATTCTTGCTTTGCTAATTCTGA
TTCTGCTGCTCTTGCTGTTTCTTCGGAGGAGAGCGGTGGTCAAAGAGCCCTTACTGCCCCCAGAGGATGA
CACCCGGGACAACGTTTATTACTATGATGAAGAAGGAGGCGGAGAAGAGGACCAGGACTTTGACTTGAGC
CAGCTGCACAGGGGCCTGGACGCTCGGCCTGAAGTGACTCGTAACGACGTTGCACCAACCCTCATGAGTG
TCCCCCGGTATCTTCCCCGCCCTGCCAATCCCGATGAAATTGGAAATTTTATTGATGAAAATCTGAAAGC
GGCTGATACTGACCCCACAGCCCCGCCTTATGATTCTCTGCTCGTGTTTGACTATGAAGGAAGCGGTTCC
GAAGCTGCTAGTCTGAGCTCCCTGAACTCCTCAGAGTCAGACAAAGACCAGGACTATGACTACTTGAACG
AATGGGGCAATCGCTTCAAGAAGCTGGCTGACATGTACGGAGGCGGCGAGGACGACTAG
SEQ ID NO: 24
E-cadherin (also known as CDH1)
From homo sapiens
CCDS ID No. CCDS10869.1
polypeptide (translation of SEQ ID NO: 23), 882 amino acids
MGPWSRSLSALLLLLQVSSWLCQEPEPCHPGFDAESYTFTVPRRHLERGRVLGRVNFEDCTGRQRTAYFS
LDTRFKVGTDGVITVKRPLRFHNPQIHFLVYAWDSTYRKFSTKVTLNTVGHHHRPPPHQASVSGIQAELL
TFPNSSPGLRRQKRDWVIPPISCPENEKGPFPKNLVQIKSNKDKEGKVFYSITGQGADTPPVGVFIIERE
TGWLKVTEPLDRERIATYTLFSHAVSSNGNAVEDPMEILITVTDQNDNKPEFTQEVFKGSVMEGALPGTS
VMEVTATDADDDVNTYNAAIAYTILSQDPELPDKNMFTINRNTGVISVVTTGLDRESFPTYTLVVQAADL
QGEGLSTTATAVITVTDTNDNPPIFNPTTYKGQVPENEANVVITTLKVTDADAPNTPAWEAVYTILNDDG
GQFVVTTNPVNNDGILKTAKGLDFEAKQQYILHVAVTNVVPFEVSLTTSTATVTVDVLDVNEAPIFVPPE
KRVEVSEDFGVGQEITSYTAQEPDTFMEQKITYRIWRDTANWLEINPDTGAISTRAELDREDFEHVKNST
YTALIIATDNGSPVATGTGTLLLILSDVNDNAPIPEPRTIFFCERNPKPQVINIIDADLPPNTSPFTAEL
THGASANWTIQYNDPTQESIILKPKMALEVGDYKINLKLMDNQNKDQVTTLEVSVCDCEGAAGVCRKAQP
VEAGLQIPAILGILGGILALLILILLLLLFLRRRAVVKEPLLPPEDDTRDNVYYYDEEGGGEEDQDFDLS
QLHRGLDARPEVTRNDVAPTLMSVPRYLPRPANPDEIGNFIDENLKAADTDPTAPPYDSLLVFDYEGSGS
EAASLSSLNSSESDKDQDYDYLNEWGNRFKKLADMYGGGEDD

1-29. (canceled)

30. A method for detecting a circulating tumor cell (CTC) in a biological sample, the method comprising detecting at least one epithelial mesenchymal transition (EMT) biomarker in the biological sample.

31. The method of claim 30, wherein the sample is a blood sample.

32. The method of claim 30, wherein the at least one EMT biomarker is vimentin, N-cadherin, O-cadherin, E-cadherin, FGFR2 splice variant isoforms, or CD133.

33. The method of claim 30, wherein the method is performed at the time of or prior to cancer metastasis.

34. The method of claim 30, wherein the at least one EMT biomarker is detected by flow cytometry, ferromagnetic enrichment, ferromagnetic sorting, or EMT antigen-antibody binding.

35. The method of claim 30, comprising detecting at least two EMT biomarkers.

36. A method for detecting cancer in a subject, the method comprising
detecting the presence of at least one EMT biomarker in a sample from the subject;
comparing the detected amount of the at least one EMT biomarker from the sample to a control sample; and
correlating the detected amount of the at least one EMT biomarker from the sample to the presence of CTCs in the sample,
wherein the presence of CTCs in the sample indicates the presence of cancer in the subject.

37. The method of claim 36, wherein the at least one EMT biomarker is vimentin, N-cadherin, O-cadherin, E-cadherin, FGFR2 splice variant isoforms, or CD133.

38. The method of claim 36, wherein the cancer is selected from prostate, colon, and breast cancer.

39. A method for monitoring progression of cancer in a subject undergoing therapeutic treatment, the method comprising:
detecting the number of CTCs based on the expression of at least one EMT biomarker in a first and a second sample taken from the subject at a first and a second time; and
comparing the first and second levels of expression,
wherein a detected difference in number of CTCs based on the level of expression of the at least one EMT biomarker in the first and second samples indicates a change in the progression of the cancer.

40. The method of claim 39, wherein an increase in the detected level of the at least one EMT biomarker in the second sample relative to the first sample indicates progression of the cancer.

41. The method of claim 39, wherein a decrease in the detected level of the at least one EMT biomarker in the second sample relative to the first sample indicates that the therapeutic treatment is effective.

42. The method of claim 41, wherein the decrease indicates remission of the cancer.

43. The method of claim 39, whereby no difference in the detected level of the at least one EMT biomarker in the second sample relative to the first sample indicates arrest or stability in the progression of the cancer.

44. The method of claim 41, wherein the at least one EMT biomarker is vimentin, N-cadherin, O-cadherin, E-cadherin, FGFR2 splice variant isoforms, or CD133.

45. The method of claim 41, wherein the cancer is selected from prostate, colon, and breast cancer.

46. A method of treating cancer in a subject comprising administering to the subject a cancer drug linked to an antibody that specifically binds at least one EMT biomarker.

47. The method of claim 46, wherein the at least one EMT biomarker is vimentin, N-cadherin, O-cadherin, E-cadherin, FGFR2 splice variant isoforms, or CD133.

48. The method of claim 46, wherein the cancer is selected from prostate, colon, and breast cancer.

49. A kit for detecting a circulating tumor cell (CTC) in a biological sample, the kit comprising an antibody to at least one EMT biomarker and instructions for use.

50. The kit of claim 49, wherein the antibody is linked to a fluorescent reporter molecule, radionuclide, enzyme, or magnetic bead.

51. The kit of claim 49, wherein the at least one EMT biomarker is vimentin, N-cadherin, O-cadherin, E-cadherin, FGFR2 splice variant isoforms, or CD133.


SEQUENCE LISTING (24 sequences)

SEQ ID NO: 1 - DNA, 4321 bp, Mus musculus
cacacacacacgcacacacacacacacacacacttctcggcgcgcacgacgcccgcccttctccccgccccctccccagctccttgatctcccgtctgttttattactcctggtgcgagtccggcggactccgaggcccgctatttgttaccaactcgctctcattggcggggaggagagcagcggagaagggggtggggaggggaggggaagggaaggggtggccactgccggagccgactccgcgctgctgttggtgccgctgccgcttctgctgcctctgctgccgccgccgccgcctccggctcctcgctcggcccctctccgcctccatgtgccggatagcgggagcgccgcggaccctgctgccgcttctggcggccttgcttcaggcgtctgtggaggcttctggtgaaattgcattatgcaagactggatttcctgaagatgtttacagcgcagtcttaccgaaggatgtgcacgaaggacagccccttctcaatgtgaaattcagcaactgcaatagaaaaaggaaagttcagtatgaaagcagcgagccagcagatttcaaggtggacgaggacggcacggtgtatgctgtgagaagcttccctctcactgcagagcaggcaaagttcctgatatatgcccaagacaaagaaacccaggaaaagtggcaggtagctgtaaacctgagccgggagccaaccctgactgaggagcctatgaaggaaccacatgaaattgaagaaatagtattccctagacaacttgccaagcacagtggagctctacaaaggcagaagagagactgggtcatcccgccaatcaacttgccagaaaactccagaggaccctttcctcaagagcttgtcagaatcaggtctgatagagataaaaacctttccctgagatacagcgtcactgggccaggagctgaccagcctccaacgggcatcttcattatcaaccccatctcaggacagctgtcagtcacaaagcctctggatcgagagctgatagcccggtttcacttgagagcacatgcagtggacatcaatggcaatcaagtggagaaccccattgacattgtcatcaatgttattgacatgaatgataacagacctgagtttctgcaccaggtttggaatgggtctgttccagagggatcaaagcctgggacgtatgtgatgacggtcactgccattgatgcggatgatccaaatgccctgaatggaatgctgcggtacaggatcctgtcccaggcgcccagcacaccttcacccaacatgtttacaatcaacaatgagactggggacatcatcactgtggcagctggtctggatcgagagaaagtgcaacagtatacgttaataattcaagccacagacatggaaggcaatcccacttatggcctttcaaacacagccacagccgtcatcacggtgacagatgtcaatgacaatcctccagagtttactgccatgactttctacggagaagtccctgagaacagggtggacgtcattgtagccaacctaactgtcacggacaaagatcagccccacacgccggcctggaatgcggcatacagaatcagtggtggagaccctacaggaaggtttgccatcctgacagaccccaacagcaatgatgggctagtcacagtggtaaaaccaattgactttgaaacgaataggatgtttgtccttactgttgctgcagaaaaccaagtgccattagctaaaggcattcagcacccacctcagtcgacagccactgtgtctgtgacagttattgatgtcaatgaaaatccttattttgccccaaatcctaaaatcattcgccaagaggaaggcctccacgcaggtaccatgctgaccacgctcactgctcaggaccccgatcgatatatgcaacagaatatcagatacacaaaattgtctgatcctgccaactggctgaaaatagaccccgtgaatgggcagatcactactattgccgttttggacagagaatcgccaaatgtaaaaaacaacatctataatgctaccttccttgcttctgacaatggaatcccgcctatgagtgggacaggaacactgcaaatctatttacttgatatcaatgacaacgcccctcaggtgttacctcaagaggcggagacctgtgaaactccagaacccaactcaattaacatcacagcacttgattatgacatagacccaaacgccgggccgttcgcgtttgatcttcccttatctccagtgactattaaaagaaactggaccatcaaccggcttaatggtgattttgctcagctcaatttaaagataaaatttttggaagctggtatctatgaagttcccatcattatcacagattcagggaatccccccaagtccaacatttccatcctgcgtgtgaaagtttgtcagtgtgactccaatggagactgcacggacgtggacaggatcgtgggtgcagggcttggcacgggcgccatcatcgctatccttctgtgtatcatcatcctgctgatccttgttctcatgtttgtggtatggatgaaacggcgggataaagagcgccaagccaagcagcttttaattgacccagaagatgatgtaagagataatatattgaaatatgatgaagaaggtggaggagaagaagaccaggactatgacttgagccagctccagcaaccagatactgtggagcctgatgccatcaagcccgtgggaatcagacggctagacgagaggcctatccatgctgagccacagtacccagtccgatccgcagccccacaccctggggatattggggacttcattaatgagggccttaaagctgctgacaacgaccccacggcgccaccgtatgactccctcttagtctttgactacgagggcagcggctccacggctggctccttgagctccctcaactcctccagtagcggtggggaccaggactatgactacctgaatgactggggaccccgcttcaagaaactggcggacatgtacggcggtggtgacgactgaacggcaggacggacttggcttttggacaagtatgaacagtttcacctgatattcccaaaaaaaagcatacagaagctaggctttaactctgtagtccactagcaccgtgcttgctggaggctttggcgtaggctgcgaaccagtttgggctcccagggaatatcagtgatccaatactgtctggaaaacaccgagctcagctacacttgaattttacagtaaagaagcactgggatttatgtgcctttttgtacctttttcagattggaattagttttctgtttaaggctttaatggtactgatttctgaaatgataaggaaaagacaaaatattttgtggcgggagcagaaagttaaatgtgatacgcttcaacccacttttgttacaatgcatttgcttttgttaagatacagaacgaaacaaccagattaaaaaaaattaactcatggagtgattttgttacctttggggtgggggggatgagaccacaagataggaaaatgtacattacttctagttttagactttagatttttttttttcactaaaatcttaaaacttacgcagctggttgcagataaagggagttttcatatcaccaatttgtagcaaaatgaattttttcataaactagaatgttagacacattttggtcttaatccatgtacacttttttattttctgtattttttccacctcgctgtaaaaatggtgtgtgtacataatgtttatcagcatagactatggaggagtgcagagaactcggaacatgtgtatgtattatttggactttggattcaggttttttgcatgttaatatctttcgttatgggtaaagtatttacaaaacaaagtgacatttgattcaactgttgagctgtagttagaatactcaatttttaattttttaattttttttaaatttttttattttctttttgtttgtttcgttttggggaggggtaaaagttcttagcacaatgttttacataatttgtaccaaaaaaattacacacaaaaaaaaaaaaaagaaaagaaaagaaaagtgaaaggggtggcctgtttcttgcagcactagcaagtgtgtgtttttaaaaaacaaaacaaacaaacaaaaaaataaataaaaagaggaaaaagaaaaaaaaaaaagcttttaaactggagagacttctgaaacagctttgcgtctgtgttgtgtaccagaatacaaacaatacacctctgaccccagcgttctgaataaaaagctaattttggatctgg

SEQ ID NO: 2 - protein, 906 aa, Mus musculus
MCRIAGAPRTLLPLLAALLQASVEASGEIALCKTGFPEDVYSAVLPKDVHEGQPLLNVKFSNCNRKRKVQYESSEPADFKVDEDGTVYAVRSFPLTAEQAKFLIYAQDKETQEKWQVAVNLSREPTLTEEPMKEPHEIEEIVFPRQLAKHSGALQRQKRDWVIPPINLPENSRGPFPQELVRIRSDRDKNLSLRYSVTGPGADQPPTGIFIINPISGQLSVTKPLDRELIARFHLRAHAVDINGNQVENPIDIVINVIDMNDNRPEFLHQVWNGSVPEGSKPGTYVMTVTAIDADDPNALNGMLRYRILSQAPSTPSPNMFTINNETGDIITVAAGLDREKVQQYTLIIQATDMEGNPTYGLSNTATAVITVTDVNDNPPEFTAMTFYGEVPENRVDVIVANLTVTDKDQPHTPAWNAAYRISGGDPTGRFAILTDPNSNDGLVTVVKPIDFETNRMFVLTVAAENQVPLAKGIQHPPQSTATVSVTVIDVNENPYFAPNPKIIRQEEGLHAGTMLTTLTAQDPDRYMQQNIRYTKLSDPANWLKIDPVNGQITTIAVLDRESPNVKNNIYNATFLASDNGIPPMSGTGTLQIYLLDINDNAPQVLPQEAETCETPEPNSINITALDYDIDPNAGPFAFDLPLSPVTIKRNWTINRLNGDFAQLNLKIKFLEAGIYEVPIIITDSGNPPKSNISILRVKVCQCDSNGDCTDVDRIVGAGLGTGAIIAILLCIIILLILVLMFVVWMKRRDKERQAKQLLIDPEDDVRDNILKYDEEGGGEEDQDYDLSQLQQPDTVEPDAIKPVGIRRLDERPIHAEPQYPVRSAAPHPGDIGDFINEGLKAADNDPTAPPYDSLLVFDYEGSGSTAGSLSSLNSSSSGGDQDYDYLNDWGPRFKKLADMYGGGDD

SEQ ID NO: 3 - DNA, 3237 bp, Xenopus laevis
tcggcacgagctggagtgtacaggacttttaagatgctgctgggtgtctgcactgtgtccatgtgaatgtggcatttttattttgaattccctccggagacaagatttcatcaagagtttcctttggatattaagtcaaagtgcaagcaatggagattctctataagaaggcaataatctgggggatttactaaaattaaacaaacagattgacattcgctggatttatcaagcaattttgcatttacaacactaccaaaaatgaagaaagacttttgcttacacggtttacttttatgtttgggaattgcgtattgtagtcatgccacatctttaagaaaaaacaataaactaaggcaatcattccatggtcaccatgaaaaaggcaaagaagggcaagttttacataggtcaaagagaggatgggtttggaatcaattttttgtaatagaagaatacaccggaccagatcctgtactcgttggacggcttcactcagatgttgactctggagattggaagataaaatacatactctcaggagagggtgctgggaccatttttgtcattgatgacaaatcagggaatatccatgcaaccaagaccctggatcgagaagaaagggctcagtataccttaatggctcaggcagttgacagagaaacaaataaaccactggaaccaccatcagagtttatcgttaaagttcaagacataaatgataatcccccggagttcttgcatgaaaactaccacgcaaatgtgcctgagatgtccaatgtgggtacatcagtaattcaagtaacagcctctgatgcagatgatccaacatatggaaacagcgctaagcttgtgtatagtattctcgaagggcagccatatttttcagtcgaagcacaatcaggaatcattaggactgcccttccaaacatggacagagaagccaaggaagaataccatgttgttattcaagcaaaggatatgggaggacatatgggaggactctcagggacaactaaagtgacaataacgctgacagatgtcaatgacaatccaccaaagtttccacaaagtgcgtaccccatgtctgtgtcagaagctgctgtcccaggggaagaggttggcagaataaaagctaaagatccagacattggagaaaatggcttaataaagtaccgtattcttgaaggagatggggcagagatgtttgaaatcacagctgattatgtaactcaggaaggcgttgtaaagctaaaaaaggtggtggattatgaaaccaagaagttctacagtatgaaggttgaagctgtcaacgttcatattgatcccagattccttagccggggaccattcaaagacactgctactgttaagatctcagtagaggattttgatgaaccgcctattttcttagaaagaagttacattttggaagtatatgaaaatgctccatcggatactgtggtcggaagagtgcacgctaaagacccagatgctgctaacagcccaattaggtattcaatcgatcgccacactgaccttgacagattcttcagcatcaacccagaggatggtgtcatcaaaaccacaaagggtttggatagagaggaaagcccttggcacaacatctcagtcattgcaactgaagtccacaatcgaattcatgaaactagagttccagtagctattaaagtcttggataagaatgacaatgctccggaatttgcaaagccctatgaagcttttgtctgtgaaaatgctccaatcaatcaggagtttttgaccatcactgcagtagataaagatgatacagccaatggacttcgttttctctttagtttccccccagaaattgtacatccaaatccaaatttcaccataatagacaaacgagataacacagcaagcatccgtgttggccgtggagttttcagccgacagaaacaagacttgtatttggttcctattgttataagtgatgggggaagcccaccgatgagcagcaccaataccctttctgtccgaatctgcagttgcaatagtgatggatcccaactatcttgtaatgctgaaccccaatcccttaacgctggactcagtactggagcactgattgcaatccttgcttgcattgtaattttattagtgattgtggttttgtttgtgactctgaggagagagaagaaggaacctctaattgtctttgaagaggaagatatccgggaaaatataattacatatgatgatgaaggtggtggagaggaagacaccgaagcatttgacattgcaacactgcagaatcctgatgggattaatggatttatgccacggaaagatatcaaacccgaatttcaatataaccccagagatattggaataagaccagcaccaaacagtgttgacgttgatgacttcattaacacaaggatacatgaggccgataatgaccctgcagctccgccttatgactccattcagatctatggatacgaagggagaggttctgtggctggctctcttagttcattagagtcagcctctacagattcagatttggactatgattatctacaaaactggggacctcgatttaagaaactagcaaatttatatgggtccaaagacacttgtgaagatgattcttaacaaataagttctgaatttggccttatgaactgcataatgtactgaaatatccagagtaaacattaacaggtatttttttaaaggaaaacatgaaaaaggcttctttaaccttccaaggtttacaaacaggattccttccaaaacaagaactgttaaatggtggtggatactgtgaaaaccctatggcctgtgtagaagttgtgtattcatttttttttttgttttttgttttttttccaagaaaccacttgtaaaatgcagcctatttaagggaatggaaatgcaggaaaaacgcaacaaaaaaggggaatctttacagtattaaacataaccatcaaatcttctcaaacaaagcttccacacaaaaaaaaaaaaagataacagttttgagctgtaatttcgccttaaactatggacactttatatgtagtgcatttttaaacttgaaaaaaatatatatataatatccagccagcttcaatccatataatgtatgtacagtaaaatgtacaattattctgtctcttgagcatcagacttgttactgctgattcttgtaaatcttttttgcttataatcccctcgtgccga

SEQ ID NO: 4 - protein, 794 aa, Xenopus laevis
MKKDFCLHGLLLCLGIAYCSHATSLRKNNKLRQSFHGHHEKGKEGQVLHRSKRGWVWNQFFVIEEYTGPDPVLVGRLHSDVDSGDWKIKYILSGEGAGTIFVIDDKSGNIHATKTLDREERAQYTLMAQAVDRETNKPLEPPSEFIVKVQDINDNPPEFLHENYHANVPEMSNVGTSVIQVTASDADDPTYGNSAKLVYSILEGQPYFSVEAQSGIIRTALPNMDREAKEEYHVVIQAKDMGGHMGGLSGTTKVTITLTDVNDNPPKFPQSAYPMSVSEAAVPGEEVGRIKAKDPDIGENGLIKYRILEGDGAEMFEITADYVTQEGVVKLKKVVDYETKKFYSMKVEAVNVHIDPRFLSRGPFKDTATVKISVEDFDEPPIFLERSYILEVYENAPSDTVVGRVHAKDPDAANSPIRYSIDRHTDLDRFFSINPEDGVIKTTKGLDREESPWHNISVIATEVHNRIHETRVPVAIKVLDKNDNAPEFAKPYEAFVCENAPINQEFLTITAVDKDDTANGLRFLFSFPPEIVHPNPNFTIIDKRDNTASIRVGRGVFSRQKQDLYLVPIVISDGGSPPMSSTNTLSVRICSCNSDGSQLSCNAEPQSLNAGLSTGALIAILACIVILLVIVVLFVTLRREKKEPLIVFEEEDIRENIITYDDEGGGEEDTEAFDIATLQNPDGINGFMPRKDIKPEFQYNPRDIGIRPAPNSVDVDDFINTRIHEADNDPAAPPYDSIQIYGYEGRGSVAGSLSSLESASTDSDLDYDYLQNWGPRFKKLANLYGSKDTCEDDS

SEQ ID NO: 5 - DNA, 3701 bp, Mus musculus
gtccaatcagtgcgctcagactcagagccctaggctcctgctctttaaattaccgagccttgtggagaccccggcacctggccttaagctcagccctgaggatggtactttgagtgaatgaccaccttggagaccgttcttctgtttcccttgttaccagccaggaggcagaagagtccaccggtccaggaaagacccatttcccttgagtttccagaaagtacctcatgcttgagagatcaggccaacaactatggctctcgtcttcagtgccctgctgttactggggctgtgtggaaagatctcttcagaaggtcagcctgcattccataacactcctggggctatgaattatgaattgcctaccaccaaatatgagacccaagataccttcaatgctgggattgttggccctctctacaaaatggtgcacatcttcctcaacgtggtccagccgaatgacttccctctagatttgatcaaaaaactcatacagaacaagaactttgacatctcagttgattccaaggagccagaaatcatagtcttggctctgaagattgccctctatgagatcggagtccttatctgcgccatcctgggactgctgttcattatcctcatgcctctggtgggctgcttcttttgtatgtgccgttgctgcaacaaatgcggcggagagatgcaccagcggcagaagcagaatgcgccatgcaggaggaagtgcttgggcctctccctcctggtgatttgtctgctcatgagccttggcattatatatggctttgtggctaaccagcagaccaggactcggatcaaagggacccagaaactggcaaagagcaatttcagagactttcaaacactcctgactgaaacaccaaagcaaattgactatgtagtggagcagtacaccaacaccaagaacaaggcattctcagacctggatggcatcggctccgtgctgggaggcagaataaaggaccaactaaaacccaaagtaactcctgtcctcgaagagattaaggccatggcgacagccatcaaacagaccaaggatgccctgcagaacatgagcagcagcctgaaaagtctccaagatgcagccacccagctcaataccaacctgagctctgtgagaaacagcatcgagaattcgctcagcagcagtgactgtacctcagatccagccagcaagatctgcgatagcatcagaccaagcctaagcagtctggggagcagcctcaattcaagtcagctcccatcagtggatagagaactcaacactgttactgaagtcgacaaaactgatctggagagcctcgtcaaaagggggtatacgacaattgatgaaatacccaatacaatacaaaaccaaactgtggatgtcatcaaagacgtcaaaaataccttggactccattagctccaacattaaggacatgagccaaagtattcctattgaggatatgctgttacaggtctcccattaccttaataacagcaacagatacttaaaccaggagctgcccaagctggaagaatatgactcgtactggtggctgggtggcttgattgtctgctttctgctgactctcattgtgaccttctttttcctgggcttgctgtgtggtgtgtttggctatgacaagcatgccaccccaactagaagaggctgtgtgtccaacactggaggcatcttcctcatggctggggttggattcggcttccttttttgctggatattgatgatccttgtggttcttacgtttgttgttggtgcaaatgtggaaaagttgctctgcgaaccttatgaaaacaagaaattattacaggttttggacactccctatctgctcaaggaacaatggcaattttatctttctggcatgctattcaataacccagacattaacatgacctttgagcaagtctacagggattgcaaaagaggtcgaggtatatatgctgcttttcagcttgagaatgtcgtcaacgtcagtgatcatttcaacattgaccagatttctgaaaacataaatacggagttggaaaacctgaatgtgaacattgatagcattgaactgttggataacacaggaaggaagagcctcgaggactttgcacattctgggatagatacaatcgattattccacatacttgaaggagactgagaaatcccctactgaagtgaatctgctgacatttgcctctaccctggaagcaaaagcaaaccagttgcctgaaggaaagctgaaacaggccttcttactggatgtacagaatataagagccatccaccagcatctcctccctcctgtgcagcaatcactgaaatttgtgagggtgaggaatacgttaagacaaagtgtctggaccctccagcaaacaagcaacaagttgccggagaaagtgaagaagatccttgcctctttggactctgttcagcatttcctcaccaataacgtttccctcatcgttatcggggaaacgaagaagtttgggaaaacaatactaggctactttgaacattatctgcactgggtcttttatgccatcacagagaagatgacatcctgcaaacccatggccaccgcgatggactctgctgttaatggcattctgtgtggctatgttgcggaccctctgaatttgttctggttcggcatagggaaagccacggtgctcttacttccggctgtaatcattgctatcaagctggccaagtactatcgcaggatggattcagaggatgtatacgacgacccgtctcgatactgacaactggagttgaagctgcttgaacaacaagatagtcaacatggaaagcatcacagattttggatagtttctgagtcttctagaacgttccaagtgcagaagaaacctggtggagactcaggcgggcactaggaacatggcatcagtggtcttagggtagcactttgtcaggaatgaacagtcatcatggttataatccacatatccattgcaactcatgaatgattctctcctgttttgtttttaacttttctttttacactgattttctatttagacactaaaacatataggggtgcttattccccctggatacatttacctgtgaaccagctattccggtgtcatagctgggtacctaacttacttccatatgtgaagtgtgctaaacacaaaccagtttacagaagagatgtattttgtgtatagtaaactgtatatatacccttttaccacagtcagttttttaaacaaatgaatactctagatttttcttctaaatgaggttactgttggggtggttgtgacctagtgatgctgtagaaaggagtctgcattcactaaaagtgtgtcaacctagagcaggcaatgcccttccttgtggatttctgtctgctcgttttggagctacctgcggtttagaaatagaattcaagaacaatcacggagtttcccacttgatgccactgccaaagtcagaacaagggatcttgagagaaggaactgtcgctcagctgggagcggaatcattatcgcaatcacaggtcctggttcacagtttagtggcactctctggtttgtaagaatgggcattacgttcagtgtcatctggtcatctgtgatgtgtgtcatcagcctgtcctgatgttgagatttaaaataaagcatgaatgaacagaaaaaaaaaaaaaaaaaaaaa

SEQ ID NO: 6 - protein, 842 aa, Mus musculus
MALVFSALLLLGLCGKISSEGQPAFHNTPGAMNYELPTTKYETQDTFNAGIVGPLYKMVHIFLNVVQPNDFPLDLIKKLIQNKNFDISVDSKEPEIIVLALKIALYEIGVLICAILGLLFIILMPLVGCFFCMCRCCNKCGGEMHQRQKQNAPCRRKCLGLSLLVICLLMSLGIIYGFVANQQTRTRIKGTQKLAKSNFRDFQTLLTETPKQIDYVVEQYTNTKNKAFSDLDGIGSVLGGRIKDQLKPKVTPVLEEIKAMATAIKQTKDALQNMSSSLKSLQDAATQLNTNLSSVRNSIENSLSSSDCTSDPASKICDSIRPSLSSLGSSLNSSQLPSVDRELNTVTEVDKTDLESLVKRGYTTIDEIPNTIQNQTVDVIKDVKNTLDSISSNIKDMSQSIPIEDMLLQVSHYLNNSNRYLNQELPKLEEYDSYWWLGGLIVCFLLTLIVTFFFLGLLCGVFGYDKHATPTRRGCVSNTGGIFLMAGVGFGFLFCWILMILVVLTFVVGANVEKLLCEPYENKKLLQVLDTPYLLKEQWQFYLSGMLFNNPDINMTFEQVYRDCKRGRGIYAAFQLENVVNVSDHFNIDQISENINTELENLNVNIDSIELLDNTGRKSLEDFAHSGIDTIDYSTYLKETEKSPTEVNLLTFASTLEAKANQLPEGKLKQAFLLDVQNIRAIHQHLLPPVQQSLKFVRVRNTLRQSVWTLQQTSNKLPEKVKKILASLDSVQHFLTNNVSLIVIGETKKFGKTILGYFEHYLHWVFYAITEKMTSCKPMATAMDSAVNGILCGYVADPLNLFWFGIGKATVLLLPAVIIAIKLAKYYRRMDSEDVYDDPSRY

SEQ ID NO: 7 - DNA, 3306 bp, Mus musculus
gaattcccgcgcggccgccagagctccggcccgggggctgcctgtgtgttcctggcccggcgtggcgactgctctccgggctggcgggggccgggcgtgagcccgggcctcagcgttcctgagcgctgcgagtgttcactactcgccagcaaagtttggagtaggcaacgcaagctccagtcctttcttctgctgctgcccagatccgagagcagctccggtgtatgtctagctgttctgcgatcccggcgcgcgtgaagcctcggaaccttggcgccggctgctacccaaggaatcgttctctttttggagttttcctccgagatcatcgcctgctccatcccgatccactctgggctccggcgcagcaccgagcgcagaggagcgctgccattcaagtggcagccacagcagcagcagcagcagcagtgggagcaggaacagcagtaacaacagcaacagcagcacagccgcctcagagctttgctcctgagcccctgtgggctgaaggcattgcaggtagcccatggtctcagaagaagtgtgcagatgggattaccgtccacgtggagatatggaagaggaccagggattggcactgtgaccatggtcagctgggggcgcttcatctgcctggtcttggtcaccatggcaaccttgtccctggcccggccctccttcagtttagttgaggataccactttagaaccagaagagccaccaaccaaataccaaatctcccaaccagaagcgtacgtggttgcccccggggaatcgctagagttgcagtgcatgttgaaagatgccgccgtgatcagttggactaaggatggggtgcacttggggcccaacaataggacagtgcttattggggagtatctccagataaaaggtgccacacctagagactccggcctctatgcttgtactgcagctaggacggtagacagtgaaacttggatcttcatggtgaatgtcacagatgccatctcatctggagatgatgaggacgacacagatagctccgaagacgttgtcagtgagaacaggagcaaccagagagcaccgtactggaccaacaccgagaagatggagaagcggctccacgcttgtcctgccgccaacactgtgaagttccgctgtccggctggggggaatccaacgtccacaatgaggtggttaaaaaacgggaaggagtttaagcaggagcatcgcattggaggctataaggtacgaaaccagcactggagccttattatggaaagtgtggtcccgtcagacaaaggcaactacacctgcctggtggagaatgaatacgggtccatcaaccacacctaccacctggatgtcgttgaacgttcaccacaccgtcccatcctccaagctggactgcctgcaaatgcctccacggtggtcggaggggatgtggagtttgtctgcaaggtttacagcgatgcccagccccacatccagtggatcaagcacgtggaaaagaacggcagtaaaaacgggcctgatgggctgccctacctcaaggttctgaaagctgccggtgttaacaccacggacaaagagattgaggttctctatattcggaatgtaacttttgaggatgctggggaatatacgtgcttggcgggtaattctatcgggatatcctttcactctgcatggttgacagttctgccagcgcctgtgagagagaaggagatcacggcttccccagattatctggagatagctatttactgcataggggtcttcttaatcgcctgcatggtggtgacagtcatcttttgccgaatgaagaccacgaccaagaagccagacttcagcagccagccagctgtgcacaagctgaccaagcgcatccccctgcggagacaggtaacagtttcggccgagtccagctcctccatgaactccaacaccccgctggtgaggataacaacgcgtctgtcctcaacagcggacaccccgatgctagcaggggtctccgagtatgagttgccagaggatccaaagtgggaattccccagagataagctgacgctgggcaaacccctgggggaaggttgcttcgggcaagtagtcatggctgaagcagtgggaatcgataaagacaaacccaaggaggcggtcaccgtggcagtgaagatgttgaaagatgatgccacagagaaggacctgtctgatctggtatcagagatggagatgatgaagatgattgggaaacataagaacattatcaacctcctgggggcctgcacgcaggatggacctctctacgtcatagttgaatatgcatcgaaaggcaacctccgggaatacctccgagcccggaggccacctggcatggagtactcctatgacattaaccgtgtccccgaggagcagatgaccttcaaggacttggtgtcctgcacctaccagctggctagaggcatggagtacttggcttcccaaaaatgtatccatcgagatttggctgccagaaacgtgttggtaacagaaaacaatgtgatgaagatagcagactttggcctggccagggatatcaacaacatagactactataaaaagaccacaaatgggcgacttccagtcaagtggatggctcctgaagccctttttgatagagtttacactcatcagagcgatgtctggtccttcggggtgttaatgtgggagatctttactttagggggctcaccctacccagggattcccgtggaggaactttttaagctgctcaaagagggacacaggatggacaagcccaccaactgcaccaatgaactgtacatgatgatgagggattgctggcatgctgtaccctcacagagacccacattcaagcagttggtcgaagacttggatcgaattctgactctcacaaccaatgaggaatacttggatctcacccagcctctcgaacagtattctcctagttaccccgacacaagtagctcttgttcttcaggggacgattctgtgttttctccagaccccatgccttatgaaccctgtctgcctcagtatccacacataaacggcagtgttaaaacatgagtgaatgtgtcttcctgtccccaaacaggacagcaccaggaacctacttacactgagcagagaggctgtgctccagagcctgtgacacgcctccacttgtatatatggatcagaggagtaaatagtgggaagcatatttgtcacgtgtgtaaagatttatacagttggaacatgtactacaggaaggagactgttctgatagtgacagccgccaccatgccacctttgaccaca

SEQ ID NO: 8 - protein, 821 aa, Mus musculus
MVSWGRFICLVLVTMATLSLARPSFSLVEDTTLEPEEPPTKYQISQPEAYVVAPGESLELQCMLKDAAVISWTKDGVHLGPNNRTVLIGEYLQIKGATPRDSGLYACTAARTVDSETWIFMVNVTDAISSGDDEDDTDSSEDVVSENRSNQRAPYWTNTEKMEKRLHACPAANTVKFRCPAGGNPTSTMRWLKNGKEFKQEHRIGGYKVRNQHWSLIMESVVPSDKGNYTCLVENEYGSINHTYHLDVVERSPHRPILQAGLPANASTVVGGDVEFVCKVYSDAQPHIQWIKHVEKNGSKNGPDGLPYLKVLKAAGVNTTDKEIEVLYIRNVTFEDAGEYTCLAGNSIGISFHSAWLTVLPAPVREKEITASPDYLEIAIYCIGVFLIACMVVTVIFCRMKTTTKKPDFSSQPAVHKLTKRIPLRRQVTVSAESSSSMNSNTPLVRITTRLSSTADTPMLAGVSEYELPEDPKWEFPRDKLTLGKPLGEGCFGQVVMAEAVGIDKDKPKEAVTVAVKMLKDDATEKDLSDLVSEMEMMKMIGKHKNIINLLGACTQDGPLYVIVEYASKGNLREYLRARRPPGMEYSYDINRVPEEQMTFKDLVSCTYQLARGMEYLASQKCIHRDLAARNVLVTENNVMKIADFGLARDINNIDYYKKTTNGRLPVKWMAPEALFDRVYTHQSDVWSFGVLMWEIFTLGGSPYPGIPVEELFKLLKEGHRMDKPTNCTNELYMMMRDCWHAVPSQRPTFKQLVEDLDRILTLTTNEEYLDLTQPLEQYSPSYPDTSSSCSSGDDSVFSPDPMPYEPCLPQYPHINGSVKT

SEQ ID NO: 9 - DNA, 3037 bp, Mus musculus
ggcgaggggagagagccgggagaggcgagcggcggcgcggcaggcgcggaacgggcgcacggacgatcgaacgcgcggccgccagagctccggcgcgggggctgcctgtgtgttcctggcccggcgtggcgactgctctccgggctggcgggggccgggcgtgagcccgggcctcagcgttcctgagcgctgcgagtgttcactactcgccagcaaagtttggagtaggcaacgccaagctccagtcctttcttctgctgctgcccagatccgagagcagctccggtgtcatgtcctagctgttctgcgatccccggcgcgcgtgaagcctcggaaccttcgcgccggctgctacccaaggaatcgttctctttttggagttttcctccgagatcatcgcctgctccatcccgatccactctgggctccggcgcagaccgagcgcagaggagcgctgccattcaagtggcagccacagcagcagcagcagcagcagtgggagcaggaacagcagtaacaacagcaacagcagcacagccgcctcagagctttggctcctgagccccctgtgggctgaaggcattgcaggtagcccatggtctcagaagaagtgtgcagatgggattaccgtccacgtggagatatggaagaggaccagggattggcactgtgaccatggtcagctgggggcgcttcatctgcctggtcttggtcaccatggcaaccttgtccctggcccggccctccttcagtttagttgaggataccactttagaaccagaaggagcaccgtactggaccaacaccgagaagatggagaagcggctccacgctgtccctgccgccaacactgtgaagttccgctgtccggctggggggaatccaacgcccacaatgaggtggttaaaaaacgggaaggagtttaagcaggagcatcgcattggaggctataaggtacgaaaccagcactggagccttattatggaaagtgtggtcccgtcagacaaaggcaactacacctgcctggtggagaatgaatacgggtccatcaaccacacctaccacctcgatgtcgttgaacggtcaccacaccggcccatcctccaagctggactgcctgcaaatgcctccacggtggtcggaggggatgtggagtttgtctgcaaggtttacagcgatgcccagccccacatccagtggatcaagcacgtggaaaagaacggcagtaaatacgggcctgatgggctgccctacctcaaggtcctgaagcactcggggataaatagctccaatgcagaagtgctggctctgttcaatgtgacggagatggatgctggggaatatatatgtaaggtctccaattatatagggcaggccaaccagtctgcctggctcactgtcctgcccaaacagcaagcgcctgtgagagagaaggagatcacggcttccccagattatctggagatagctatttactgcataggggtcttcttaatcgcctgcatggtggtgacagtcatcttttgccgaatgaagaccacgaccaagaagccagacttcagcagccagccagctgtgcacaagctgaccaagcgcatccccctgcggagacaggtaacagtttcggccgagtccagctcctccatgaactccaacaccccgctggtgaggataacaacgcgtctgtcctcaacagcggacaccccgatgctagcaggggtctccgagtatgagttgccagaggatccaaagtgggaattccccagagataagctgacgctgggcaaacccctgggggaaggttgcttcgggcaagtagtcatggctgaagcagtgggaatcgataaagacaaacccaaggaggcggtcaccgtggcagtgaagatgttgaaagatgatgccacagagaaggacctgtctgatctggtatcagagatggagatgatgaagatgattgggaaacataagaacattatcaacctcctgggggcctgcacgcaggatggacctctctacgtcatagttgaatatgcatcgaaaggcaacctccgggaatacctccgagcccggaggccacctggcatggagtactcctatgacattaaccgtgtccccgaggagcagatgaccttcaaggacttggtgtcctgcacctaccagctggctagaggcatggagtacttggcttcccaaaaatgtatccatcgagatttggctgccagaaacgtgttggtaacagaaaacaatgtgatgaagatagcagactttggcctggccagggatatcaacaacatagactactataaaaagaccacaaatgggcgacttccagtcaagtggatggctcctgaagccctttttgatagagtttacactcatcagagcgatgtctggtccttcggggtgttaatgtgggagatctttactttagggggctcaccctacccagggattcccgtggaggaactttttaagctgctcaaagagggacacaggatggacaagcccaccaactgcaccaatgaactgtacatgatgatgagggattgctggcatgctgtaccctcacagagacccacattcaagcagttggtcgaagacttggatcgaattctgactctcacaaccaatgaggaatacttggatctcacccagcctctcgaacagtattctcctagttaccccgacacaaggagctcttgttcttcaggggacgattctgtgttttctccagaccccatgccttatgaaccctgtctgcctcagtatccacacataaacggcagtgttaaaacatgagtgaatgtgtcttcctgtccccaaacaggacagcaccaggaacctacttacactgagcagagaggctgtctcagagcctgtgacacgcctccacttgtatatatggatcagaggagtaaatagtgggaagcatattgtcacgtgtgtaaagatttatacagttcggaaacatgttacctaaccaggaaaggaagactgttttcctgataagtggacagccgcaagccaccatgccacc

SEQ ID NO: 10 - protein, 707 aa, Mus musculus
MVSWGRFICLVLVTMATLSLARPSFSLVEDTTLEPEGAPYWTNTEKMEKRLHAVPAANTVKFRCPAGGNPTPTMRWLKNGKEFKQEHRIGGYKVRNQHWSLIMESVVPSDKGNYTCLVENEYGSINHTYHLDVVERSPHRPILQAGLPANASTVVGGDVEFVCKVYSDAQPHIQWIKHVEKNGSKYGPDGLPYLKVLKHSGINSSNAEVLALFNVTEMDAGEYICKVSNYIGQANQSAWLTVLPKQQAPVREKEITASPDYLEIAIYCIGVFLIACMVVTVIFCRMKTTTKKPDFSSQPAVHKLTKRIPLRRQVTVSAESSSSMNSNTPLVRITTRLSSTADTPMLAGVSEYELPEDPKWEFPRDKLTLGKPLGEGCFGQVVMAEAVGIDKDKPKEAVTVAVKMLKDDATEKDLSDLVSEMEMMKMIGKHKNIINLLGACTQDGPLYVIVEYASKGNLREYLRARRPPGMEYSYDINRVPEEQMTFKDLVSCTYQLARGMEYLASQKCIHRDLAARNVLVTENNVMKIADFGLARDINNIDYYKKTTNGRLPVKWMAPEALFDRVYTHQSDVWSFGVLMWEIFTLGGSPYPGIPVEELFKLLKEGHRMDKPTNCTNELYMMMRDCWHAVPSQRPTFKQLVEDLDRILTLTTNEEYLDLTQPLEQYSPSYPDTRSSCSSGDDSVFSPDPMPYEPCLPQYPHINGSVKT

SEQ ID NO: 11 - DNA, 3344 bp, Xenopus tropicalis
agagcagggaagtacagcgctgcgctacaagaactgagcaaacgagcagaaaagtacacattcctgatccttcggtctttccaaaagtccccaatggggtcacacaggccatggttacttggtgctgtggtgctgctggcactccttcaggtacagggaggactggcagaatggacacagtgtcaaatgggattttccaaggaaaggtacagcttttcggtacctaagaacttggagacagacaaagcactgggtagagtgatctttaacagctgtgagggaccagtgagaattcagtttgcctctaaagatcctaattttgaaattcacaaagatggcacagtttatgttaagaatcctaccaagatgaaagacaacagaaaaacattccgtgtcctggcttgggagaatcaaggtcatgtatactctaccagtgtaaccttgaaaggggaagggcatcaccataagcaggacatttcttctgtgaaacattcccaccacccaaaatctgagactggtttaaaaagacaaaaaagagactgggtgattccaccaatcgtaacatctgagaatgaaaagggcccatttcccaaacggcttgtgcagatcaagtccagtaatgcaaaggaaatcaaggttttttacagtatcacaggccagggtgccgatacccctccagaaggagtgttcactattggacgggaggatggatggctaaatgtgacacgacctttggacagagaagccattgatagttacactcttttttctcatgctgtgtcagtaaatgggcaaaatgtggaagatcccatggaaatccaaattaaagtacaagatcagaatgataatgacccagttttcacacaggaggtctttgaaggctatgtgcctgaagggtctaagccaggtacgcccgtcatgactgtatctgcaacagatgccgatgatgctatagacatgtacaatggtgtgattacttactccattctcaaccaagaccctaaagagcccaacaatcaaatgttcactattgattcccagtctgggttgatcagcgtagttacaactggattagacagagagaaaataccagtgtacacactgactattcaagctgcagatggagaatttgggaaagatcgcacaacaactgcaaaagctgtgatcattgtgacagacaccaatgataaccctcctgtgtttaacccaacgcaatacattgcagaggttcctgaaaatgaagttggatatgaggttgcacgtcttacggtaacagatgcagatattgaagggtcagatgcctggaatgctgtgtacaagatcattaaaggaaatgaggctggctttttcagcatccaaacagatattgacaacattgggctactgaaaacagtgaagggtctggactatgagctgaagaagcagtatattctgtcagtcattgtgacaaacaaagctaacttttctgttccactacaaacttcaactgcaacggtcactgtaactgtcacagatgtgaatgaggccccagtatttgtaccagtgttgaaagacgtgtctgtgccagaggatctgcccagtggccaagttgttgctacctataccgcacaggatccagacaaggaacagaaccagaaaataagttacttcattggaaatgacccagcagggtgggtgtctgtgaacagagataatgggattgtcactggaaatggaaacttggatcgggaatcaaagtttgtgctaaacaacacctacaaagtcataatcttggccgctgacagtggcactccttctgccactgggactggaacccttgtgcttaatctcattgatgttaatgataatggcccatttttggatccccaacaaaatagtttctgccagaaggatccaggctttcgtgtatttaatatcattgacaaagatctttaccctaacacatacccatatacagtagacctgactggtgaatccaatgaaaactggactgctacagtgacagaacagagtttacttgagctgagacctaaaaaggaactggatattggacgatacgaagttttgatctcattgagagacaatcagggactgacagatgtgacaaagctacagattacaatctgtcaatgtaatggtgaccaaatgcaatgtgaggaaaaggctgctcaagcaggaggtttggggatatcagccatagttggaatccttggagggatcctagcgcttcttttattgttgttgctgctcttactgtttgtacgacgaaagaaagtggtaaaagaacctttattaccaccagaagatgagactcgggacaatgtatttttctatgatgaagaaggcggtggtgaggaagaccaggattttgatctaagccagcttcaccgtggtctagatgctcgtccagatataatccgtaatgatgtcgttccagttttagctgctccccagtatcgaccccgtcctgccaatccagatgaaattggaaatttcattgatgagaacttgcatgcagctgacaatgaccccactgctcctccatacgactcgctccttgtgttcgattacgaaggcagtggctctgaggccgcatcactcagctctcttaactcttccaactctgatttagatcaggattacagtgctttgaataactggggacctcgtttcaccaaactggcagaaatgtatggaggagatgaggattagaatgtgcactgcaataccattttgattctaaacagtaaactaaaaaccataattgtgtatgcagtctttggaattcactttgttttctcctgctcttaaaacagagataaggactgctcaaaagttactcctcctgcttttgtaaaatcgttcaaaaatattttatgtatatgtatatatgaaaaaatcgtattttttgtactatttgtgttcttatatccctgcaatttgtaatacaagaggatctttatctgcttaattataaatataaaatgcccgatatgattcactatgattttaatgtgttgagaaatctttttttaaaaaggtttccagacacctgacgcttggaagggaattccataaaaatataattgaattggggggagattgtgttttgccatggtctgatatacattttcatatatatacatatgatcattcacagagtacagtcaacatttggaatttgatgagcttgctggtcgaactgaaaaaaaaatgtattatagctggggtaaaaattaatgtatgagctaaatggggcacaattttgatatctctgcatttgtattttacttggcatgtatacttttgtaataaaataaagatatacattaatatacaacata

SEQ ID NO: 12 - protein, 872 aa, Xenopus tropicalis
MGSHRPWLLGAVVLLALLQVQGGLAEWTQCQMGFSKERYSFSVPKNLETDKALGRVIFNSCEGPVRIQFASKDPNFEIHKDGTVYVKNPTKMKDNRKTFRVLAWENQGHVYSTSVTLKGEGHHHKQDISSVKHSHHPKSETGLKRQKRDWVIPPIVTSENEKGPFPKRLVQIKSSNAKEIKVFYSITGQGADTPPEGVFTIGREDGWLNVTRPLDREAIDSYTLFSHAVSVNGQNVEDPMEIQIKVQDQNDNDPVFTQEVFEGYVPEGSKPGTPVMTVSATDADDAIDMYNGVITYSILNQDPKEPNNQMFTIDSQSGLISVVTTGLDREKIPVYTLTIQAADGEFGKDRTTTAKAVIIVTDTNDNPPVFNPTQYIAEVPENEVGYEVARLTVTDADIEGSDAWNAVYKIIKGNEAGFFSIQTDIDNIGLLKTVKGLDYELKKQYILSVIVTNKANFSVPLQTSTATVTVTVTDVNEAPVFVPVLKDVSVPEDLPSGQVVATYTAQDPDKEQNQKISYFIGNDPAGWVSVNRDNGIVTGNGNLDRESKFVLNNTYKVIILAADSGTPSATGTGTLVLNLIDVNDNGPFLDPQQNSFCQKDPGFRVFNIIDKDLYPNTYPYTVDLTGESNENWTATVTEQSLLELRPKKELDIGRYEVLISLRDNQGLTDVTKLQITICQCNGDQMQCEEKAAQAGGLGISAIVGILGGILALLLLLLLLLLFVRRKKVVKEPLLPPEDETRDNVFFYDEEGGGEEDQDFDLSQLHRGLDARPDIIRNDVVPVLAAPQYRPRPANPDEIGNFIDENLHAADNDPTAPPYDSLLVFDYEGSGSEAASLSSLNSSNSDLDQDYSALNNWGPRFTKLAEMYGGDED

SEQ ID NO: 13 - DNA, 1862 bp, Homo sapiens
gtccccgcgccagagacgcagccgcgctcccaccacccacacccaccgcgccctcgttcgcctcttctccgggagccagtccgcgccaccgccgccgcccaggccatcgccaccctccgcagccatgtccaccaggtccgtgtcctcgtcctcctaccgcaggatgttcggcggcccgggcaccgcgagccggccgagctccagccggagctacgtgactacgtccacccgcacctacagcctgggcagcgcgctgcgccccagcaccagccgcagcctctacgcctcgtccccgggcggcgtgtatgccacgcgctcctctgccgtgcgcctgcggagcagcgtgcccggggtgcggctcctgcaggactcggtggacttctcgctggccgacgccatcaacaccgagttcaagaacacccgcaccaacgagaaggtggagctgcaggagctgaatgaccgcttcgccaactacatcgacaaggtgcgcttcctggagcagcagaataagatcctgctggccgagctcgagcagctcaagggccaaggcaagtcgcgcctgggggacctctacgaggaggagatgcgggagctgcgccggcaggtggaccagctaaccaacgacaaagcccgcgtcgaggtggagcgcgacaacctggccgaggacatcatgcgcctccgggagaaattgcaggaggagatgcttcagagagaggaagccgaaaacaccctgcaatctttcagacaggatgttgacaatgcgtctctggcacgtcttgaccttgaacgcaaagtggaatctttgcaagaagagattgcctttttgaagaaactccacgaagaggaaatccaggagctgcaggctcagattcaggaacagcatgtccaaatcgatgtggatgtttccaagcctgacctcacggctgccctgcgtgacgtacgtcagcaatatgaaagtgtggctgccaagaacctgcaggaggcagaagaatggtacaaatccaagtttgctgacctctctgaggctgccaaccggaacaatgacgccctgcgccaggcaaagcaggagtccactgagtaccggagacaggtgcagtccctcacctgtgaagtggatgcccttaaaggaaccaatgagtccctggaacgccagatgcgtgaaatggaagagaactttgccgttgaagctgctaactaccaagacactattggccgcctgcaggatgagattcagaatatgaaggaggaaatggctcgtcaccttcgtgaataccaagacctgctcaatgttaagatggcccttgacattgagattgccacctacaggaagctgctggaaggcgaggagagcaggatttctctgcctcttccaaacttttcctccctgaacctgagggaaactaatctggattcactccctctggttgatacccactcaaaaaggacacttctgattaagacggttgaaactagagatggacaggttatcaacgaaacttctcagcatcacgatgaccttgaataaaaattgcacacactcagtgcagcaatatattaccagcaagaataaaaaagaaatccatatcttaaagaaacagctttcaagtgcctttctgcagtttttcaggagcgcaagatagatttggaataggaataagctctagttcttaacaaccgacactcctacaagatttagaaaaaagtttacaacataatctagtttacagaaaaatcttgtgctagaatactttttaaaaggtattttgaataccattaaaactgcttttttttttccagcaagtatccaaccaacttggttctgcttcaataaatctttggaaaaactcaaaaaaaaaaaaaaa

SEQ ID NO: 14 - protein, 466 aa, Homo sapiens
MSTRSVSSSSYRRMFGGPGTASRPSSSRSYVTTSTRTYSLGSALRPSTSRSLYASSPGGVYATRSSAVRLRSSVPGVRLLQDSVDFSLADAINTEFKNTRTNEKVELQELNDRFANYIDKVRFLEQQNKILLAELEQLKGQGKSRLGDLYEEEMRELRRQVDQLTNDKARVEVERDNLAEDIMRLREKLQEEMLQREEAENTLQSFRQDVDNASLARLDLERKVESLQEEIAFLKKLHEEEIQELQAQIQEQHVQIDVDVSKPDLTAALRDVRQQYESVAAKNLQEAEEWYKSKFADLSEAANRNNDALRQAKQESTEYRRQVQSLTCEVDALKGTNESLERQMREMEENFAVEAANYQDTIGRLQDEIQNMKEEMARHLREYQDLLNVKMALDIEIATYRKLLEGEESRISLPLPNFSSLNLRETNLDSLPLVDTHSKRTLLIKTVETRDGQVINETSQHHDDLE

SEQ ID NO: 15 - DNA, 2721 bp, Homo sapiens
atgtgccggatagcgggagcgctgcggaccctgctgccgctgctggcggccctgcttcaggcgtctgtagaggcttctggtgaaatcgcattatgcaagactggatttcctgaagatgtttacagtgcagtcttatcgaaggatgtgcatgaaggacagcctcttctcaatgtgaagtttagcaactgcaatggaaaaagaaaagtacaatatgagagcagtgagcctgcagattttaaggtggatgaagatggcatggtgtatgccgtgagaagctttccactctcttctgagcatgccaagttcctgatatatgcccaagacaaagagacccaggaaaagtggcaagtggcagtaaaattgagcctgaagccaaccttaactgaggagtcagtgaaggagtcagcagaagttgaagaaatagtgttcccaagacaattcagtaagcacagtggccacctacaaaggcagaagagagactgggtcatccctccaatcaacttgccagaaaactccaggggaccttttcctcaagagcttgtcaggatcaggtctgatagagataaaaacctttcactgcggtacagtgtaactgggccaggagctgaccagcctccaactggtatcttcattatcaaccccatctcgggtcagctgtcggtgacaaagcccctggatcgcgagcagatagcccggtttcatttgagggcacatgcagtagatattaatggaaatcaagtggagaaccccattgacattgtcatcaatgttattgacatgaatgacaacagacctgagttcttacaccaggtttggaatgggacagttcctgagggatcaaagcctggaacatatgtgatgaccgtaacagcaattgatgctgacgatcccaatgccctcaatgggatgttgaggtacagaatcgtgtctcaggctccaagcaccccttcacccaacatgtttacaatcaacaatgagactggtgacatcatcacagtggcagctggacttgatcgagaaaaagtgcaacagtatacgttaataattcaagctacagacatggaaggcaatcccacatatggcctttcaaacacagccacggccgtcatcacagtgacagatgtcaatgacaatcctccagagtttactgccatgacgttttatggtgaagttcctgagaacagggtagacatcatagtagctaatctaactgtgaccgataaggatcaaccccatacaccagcctggaacgcagtgtacagaatcagtggcggagatcctactggacggttcgccatccagaccgacccaaacagcaacgacgggttagtcaccgtggtcaaaccaatcgactttgaaacaaataggatgtttgtccttactgttgctgcagaaaatcaagtgccattagccaagggaattcagcacccccctcagtcaactgcaaccgtgtctgttacagttattgacgtaaatgaaaacccttattttgcccccaatcctaagatcattcgccaagaagaagggcttcatgccggtaccatgttgacaacattcactgctcaggacccagatcgatatatgcagcaaaatattagatacactaaattatctgatcctgccaattggctaaaaatagatcctgtgaatggacaaataactacaattgctgttttggaccgagaatcaccaaatgtgaaaaacaatatatataatgctactttccttgcttctgacaatggaattcctcctatgagtggaacaggaacgctgcagatctatttacttgatattaatgacaatgcccctcaagtgttacctcaagaggcagagacttgcgaaactccagaccccaattcaattaatattacagcacttgattatgacattgatccaaatgctggaccatttgcttttgatcttcctttatctccagtgactattaagagaaattggaccatcactcggcttaatggtgattttgctcagcttaatttaaagataaaatttcttgaagctggtatctatgaagttcccatcataatcacagattcgggtaatcctcccaaatcaaatatttccatcctgcgtgtgaaggtttgccagtgtgactccaacggggactgcacagatgtggacaggattgtgggtgcggggcttggcaccggtgccatcattgccatcctgctctgcatcatcatcctgcttatccttgtgctgatgtttgtggtatggatgaaacgccgggataaagaacgccaggccaaacaacttttaattgatccagaagatgatgtaagagataatattttaaaatatgatgaagaaggtggaggagaagaagaccaggactatgacttgagccagctgcagcagcctgacactgtggagcctgatgccatcaagcctgtgggaatccgacgaatggatgaaagacccatccacgccgagccccagtatccggtccgatctgcagccccacaccctggagacattggggacttcattaatgagggccttaaagcggctgacaatgaccccacagctccaccatatgactccctgttagtgtttgactatgaaggcagtggctccactgctgggtccttgagctcccttaattcctcaagtagtggtggtgagcaggactatgattacctgaacgactgggggccacggttcaagaaacttgctgacatgtatggtggaggtgatgactga

SEQ ID NO: 16 - protein, 906 aa, Homo sapiens
MCRIAGALRTLLPLLAALLQASVEASGEIALCKTGFPEDVYSAVLSKDVHEGQPLLNVKFSNCNGKRKVQYESSEPADFKVDEDGMVYAVRSFPLSSEHAKFLIYAQDKETQEKWQVAVKLSLKPTLTEESVKESAEVEEIVFPRQFSKHSGHLQRQKRDWVIPPINLPENSRGPFPQELVRIRSDRDKNLSLRYSVTGPGADQPPTGIFIINPISGQLSVTKPLDREQIARFHLRAHAVDINGNQVENPIDIVINVIDMNDNRPEFLHQVWNGTVPEGSKPGTYVMTVTAIDADDPNALNGMLRYRIVSQAPSTPSPNMFTINNETGDIITVAAGLDREKVQQYTLIIQATDMEGNPTYGLSNTATAVITVTDVNDNPPEFTAMTFYGEVPENRVDIIVANLTVTDKDQPHTPAWNAVYRISGGDPTGRFAIQTDPNSNDGLVTVVKPIDFETNRMFVLTVAAENQVPLAKGIQHPPQSTATVSVTVIDVNENPYFAPNPKIIRQEEGLHAGTMLTTFTAQDPDRYMQQNIRYTKLSDPANWLKIDPVNGQITTIAVLDRESPNVKNNIYNATFLASDNGIPPMSGTGTLQIYLLDINDNAPQVLPQEAETCETPDPNSINITALDYDIDPNAGPFAFDLPLSPVTIKRNWTITRLNGDFAQLNLKIKFLEAGIYEVPIIITDSGNPPKSNISILRVKVCQCDSNGDCTDVDRIVGAGLGTGAIIAILLCIIILLILVLMFVVWMKRRDKERQAKQLLIDPEDDVRDNILKYDEEGGGEEDQDYDLSQLQQPDTVEPDAIKPVGIRRMDERPIHAEPQYPVRSAAPHPGDIGDFINEGLKAADNDPTAPPYDSLLVFDYEGSGSTAGSLSSLNSSSSGGEQDYDYLNDWGPRFKKLADMYGGGDD

SEQ ID NO: 17 - DNA, 2391 bp, Homo sapiens
atgaaggagaactactgtttacaagccgccctggtgtgcctgggcatgctgtgccacagccatgcctttgccccagagcggcgggggcacctgcggccctccttccatgggcaccatgagaagggcaaggaggggcaggtgctacagcgctccaagcgtggctgggtctggaaccagttcttcgtgatagaggagtacaccgggcctgaccccgtgcttgtgggcaggcttcattcagatattgactctggtgatgggaacattaaatacattctctcaggggaaggagctggaaccatttttgtgattgatgacaaatcagggaacattcatgccaccaagacgttggatcgagaagagagagcccagtacacgttgatggctcaggcggtggacagggacaccaatcggccactggagccaccgtcggaattcattgtcaaggtccaggacattaatgacaaccctccggagttcctgcacgagacctatcatgccaacgtgcctgagaggtccaatgtgggaacgtcagtaatccaggtgacagcttcagatgcagatgaccccacttatggaaatagcgccaagttagtgtacagtatcctcgaaggacaaccctatttttcggtggaagcacagacaggtatcatcagaacagccctacccaacatggacagggaggccaaggaggagtaccacgtggtgatccaggccaaggacatgggtggacatatgggcggactctcagggacaaccaaagtgacgatcacactgaccgatgtcaatgacaacccaccaaagtttccgcagagcgtataccagatgtctgtgtcagaagcagccgtccctggggaggaagtaggaagagtgaaagctaaagatccagacattggagaaaatggcttagtcacatacaatattgttgatggagatggtatggaatcgtttgaaatcacaacggactatgaaacacaggagggggtgataaagctgaaaaagcctgtagattttgaaaccaaaagagcctatagcttgaaggtagaggcagccaacgtgcacatcgacccgaagtttatcagcaatggccctttcaaggacactgtgaccgtcaagatctcagtagaagatgctgatgagccccctatgttcttggccccaagttacatccacgaagtccaagaaaatgcagctgctggcaccgtggttgggagagtgcatgccaaagaccctgatgctgccaacagcccgataaggtattccatcgatcgtcacactgacctcgacagatttttcactattaatccagaggatggttttattaaaactacaaaacctctggatagagaggaaacagcctggctcaacatcactgtctttgcagcagaaatccacaatcggcatcaggaagccaaagtcccagtggccattagggtccttgatgtcaacgataatgctcccaagtttgctgccccttatgaaggtttcatctgtgagagtgatcagaccaagccactttccaaccagccaattgttacaattagtgcagatgacaaggatgacacggccaatggaccaagatttatcttcagcctaccccctgaaatcattcacaatccaaatttcacagtcagagacaaccgagataacacagcaggcgtgtacgcccggcgtggagggttcagtcggcagaagcaggacttgtaccttctgcccatagtgatcagcgatggcggcatcccgcccatgagtagcaccaacaccctcaccatcaaagtctgcgggtgcgacgtgaacggggcactgctctcctgcaacgcagaggcctacattctgaacgccggcctgagcacaggcgccctgatcgccatcctcgcctgcatcgtcattctcctggtcattgtagtattgtttgtgaccctgagaaggcaaaagaaagaaccactcattgtctttgaggaagaagatgtccgtgagaacatcattacttatgatgatgaagggggtggggaagaagacacagaagcctttgatattgccaccctccagaatcctgatggtatcaatggatttatcccccgcaaagacatcaaacctgagtatcagtacatgcctagacctgggctccggccagcgcccaacagcgtggatgtcgatgacttcatcaacacgagaatacaggaggcagacaatgaccccacggctcctccttatgactccattcaaatctacggttatgaaggcaggggctcagtggccgggtccctgagctccctagagtcggccaccacagattcagacttggactatgattatctacagaactggggacctcgttttaagaaactagcagatttgtatggttccaaagacacttttgatgacgattcttaa

SEQ ID NO: 18 - protein, 796 aa, Homo sapiens
MKENYCLQAALVCLGMLCHSHAFAPERRGHLRPSFHGHHEKGKEGQVLQRSKRGWVWNQFFVIEEYTGPDPVLVGRLHSDIDSGDGNIKYILSGEGAGTIFVIDDKSGNIHATKTLDREERAQYTLMAQAVDRDTNRPLEPPSEFIVKVQDINDNPPEFLHETYHANVPERSNVGTSVIQVTASDADDPTYGNSAKLVYSILEGQPYFSVEAQTGIIRTALPNMDREAKEEYHVVIQAKDMGGHMGGLSGTTKVTITLTDVNDNPPKFPQSVYQMSVSEAAVPGEEVGRVKAKDPDIGENGLVTYNIVDGDGMESFEITTDYETQEGVIKLKKPVDFETKRAYSLKVEAANVHIDPKFISNGPFKDTVTVKISVEDADEPPMFLAPSYIHEVQENAAAGTVVGRVHAKDPDAANSPIRYSIDRHTDLDRFFTINPEDGFIKTTKPLDREETAWLNITVFAAEIHNRHQEAKVPVAIRVLDVNDNAPKFAAPYEGFICESDQTKPLSNQPIVTISADDKDDTANGPRFIFSLPPEIIHNPNFTVRDNRDNTAGVYARRGGFSRQKQDLYLLPIVISDGGIPPMSSTNTLTIKVCGCDVNGALLSCNAEAYILNAGLSTGALIAILACIVILLVIVVLFVTLRRQKKEPLIVFEEEDVRENIITYDDEGGGEEDTEAFDIATLQNPDGINGFIPRKDIKPEYQYMPRPGLRPAPNSVDVDDFINTRIQEADNDPTAPPYDSIQIYGYEGRGSVAGSLSSLESATTDSDLDYDYLQNWGPRFKKLADLYGSKDTFDDDS

SEQ ID NO: 19 - DNA, 2598 bp, Homo sapiens
atggccctcgtactcggctccctgttgctgctggggctgtgcgggaactccttttcaggagggcagccttcatccacagatgctcctaaggcttggaattatgaattgcctgcaacaaattatgagacccaagactcccataaagctggacccattggcattctctttgaactagtgcatatctttctctatgtggtacagccgcgtgatttcccagaagatactttgagaaaattcttacagaaggcatatgaatccaaaattgattatgacaagccagaaactgtaatcttaggtctaaagattgtctactatgaagcagggattattctatgctgtgtcctggggctgctgtttattattctgatgcctctggtggggtatttcttttgtatgtgtcgttgctgtaacaaatgtggtggagaaatgcaccagcgacagaaggaaaatgggcccttcctgaggaaatgctttgcaatctccctgttggtgatttgtataataataagcattggcatcttctatggttttgtggcaaatcaccaggtaagaacccggatcaaaaggagtcggaaactggcagatagcaatttcaaggacttgcgaactctcttgaatgaaactccagagcaaatcaaatatatattggcccagtacaacactaccaaggacaaggcgttcacagatctgaacagtatcaattcagtgctaggaggcggaattcttgaccgactgagacccaacatcatccctgttcttgatgagattaagtccatggcaacagcgatcaaggagaccaaagaggcgttggagaacatgaacagcaccttgaagagcttgcaccaacaaagtacacagcttagcagcagtctgaccagcgtgaaaactagcctgcggtcatctctcaatgaccctctgtgcttggtgcatccatcaagtgaaacctgcaacagcatcagattgtctctaagccagctgaatagcaaccctgaactgaggcagcttccacccgtggatgcagaacttgacaacgttaataacgttcttaggacagatttggatggcctggtccaacagggctatcaatcccttaatgatatacctgacagagtacaacgccaaaccacgactgtcgtagcaggtatcaaaagggtcttgaattccattggttcagatatcgacaatgtaactcagcgtcttcctattcaggatatactctcagcattctctgtttatgttaataacactgaaagttacatccacagaaatttacctacattggaagagtatgattcatactggtggctgggtggcctggtcatctgctctctgctgaccctcatcgtgattttttactacctgggcttactgtgtggcgtgtgcggctatgacaggcatgccaccccgaccacccgaggctgtgtctccaacaccggaggcgtcttcctcatggttggagttggattaagtttcctcttttgctggatattgatgatcattgtggttcttacctttgtctttggtgcaaatgtggaaaaactgatctgtgaaccttacacgagcaaggaattattccgggttttggatacaccctacttactaaatgaagactgggaatactatctctctgggaagctatttaataaatcaaaaatgaagctcacttttgaacaagtttacagtgactgcaaaaaaaatagaggcacttacggcactcttcacctgcagaacagcttcaatatcagtgaacatctcaacattaatgagcatactggaagcataagcagtgaattggaaagtctgaaggtaaatcttaatatctttctgttgggtgcagcaggaagaaaaaaccttcaggattttgctgcttgtggaatagacagaatgaattatgacagctacttggctcagactggtaaatcccccgcaggagtgaatcttttatcatttgcatatgatctagaagcaaaagcaaacagtttgcccccaggaaatttgaggaactccctgaaaagagatgcacaaactattaaaacaattcaccagcaacgagtccttcctatagaacaatcactgagcactctataccaaagcgtcaagatacttcaacgcacagggaatggattgttggagagagtaactaggattctagcttctctggattttgctcagaacttcatcacaaacaatacttcctctgttattattgaggaaactaagaagtatgggagaacaataataggatattttgaacattatctgcagtggatcgagttctctatcagtgagaaagtggcatcgtgcaaacctgtggccaccgctctagatactgctgttgatgtctttctgtgtagctacattatcgaccccttgaatttgttttggtttggcataggaaaagctactgtatttttacttccggctctaatttttgcggtaaaactggctaagtactatcgtcgaatggattcggaggacgtgtacgatgatgttgaaactatacccatgaaaaatatggaaaatggtaataatggttatcataaagatcatgtatatggtattcacaatcctgttatgacaagcccatcacaacattga

SEQ ID NO: 20 - protein, 865 aa, Homo sapiens
MALVLGSLLLLGLCGNSFSGGQPSSTDAPKAWNYELPATNYETQDSHKAGPIGILFELVHIFLYVVQPRDFPEDTLRKFLQKAYESKIDYDKPETVILGLKIVYYEAGIILCCVLGLLFIILMPLVGYFFCMCRCCNKCGGEMHQRQKENGPFLRKCFAISLLVICIIISIGIFYGFVANHQVRTRIKRSRKLADSNFKDLRTLLNETPEQIKYILAQYNTTKDKAFTDLNSINSVLGGGILDRLRPNIIPVLDEIKSMATAIKETKEALENMNSTLKSLHQQSTQLSSSLTSVKTSLRSSLNDPLCLVHPSSETCNSIRLSLSQLNSNPELRQLPPVDAELDNVNNVLRTDLDGLVQQGYQSLNDIPDRVQRQTTTVVAGIKRVLNSIGSDIDNVTQRLPIQDILSAFSVYVNNTESYIHRNLPTLEEYDSYWWLGGLVICSLLTLIVIFYYLGLLCGVCGYDRHATPTTRGCVSNTGGVFLMVGVGLSFLFCWILMIIVVLTFVFGANVEKLICEPYTSKELFRVLDTPYLLNEDWEYYLSGKLFNKSKMKLTFEQVYSDCKKNRGTYGTLHLQNSFNISEHLNINEHTGSISSELESLKVNLNIFLLGAAGRKNLQDFAACGIDRMNYDSYLAQTGKSPAGVNLLSFAYDLEAKANSLPPGNLRNSLKRDAQTIKTIHQQRVLPIEQSLSTLYQSVKILQRTGNGLLERVTRILASLDFAQNFITNNTSSVIIEETKKYGRTIIGYFEHYLQWIEFSISEKVASCKPVATALDTAVDVFLCSYIIDPLNLFWFGIGKATVFLLPALIFAVKLAKYYRRMDSEDVYDDVETIPMKNMENGNNGYHKDHVYGIHNPVMTSPSQH

SEQ ID NO: 21 - DNA, 2466 bp, Homo sapiens
atggtcagctggggtcgtttcatctgcctggtcgtggtcaccatggcaaccttgtccctggcccggccctccttcagtttagttgaggataccacattagagccagaagagccaccaaccaaataccaaatctctcaaccagaagtgtacgtggctgcgccaggggagtcgctagaggtgcgctgcctgttgaaagatgccgccgtgatcagttggactaaggatggggtgcacttggggcccaacaataggacagtgcttattggggagtacttgcagataaagggcgccacgcctagagactccggcctctatgcttgtactgccagtaggactgtagacagtgaaacttggtacttcatggtgaatgtcacagatgccatctcatccggagatgatgaggatgacaccgatggtgcggaagattttgtcagtgagaacagtaacaacaagagagcaccatactggaccaacacagaaaagatggaaaagcggctccatgctgtgcctgcggccaacactgtcaagtttcgctgcccagccggggggaacccaatgccaaccatgcggtggctgaaaaacgggaaggagtttaagcaggagcatcgcattggaggctacaaggtacgaaaccagcactggagcctcattatggaaagtgtggtcccatctgacaagggaaattatacctgtgtagtggagaatgaatacgggtccatcaatcacacgtaccacctggatgttgtggagcgatcgcctcaccggcccatcctccaagccggactgccggcaaatgcctccacagtggtcggaggagacgtagagtttgtctgcaaggtttacagtgatgcccagccccacatccagtggatcaagcacgtggaaaagaacggcagtaaatacgggcccgacgggctgccctacctcaaggttctcaaggccgccggtgttaacaccacggacaaagagattgaggttctctatattcggaatgtaacttttgaggacgctggggaatatacgtgcttggcgggtaattctattgggatatcctttcactctgcatggttgacagttctgccagcgcctggaagagaaaaggagattacagcttccccagactacctggagatagccatttactgcataggggtcttcttaatcgcctgtatggtggtaacagtcatcctgtgccgaatgaagaacacgaccaagaagccagacttcagcagccagccggctgtgcacaagctgaccaaacgtatccccctgcggagacaggtaacagtttcggctgagtccagctcctccatgaactccaacaccccgctggtgaggataacaacacgcctctcttcaacggcagacacccccatgctggcaggggtctccgagtatgaacttccagaggacccaaaatgggagtttccaagagataagctgacactgggcaagcccctgggagaaggttgctttgggcaagtggtcatggcggaagcagtgggaattgacaaagacaagcccaaggaggcggtcaccgtggccgtgaagatgttgaaagatgatgccacagagaaagacctttctgatctggtgtcagagatggagatgatgaagatgattgggaaacacaagaatatcataaatcttcttggagcctgcacacaggatgggcctctctatgtcatagttgagtatgcctctaaaggcaacctccgagaatacctccgagcccggaggccacccgggatggagtactcctatgacattaaccgtgttcctgaggagcagatgaccttcaaggacttggtgtcatgcacctaccagctggccagaggcatggagtacttggcttcccaaaaatgtattcatcgagatttagcagccagaaatgttttggtaacagaaaacaatgtgatgaaaatagcagactttggactcgccagagatatcaacaatatagactattacaaaaagaccaccaatgggcggcttccagtcaagtggatggctccagaagccctgtttgatagagtatacactcatcagagtgatgtctggtccttcggggtgttaatgtgggagatcttcactttagggggctcgccctacccagggattcccgtggaggaactttttaagctgctgaaggaaggacacagaatggataagccagccaactgcaccaacgaactgtacatgatgatgagggactgttggcatgcagtgccctcccagagaccaacgttcaagcagttggtagaagacttggatcgaattctcactctcacaaccaatgaggaatacttggacctcagccaacctctcgaacagtattcacctagttaccctgacacaagaagttcttgttcttcaggagatgattctgttttttctccagaccccatgccttacgaaccatgccttcctcagtatccacacataaacggcagtgttaaaacatga

SEQ ID NO: 22 - protein, 821 aa, Homo sapiens
MVSWGRFICLVVVTMATLSLARPSFSLVEDTTLEPEEPPTKYQISQPEVYVAAPGESLEVRCLLKDAAVISWTKDGVHLGPNNRTVLIGEYLQIKGATPRDSGLYACTASRTVDSETWYFMVNVTDAISSGDDEDDTDGAEDFVSENSNNKRAPYWTNTEKMEKRLHAVPAANTVKFRCPAGGNPMPTMRWLKNGKEFKQEHRIGGYKVRNQHWSLIMESVVPSDKGNYTCVVENEYGSINHTYHLDVVERSPHRPILQAGLPANASTVVGGDVEFVCKVYSDAQPHIQWIKHVEKNGSKYGPDGLPYLKVLKAAGVNTTDKEIEVLYIRNVTFEDAGEYTCLAGNSIGISFHSAWLTVLPAPGREKEITASPDYLEIAIYCIGVFLIACMVVTVILCRMKNTTKKPDFSSQPAVHKLTKRIPLRRQVTVSAESSSSMNSNTPLVRITTRLSSTADTPMLAGVSEYELPEDPKWEFPRDKLTLGKPLGEGCFGQVVMAEAVGIDKDKPKEAVTVAVKMLKDDATEKDLSDLVSEMEMMKMIGKHKNIINLLGACTQDGPLYVIVEYASKGNLREYLRARRPPGMEYSYDINRVPEEQMTFKDLVSCTYQLARGMEYLASQKCIHRDLAARNVLVTENNVMKIADFGLARDINNIDYYKKTTNGRLPVKWMAPEALFDRVYTHQSDVWSFGVLMWEIFTLGGSPYPGIPVEELFKLLKEGHRMDKPANCTNELYMMMRDCWHAVPSQRPTFKQLVEDLDRILTLTTNEEYLDLSQPLEQYSPSYPDTRSSCSSGDDSVFSPDPMPYEPCLPQYPHINGSVKT

SEQ ID NO: 23 - DNA, 2649 bp, Homo sapiens
atgggcccttggagccgcagcctctcggcgctgctgctgctgctgcaggtctcctcttggctctgccaggagccggagccctgccaccctggctttgacgccgagagctacacgttcacggtgccccggcgccacctggagagaggccgcgtcctgggcagagtgaattttgaagattgcaccggtcgacaaaggacagcctatttttccctcgacacccgattcaaagtgggcacagatggtgtgattacagtcaaaaggcctctacggtttcataacccacagatccatttcttggtctacgcctgggactccacctacagaaagttttccaccaaagtcacgctgaatacagtggggcaccaccaccgccccccgccccatcaggcctccgtttctggaatccaagcagaattgctcacatttcccaactcctctcctggcctcagaagacagaagagagactgggttattcctcccatcagctgcccagaaaatgaaaaaggcccatttcctaaaaacctggttcagatcaaatccaacaaagacaaagaaggcaaggttttctacagcatcactggccaaggagctgacacaccccctgttggtgtctttattattgaaagagaaacaggatggctgaaggtgacagagcctctggatagagaacgcattgccacatacactctcttctctcacgctgtgtcatccaacgggaatgcagttgaggatccaatggagattttgatcacggtaaccgatcagaatgacaacaagcccgaattcacccaggaggtctttaaggggtctgtcatggaaggtgctcttccaggaacctctgtgatggaggtcacagccacagacgcggacgatgatgtgaacacctacaatgccgccatcgcttacaccatcctcagccaagatcctgagctccctgacaaaaatatgttcaccattaacaggaacacaggagtcatcagtgtggtcaccactgggctggaccgagagagtttccctacgtataccctggtggttcaagctgctgaccttcaaggtgaggggttaagcacaacagcaacagctgtgatcacagtcactgacaccaacgataatcctccgatcttcaatcccaccacgtacaagggtcaggtgcctgagaacgaggctaacgtcgtaatcaccacactgaaagtgactgatgctgatgcccccaataccccagcgtgggaggctgtatacaccatattgaatgatgatggtggacaatttgtcgtcaccacaaatccagtgaacaacgatggcattttgaaaacagcaaagggcttggattttgaggccaagcagcagtacattctacacgtagcagtgacgaatgtggtaccttttgaggtctctctcaccacctccacagccaccgtcaccgtggatgtgctggatgtgaatgaagcccccatctttgtgcctcctgaaaagagagtggaagtgtccgaggactttggcgtgggccaggaaatcacatcctacactgcccaggagccagacacatttatggaacagaaaataacatatcggatttggagagacactgccaactggctggagattaatccggacactggtgccatttccactcgggctgagctggacagggaggattttgagcacgtgaagaacagcacgtacacagccctaatcatagctacagacaatggttctccagttgctactggaacagggacacttctgctgatcctgtctgatgtgaatgacaacgcccccataccagaacctcgaactatattcttctgtgagaggaatccaaagcctcaggtcataaacatcattgatgcagaccttcctcccaatacatctcccttcacagcagaactaacacacggggcgagtgccaactggaccattcagtacaacgacccaacccaagaatctatcattttgaagccaaagatggccttagaggtgggtgactacaaaatcaatctcaagctcatggataaccagaataaagaccaagtgaccaccttagaggtcagcgtgtgtgactgtgaaggggccgctggcgtctgtaggaaggcacagcctgtcgaagcaggattgcaaattcctgccattctggggattcttggaggaattcttgctttgctaattctgattctgctgctcttgctgtttcttcggaggagagcggtggtcaaagagcccttactgcccccagaggatgacacccgggacaacgtttattactatgatgaagaaggaggcggagaagaggaccaggactttgacttgagccagctgcacaggggcctggacgctcggcctgaagtgactcgtaacgacgttgcaccaaccctcatgagtgtcccccggtatcttccccgccctgccaatcccgatgaaattggaaattttattgatgaaaatctgaaagcggctgatactgaccccacagccccgccttatgattctctgctcgtgtttgactatgaaggaagcggttccgaagctgctagtctgagctccctgaactcctcagagtcagacaaagaccaggactatgactacttgaacgaatggggcaatcgcttcaagaagctggctgacatgtacggaggcggcgaggacgactag

SEQ ID NO: 24 - protein, 882 aa, Homo sapiens
MGPWSRSLSALLLLLQVSSWLCQEPEPCHPGFDAESYTFTVPRRHLERGRVLGRVNFEDCTGRQRTAYFSLDTRFKVGTDGVITVKRPLRFHNPQIHFLVYAWDSTYRKFSTKVTLNTVGHHHRPPPHQASVSGIQAELLTFPNSSPGLRRQKRDWVIPPISCPENEKGPFPKNLVQIKSNKDKEGKVFYSITGQGADTPPVGVFIIERETGWLKVTEPLDRERIATYTLFSHAVSSNGNAVEDPMEILITVTDQNDNKPEFTQEVFKGSVMEGALPGTSVMEVTATDADDDVNTYNAAIAYTILSQDPELPDKNMFTINRNTGVISVVTTGLDRESFPTYTLVVQAADLQGEGLSTTATAVITVTDTNDNPPIFNPTTYKGQVPENEANVVITTLKVTDADAPNTPAWEAVYTILNDDGGQFVVTTNPVNNDGILKTAKGLDFEAKQQYILHVAVTNVVPFEVSLTTSTATVTVDVLDVNEAPIFVPPEKRVEVSEDFGVGQEITSYTAQEPDTFMEQKITYRIWRDTANWLEINPDTGAISTRAELDREDFEHVKNSTYTALIIATDNGSPVATGTGTLLLILSDVNDNAPIPEPRTIFFCERNPKPQVINIIDADLPPNTSPFTAELTHGASANWTIQYNDPTQESIILKPKMALEVGDYKINLKLMDNQNKDQVTTLEVSVCDCEGAAGVCRKAQPVEAGLQIPAILGILGGILALLILILLLLLFLRRRAVVKEPLLPPEDDTRDNVYYYDEEGGGEEDQDFDLSQLHRGLDARPEVTRNDVAPTLMSVPRYLPRPANPDEIGNFIDENLKAADTDPTAPPYDSLLVFDYEGSGSEAASLSSLNSSESDKDQDYDYLNEWGNRFKKLADMYGGGEDD